(19) United States
(12) Patent Application Publication
(10) Pub. No.: US 2013/0245006 A1
(43) Pub. Date: Sep. 19, 2013

(54) NOVEL INDOLIZINE DERIVATIVES, AND PREPARATION AND THERAPEUTIC USE THEREOF

(71) Applicants: Jean-Michel Altenburger, Paris (FR); Franck Caussanel, Paris (FR); Sergio Mallart, Paris (FR); Marie-Claire Philippo-Orts, Paris (FR)

(72) Inventors: Jean-Michel Altenburger, Paris (FR); Franck Caussanel, Paris (FR); Sergio Mallart, Paris (FR); Marie-Claire Philippo-Orts, Paris (FR)

(73) Assignee: SANOFI, Paris (FR)

(21) Appl. No.: 13/885,866

(22) PCT Filed: Nov. 16, 2011
(86) PCT No.: PCT/FR2011/052661

§ 371 Date: May 16, 2013

(30) Foreign Application Priority Data
Nov. 17, 2010 (FR) 1059445

(51) Int. Cl. C07D 471/04

(52) U.S. Cl. CPC C07D 471/04
USPC 514/218; 546/112; 514/299; 544/362; 514/253.04; 540/492; 546/23

(57) ABSTRACT

The invention relates to indolizine derivatives of general formula (I), where A, B, m, W, n and R2 are as defined in claim 1, as well as to the method for preparing same and to the therapeutic use thereof.

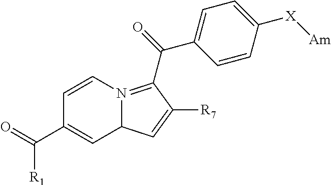

The subject of the present invention is novel indolizine derivatives, the process for preparing same and the therapeutic use thereof.

Atrial fibrillation (AF) is the most common arrhythmia and is associated with a high morbidity including heart failure and heart attacks. It is often encountered in patients exhibiting a cardiac pathological condition such as hypertension, or coronary artery disease or heart valve disease. The most significant consequences of AF are heart failure, with a 5-fold increase in the risk of heart attack and twice the risk of mortality (Duray G. Z., Ehrlich J. R., Hohnloser S. H., Dronedarone: a novel antiarrhythmic agent for the treatment of atrial fibrillation. Curr. Opin. Cardiol. 2010; 25: 53-58). Because of the aging of the population, the number of adults exhibiting AFs is likely to increase over the coming decades.

AF is characterized by the coexistence of numerous activation waves in the atrial myocardium. The mechanism of their initiation and of their persistence has been the subject of a great deal of discussion over the past few years. Because any form of tachyarrhythmia induces frequency-dependent remodeling, the coexistence of multiple reentry foci could represent the common mechanism responsible for the persistence of AFs associated with various pathological causes. According to the “leading circle” theory, the maintenance of reentries depends on the wavelength of the circuit which is the result of the conduction rate multiplied by the effective refractory period (ERP) within the circuit. The longer the wavelength, the fewer the possible number of AF circuits in the atrium and the higher the probability that the reentry circuits will be simultaneously interrupted. Thus, any medicament which prolongs the atrial. ERP should have antiarrhythmic properties (Ehrlich J. R., Nattel S, Novel approaches for pharmacological management of atrial fibrillation. Drugs 2009; 69: 757-774).

FR 2341578 and EP 471609 describe indolizine derivatives which have notable pharmacological properties, in particular antiarrhythmic properties, since these derivatives have proven to be capable of suppressing or preventing atrial rhythm disorders. Most compounds described have electrophysiological properties of classes 1, 2, 3 and 4 of the Vaughan-Williams classification, which confer, in addition to their antiarrhythmic properties, noncompetitive anti-α- and -β-adrenergic, anti-hypertensive and bradycardic properties. These properties make the compounds in question very useful in the treatment of certain pathological syndromes of the cardiovascular system, in particular in the treatment of angina pectoris, of hypertension, or of ventricular or supraventricular arrhythmia. Likewise, these compounds are used in the treatment of heart failure, or of myocardial infarction which may or may not be complicated by heart failure, or for the prevention of post-infarction mortality.

Nevertheless, these compounds have the drawback being insoluble or sparingly soluble in water.

Amiodarone, which is an auricular and ventricular antiarrhythmic that is active orally and intravenously, is a water-insoluble molecule; the injectable solution therefore contains solvents such as polysorbate 80 and benzyl alcohol. These solvents induce hypotensive and negative inotropic effects in the patient. The injectable solution also causes local venous intolerance, which is avoided by recommending a central injection in a specialized hospital environment.

Dronedarone, a benzofuran derivative, which does not contain iodine in its chemical structure unlike amiodarone, is also an auricular and ventricular antiarrhythmic which is active orally and intravenously.

In the context of the invention, antiarrhythmics which are active orally, of indolizine type, capable of blocking several ion channels like dronedarone but without its limitations and drawbacks, have now been discovered. The biggest disadvantage of dronedarone is its contraindication in patients with heart failure. It is probable that these effects are linked to the blockage of sodium channels (Lalevee N., Nargeot J., Barrere-Lemaire S., Gautier P., Richard S. Effects of amiodarone and Dronedarone on voltage-dependent sodium current in human cardiomyocytes. J. Cardiovasc. Electrophysiol. 2003; 14:885-890) and calcium channels (Gautier P., Guillemare E., Marion A., Bertrand J. P., Tourneur Y., Nisato D. Electrophysiologic characterization of Dronedarone in guinea pig ventricular cells. J. Cardiovasc. Pharmacol. 2003; 41: 191-202) causing negative inotropy in animals and probably also in patients. Consequently, the new compounds will have to be free of any negative inotropy effect in animals (pigs, for example). Furthermore, compared with amiodarone or with dronedarone, our compounds offer better metabolic stability and a stability in water that is sufficient for an injectable form.

A subject of the present invention is compounds corresponding to formula (I):

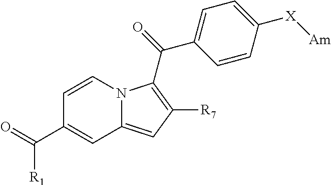

wherein
R1 represents:

either

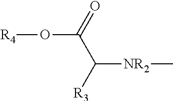

or

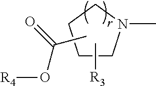

or

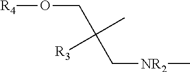

or

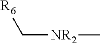

or

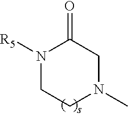

or

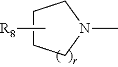

or

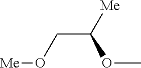

or

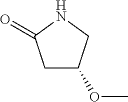

or

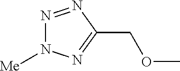

R2 represents a hydrogen atom, a (C₁-C₆) alkyl group, a benzyl group or a CH₂—CF₃ group;
R3 represents a hydrogen atom, a (C₁-C₆) alkyl group or a benzyl group;
R4 represents a hydrogen atom or a (C₁-C₄) alkyl group;
R5 represents a hydrogen atom or a (C₁-C₅) alkyl group;
R6 represents a nitrile group or a heteroaryl group comprising from 1 to 4 heteroatoms chosen from a nitrogen atom and an oxygen atom, this heteroaryl group being optionally substituted with a (C₁-C₆) alkyl group;
R7 represents a hydrogen atom or a linear, branched or cyclic (C₁-C₆) alkyl group;
R8 represents a hydroxyl group or a cyano group;
X represents a bond or an oxygen atom;
Am represents:

either

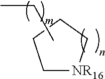

or

—(CH₂)ₜ—CR₁₉R₂₀NR₁₇—R₁₈

R₁₆ represents a hydrogen atom or a (C₁-C₆) alkyl group;
R17 represents a hydrogen atom or a (C₁-C₆) alkyl group;
R18 represents a branched or cyclic (C₁-C₆) alkyl group;
R19 and R20 represent a hydrogen atom or a (C₁-C₆) alkyl group, or form a (C₃-C₆) spiroalkyl group;
m represents an integer equal to 0 or 1;
n represents an integer equal to 1 or 2;
r represents an integer equal to 1 or 2;
s represents an integer equal to 1 or 2;
t represents an integer between 2 and 4.

The compounds of formula (I) can comprise one or more asymmetric carbon atoms. They can therefore exist in the form of enantiomers or of diastereoisomers. These enantiomers and diastereoisomers, and also mixtures thereof, including racemic mixtures, form part of the invention.

The compounds of formula (I) can exist in the form of bases or in a form salified with acids or bases, in particular pharmaceutically acceptable acids or bases. Such addition salts form part of the invention.

These salts are advantageously prepared with pharmaceutically acceptable acids, but the salts of other acids useful, for example, for purifying or isolating the compounds of formula (I) also form part of the invention.

In the context of the present invention, and unless otherwise mentioned in the text:
the term “a halogen atom” is intended to mean: a fluorine, a chlorine, a bromine or an iodine;
the term “an alkyl group” is intended to mean: a linear, branched or cyclic saturated aliphatic group. By way of examples, mention may be made of methyl, ethyl, propyl, isopropyl, butyl, isobutyl, tert-butyl, pentyl, etc. groups;

the term “a spiroalkyl group” is intended to mean: a bicycle of which the rings are connected via a single atom. The rings may be of identical or different length or nature;
the term “a haloalkyl group” is intended to mean: an alkyl group of which one or more hydrogen atoms has (have) been substituted with a halogen atom;
the term “an aryl group” is intended to mean: a cyclic aromatic group comprising between 6 and 10 carbon atoms. By way of examples of aryl groups, mention may be made of a phenyl, benzyl or naphthyl;
the term “a heteroaryl group” is intended to mean: a cyclic aromatic group comprising 2, 3, 4 or 5 carbon atoms and comprising from 1 to 4 heteroatoms chosen from a nitrogen atom and an oxygen atom, independently of one another, so as to be identical or different, when there are 2 of them, or independently of one another, so as to be identical or different, when there are 3 of them. Mention may be made of pyridyl, furanyl and pyrrolyle groups;
the term “a hydroxyl group” is intended to mean: an —OH group.

Among the compounds of formula (I) which are subjects of the invention, mention may in particular be made of the following compounds:
compound No. 2: (S)-1-{2-Butyl-3-[4-(3-dibutylamino-propyl)benzoyl]indolizine-7-carbonyl}-pyrrolidine-2-carboxylic acid methyl ester;
compound No. 3: (R)-1-{2-Butyl-3-[4-(3-dibutylamino-propyl)benzoyl]indolizine-7-carbonyl}pyrrolidine-2-carboxylic acid methyl ester;
compound No. 4: 2-Butyl-3-[4-(3-dibutylaminopropyl)benzoyl]indolizine-7-carboxylic acid ethyl (2-methoxyethyl)amide;
compound No. 5: ({2-Butyl-3-[4-(3-dibutylaminopropyl)benzoyl]indolizine-7-carbonyl}ethylamino)acetic acid methyl ester;
compound No. 6: ({2-Butyl-3-[4-(3-dibutylaminopropyl)benzoyl]indolizine-7-carbonyl}ethylamino)acetic acid;
compound No. 7: 3-({2-Butyl-3-[4-(3-dibutylaminopropyl)benzoyl]indolizine-7-carbonyl}ethylamino)propionic acid;
compound No. 8: ({3-[4-(3-Cyclopentylaminopropyl)benzoyl]-2-ethylindolizine-7-carbonyl}isopropylamino)acetic acid methyl ester;
compound No. 9: 2-Butyl-3-[4-(3-dibutylaminopropyl)benzoyl]indolizine-7-carboxylic acid ethyl(2-methyl-2H-tetrazol-5-ylmethyl)amide;
compound No. 10: 2-Butyl-3-[4-(3-dibutylaminopropyl)benzoyl]indolizine-7-carboxylic acid ethyl(3-methyl-[1,2,4]oxadiazol-5-ylmethyl)amide;
compound No. 11: 2-Butyl-3-[4-(3-dibutylaminopropyl)benzoyl]indolizine-7-carboxylic acid ethyl(1H-tetrazole-5-ylmethyl)amide;
compound No. 12: {[2-Butyl-3-(4-piperidin-4-ylbenzoyl)indolizine-7-carbonyl]isopropylamino}acetic acid methyl ester;
compound No. 13: 4-{2-Butyl-3-[4-(3-dibutylamino-propyl)benzoyl]indolizine-7-carbonyl}piperazine-2-one;
compound No. 14: ({3-[4-(4-Cyclopentylaminobutyl)benzoyl]-2-ethylindolizine-7-carbonyl}isopropylamino)acetic acid methyl ester;
compound No. 15: [(3-{4-[3-(1-Aminocyclopentyl)propyl]benzoyl}-2-ethylindolizine-7-carbonyl)isopropylamino]acetic acid methyl ester;
compound No. 16: [(2-Ethyl-3-(4-[3-(1-methylaminocyclopentyl)propyl]benzoyl}indolizine-7-carbonyl)isopropylamino]acetic acid methyl ester;
compound No. 17: 3-[4-(3-tert-Butylaminopropyl)benzoyl]-2-ethylindolizine-7-carboxylic acid ethyl(2-methyl-2H-tetrazol-5-ylmethyl)amide;
compound No. 18: 3-[4-(3-tert-Butylamino-3-methylbutyl)benzoyl]-2-ethylindolizine-7-carboxylic acid ethyl(2-methyl-2H-tetrazol-5-ylmethyl)amide;
compound No. 19: ({3-[4-(3-Cyclopentylamino-3-methylbutyl)benzoyl]-2-ethylindolizine-7-carbonyl}isopropylamino)acetic acid methyl ester;
compound No. 20: 2-Ethyl-3-[4-((S)-3-ethylamino-4-methylpentyl)benzoyl]indolizine-7-carboxylic acid ethyl(2-methyl-2H-tetrazol-5-ylmethyl)amide;
compound No. 21: 2-Butyl-3-[4-(3-dibutylaminopropyl)benzoyl]indolizine-7-carboxylic acid benzyl(2-methoxyethyl)amide;
compound No. 22: 2-Butyl-3-[4-(3-dibutylaminopropyl)benzoyl]indolizine-7-carboxylic acid isopropyl(2-methoxyethyl)amide;
compound No. 23: 2-Butyl-3-[4-(3-dibutylaminopropyl)benzoyl]indolizine-7-carboxylic acid ethyl(2-isopropoxyethyl)amide;
compound No. 24: 2-Butyl-3-[4-(3-dibutylaminopropyl)benzoyl]indolizine-7-carboxylic acid (2-ethoxyethyl)isopropylamide;
compound No. 25: 2-Butyl-3-[4-(3-dibutylaminopropyl)benzoyl]indolizine-7-carboxylic acid (2-methoxyethyl)(2,2,2-trifluoroethyl)amide;
compound No 26: ({2-Butyl-3-[4-(3-dibutylamino-propyl)benzoyl]indolizine-7-carbonyl}ethylamino)acetic acid ethyl ester;
compound No. 27: ({2-Butyl-3-[4-(3-dibutylamino-propyl)benzoyl]indolizine-7-carbonyl}ethylamino)acetic acid isopropyl ester;
compound No. 28: ({2-Butyl-3-[4-(3-dibutylamino-propyl)benzoyl]indolizine-7-carbonyl}isopropyl-amino)acetic acid methyl ester;
compound No. 29: ({3-[4-(3-dibutylamino-propyl)benzoyl]-2-ethylindolizine-7-carbonyl}isopropylamino)acetic acid methyl ester;
compound No. 30: ({3-[4-(3-Butylaminopropyl)benzoyl]-2-ethylindolizine-7-carbonyl}isopropylamino) acetic acid methyl ester;
compound No. 31: ({3-[4-(3-tert-Butylaminopropyl)benzoyl]-2-ethylindolizine-7-carbonyl}isopropylamino)acetic acid methyl ester;
compound No. 32: ({2-Ethyl-3-[4-(3-isopropylaminopropyl)benzoyl]indolizine-7-carbonyl}isopropylamino)acetic acid methyl ester;
compound No. 33: ({3-[4-(3-Cyclopentylaminopropyl)benzoyl]-2-ethylindolizine-7-carbonyl}ethylamino)acetic acid ethyl ester;
compound No. 34: ({3-[4-(3-Cyclopentylamino-propyl)benzoyl]-2-isopropylindolizine-7-carbonyl}isopropylamino)acetic acid methyl ester;
compound No. 35: ({3-[4-(3-Cyclohexylaminopropyl)benzoyl]-2-ethylindolizine-7-carbonyl}isopropylamino)acetic acid methyl ester;
compound No. 36: [(3-{4-[3(2,2-Dimethylpropyl-amino)propyl]benzoyl}-2-ethylindolizine-7-carbonyl)isopropylamino]acetic acid methyl ester;
compound No. 37: 3-[4-(3-Cyclopentylaminopropyl)benzoyl]-2-ethylindolizine-7-carboxylic acid (2-ethoxyethyl)ethylamide;
compound No. 38: 4-{3-[4-(3-tert-Butylaminopropyl)benzoyl]-2-ethylindolizine-7-carbonyl}piperazin-2-one;
compound No. 39: 2-Ethyl-3-{4-[3-(1-methylcyclopentylamino)propyl]benzoyl}indolizine-7-carboxylic acid ethyl(2-methyl-2H-tetrazol-5-yl-methyl)amide;
compound No. 40: 3-[4-(3-tert-Butylaminopropyl)benzoyl]-2-ethylindolizine-7-carboxylic acid ethyl(2-ethyl-2H-tetrazol-5-yl-methyl)amide;
compound No. 41: 3-[4-(3-tert-Butylaminopropyl)benzoyl]indolizine-7-carboxylic acid ethyl(2-methyl-2H-tetrazol-5-ylmethyl)amide;
compound No. 42: 3-[4-(3-tert-Butylaminopropyl)benzoyl]-2-cyclobutylindolizine-7-carboxylic acid ethyl(2-methyl-2H-tetrazol-5-ylmethyl)amide;
compound No. 43: 2-Ethyl-3-[4-(3-ethylamino-4,4-dimethylpentyl)benzoyl]indolizine-7-carboxylic acid ethyl(2-methyl-2H-tetrazol-5-ylmethyl)amide;
compound No. 44: 3-[4-(3-tert-Butylaminopropyl)benzoyl]-2-ethylindolizine-7-carboxylic acid ethyl(1-methyl-1H-pyrazol-3-ylmethyl)amide;
compound No. 45: 3-[4-(3-tert-Butylaminopropyl)benzoyl]-2-ethylindolizine-7-carboxylic acid ethyl(1-methyl-1H-pyrazol-4-ylmethyl)amide;
compound No. 46: 3-[4-(3-tert-Butylaminopropyl)benzoyl]-2-ethylindolizine-7-carboxylic acid ethyl(5-methylisoxazol-3-ylmethyl)amide;
compound No. 47: (R)-1-{3-[4-(3-tert-Butylamino-propyl)benzoyl]-2-ethylindolizine-7-carbonyl}pyrrolidine-3-carbonitrile;
compound No. 48: {3-[4-(3-tert-Butylamino-propyl)benzoyl-2-ethylindolizin-7-yl}-((S)-3-hydroxypyrrolidin-1-yl)methanone;
compound No. 49: 3-[4-(3-tert-Butylaminopropyl)benzoyl]-2-ethylindolizine-7-carboxylic acid 2-methyl-2H-tetrazol-5-ylmethyl ester;
compound No. 50: (S)-1-{3-[4-(3-tert-Butylaminopropyl)benzoyl]-2-ethylindolizine-7-carbonyl}-2-methylpyrrolidine-2-carboxylic acid methyl ester;
compound No. 51: 3-[4-(3-tert-Butylamino-propyl)benzoyl]-2-ethylindolizine-7-carboxylic acid (R)-2-methoxy-1-methylethyl ester;
compound No. 52: 3-[4-(3-tert-Butylaminopropyl)benzoyl]-2-ethylindolizine-7-carboxylic acid (R)-5-oxopyrrolidin-3-yl ester;
compound No. 53: 1-{3-[4-(3-Cyclopentylaminopropyl)benzoyl]-2-ethylindolizine-7-carbonyl}[1,4]diazepam-5-one;
compound No. 54: [(3-{4-[3-(tert-Butylmethylamino)propyl]benzoyl}-2-ethylindolizine-7-carbonyl)isopropylamino]acetic acid methyl ester;
compound No. 55: [(2-Ethyl-3-{4-[3-(ethylisopropylamino)propyl]benzoyl}indolizine-7-carbonyl)isopropylamino]acetic acid methyl ester;
compound No. 56: ({3-[4-(3-Dibutylamino-propyl)benzoyl]indolizine-7-carbonyl}isopropyl-amino)acetic acid methyl ester;
compound No. 57: ({3-[4-(3-Dibutylaminopropyl)benzoyl]indolizine-7-carbonyl}isopropylamino)acetic acid;
compound No. 58: ({2-Butyl-3-[4-(3-dibutylaminopropyl)benzoyl]indolizine-7-carbonyl}isopropylamino)acetic acid isopropyl ester;
compound No. 59: 2-Butyl-3-(4-piperidin-4-ylbenzoyl)indolizine-7-carboxylic acid diethylamide;
compound No. 60: ({2-Butyl-3-[4-(3-dibutylamino-propyl)benzoyl]indolizine-7-carbonyl}isopropyl amino) acetic acid ethyl ester;
compound No. 61: ({3-[4-(3-Dipropylamino-propyl)benzoyl]-2-ethylindolizine-7-carbonyl}isopropylamino)acetic acid methyl ester;
compound No. 62: [(2-Butyl-3-{4-[3-(butylethylamino)propyl]benzoyl}indolizine-7-carbonyl)isopropylamino]acetic acid methyl ester;
compound No. 63: ({2-Butyl-3-[4-(3-dibutylamino-propyl)benzoyl]indolizine-7-carbonyl}isobutyl-amino)acetic acid methyl ester;
compound No. 64: ({2-Butyl-3-[4-(1-methylpiperidin-4-yl)benzoyl]indolizine-7-carbonyl}isopropylamino)acetic acid;
compound No. 65: {[2-Ethyl-3-(4-piperidin-4-yl-benzoyl)indolizine-7-carbonyl]isopropylamino}acetic acid methyl ester;
compound No. 66: 2-Butyl-3-(4-piperidin-4-yl-benzoyl) indolizine-7-carboxylic acid diethylamide;
compound No. 67: ({2-Butyl-3-[4-(piperidin-4-yloxy)benzoyl]indolizine-7-carbonyl}isopropyl-amino)acetic acid methyl ester;
compound No. 68: ({2-Butyl-3-[4-((S)-piperidin-3-yloxy)benzoyl]indolizine-7-carbonyl}isopropyl-amino)acetic acid methyl ester;
compound No. 69: (S)-2-({2-Butyl-3-[4-(3-dibutylaminopropyl)benzoyl]indolizine-7-carbonyl}ethyl-amino)propionic acid methyl ester;
compound No. 70: 2-Butyl-3-[4-(3-dibutylaminopropyl)benzoyl]indolizine-7-carboxylic acid benzylethylamide;
compound No. 71: 2-Butyl-3-[4-(3-butylamino-propyl)benzoyl]indolizine-7-carboxylic acid ethyl(2-methoxyethyl)amide;
compound No. 72: 2-Butyl-3-[4-(3-dibutylamino-propyl)benzoyl]indolizine-7-carboxylic acid (2-isopropoxyethyl)isopropylamide;
compound No. 73: [(2-Butyl-3-{4-[3-((3R,5S)-3,5-dimethylpiperidin-1-yl)propyl]benzoyl}indolizine-7-carbonyl)isopropylamino]acetic acid methyl ester;
compound No. 74: (Benzyl-{2-butyl-3-[4-(3-dibutyl-aminopropyl)benzoyl]indolizine-7-carbonyl}amino)acetic acid methyl ester;
compound No. 75: ({2-Butyl-3-[4-(3-diethylamino-propyl)benzoyl]indolizine-7-carbonyl}isopropylamino)acetic acid;
compound No. 76: ({3-[4-(3-tert-Butylamino-propyl)benzoyl]-2-ethylindolizine-7-carbonyl}isopropylamino)acetic acid;
compound No. 77: 3-[4-(3-Cyclopentyl-aminopropyl)benzoyl]-2-ethylindolizine-7-carboxylic acid ethyl(2-methyl-2H-tetrazol-5-ylmethyl)amide;
compound No. 78: 3-[4-(3-tert-Butylaminopropyl)benzoyl]-2-methylindolizine-7-carboxylic acid ethyl(2-methyl-2H-tetrazol-5-ylmethyl)amide;
compound No. 79: [(2-Ethyl-3-{4-[3-(1-isopropylamino-cyclopentyl)propyl]benzoyl}indolizine-7-carbonyl)isopropylamino]acetic acid methyl ester;
compound No. 80: 2-Butyl-3-(4-piperidin-4-ylbenzoyl)indolizine-7-carboxylic acid ethyl(3-methyl-[1,2,4]oxadizol-5-ylmethyl)amide;
compound No. 81: ({2-Butyl-3-[4-((R)-piperidin-3-yloxy)benzoyl]indolizine-7-carbonyl}isopropyl-amino)acetic acid methyl ester;
   in the form of a base or of an addition salt with an acid.

Among the compounds of formula (I) which are subjects of the invention, one group of compounds consists of the following compounds:
compound No. 3: (R)-1-{2-Butyl-3-[4-(3-dibutylamino-propyl)benzoyl]indolizine-7-carbonyl}pyrrolidine-2-carboxylic acid methyl ester;
compound No. 4: 2-Butyl-3-[4-(3-dibutylamino-propyl)benzoyl]indolizine-7-carboxylic acid ethyl(2-methoxyethyl)amide;
compound No. 5: ({2-Butyl-3-[4-(3-dibutylamino-propyl)benzoyl]indolizine-7-carbonyl}ethylamino)acetic acid methyl ester;
compound No. 8: ({3-[4-(3-Cyclopentylamino-propyl)benzoyl]2-ethylindolizine-7-carbonyl}isopropylamino)acetic acid methyl ester;
compound No. 9: 2-Butyl-3-[4-(3-dibutylamino-propyl)benzoyl]indolizine-7-carboxylic acid ethyl(2-methyl-2H-tetrazol-5-ylmethyl)amide;
compound No. 10: 2-Butyl-3-[4-(3-dibutylaminopropyl)benzoyl]indolizine-7-carboxylic acid ethyl(3-methyl[1,2,4]oxadizol-5-ylmethyl)amide;
compound No. 13: 4-{2-Butyl-3-[4-(3-dibutylamino-propyl)benzoyl]indolizine-7-carbonyl}piperazin-2-one;
compound No. 16: [(2-Ethyl-3-{4-[3-(1-methylaminocyclopentyl)propyl]benzoyl}indolizine-7-carbonyl)isopropylamino]acetic acid methyl ester;
compound No. 17: 3-[4-(3-tert-Butylamino-propyl)benzoyl]-2-ethylindolizine-7-carboxylic acid ethyl(2-methyl-2H-tetrazol-5-ylmethyl)amide;
compound No. 18: 3-[4-(3-tert-Butylamino-3-methyl-butyl)benzoyl]-2-ethylindolizine-7-carboxylic acid ethyl(2-methyl-2H-tetrazol-5-ylmethyl)amide;
compound No. 22: 2-Butyl-3-[4-(3-dibutylamino-propyl)benzoyl]indolizine-7-carboxylic acid isopropyl(2-methoxyethyl)amide;
compound No. 23: 2-Butyl-3-[4-(3-dibutylamino-propyl)benzoyl]indolizine-7-carboxylic acid ethyl(2-isopropoxyethyl)amide;
compound No. 24: 2-Butyl-3-[4-(3-dibutylaminopropyl)benzoyl]indolizine-7-carboxylic acid (2-ethoxy-ethyl)isopropylamide;
compound No. 28: ({2-Butyl-3-[4-(3-dibutylamino-propyl)benzoyl]indolizine-7-carbonyl}isopropyl-amino)acetic acid methyl ester;
compound No. 29: ({3-[4-(3-Dibutylamino-propyl)benzoyl]-2-ethylindolizine-7-carbonyl}iso-propylamino)acetic acid methyl ester;
compound No. 30: ({3-[4-(3-Butylaminopropyl)benzoyl]-2-ethylindolizine-7-carbonyl}isopropylamino)acetic acid methyl ester;
compound No. 31: ({3-[4-(3-tert-Butylamino-propyl)benzoyl]-2-ethylindolizine-7-carbonyl}iso-propylamino)acetic acid methyl ester;
compound No. 35: ({3-[4-(3-Cyclohexylamino-propyl)benzoyl]-2-ethylindolizine-7-carbonyl}isopropylamino)acetic acid methyl ester;
compound No. 40: 3-[4-(3-tert-Butylaminopropyl)benzoyl]-2-ethylindolizine-7-carboxylic acid ethyl(2-ethyl-2H-tetrazol-5-ylmethyl)amide;
compound No. 42: 3-[4-(3-tert-Butylaminopropyl)benzoyl]-2-cyclobutylindolizine-7-carboxylic acid ethyl(2-methyl-2H-tetrazol-5-ylmethyl)amide;
compound No. 43: 2-Ethyl-3-[4-(3-ethylamino-4,4-di-methylpentyl)benzoyl]indolizine-7-carboxylic acid ethyl(2-methyl-2H-tetrazol-5-ylmethyl)amide;
compound No. 53: 1-{3-[4-(3-Cyclopentylaminopropyl)benzoyl]-2-ethylindolizine-7-carbonyl}[1,4]diazepam-5-one;
compound No. 55: [(2-Ethyl-3-{4-[3-(ethylisopropyl-amino)propyl]benzoyl}indolizine-7-carbonyl) isopropyl-amino]acetic acid methyl ester;
compound No. 58: ({3-[4-(3-Dibutylamino-propyl)benzoyl]-2-ethylindolizine-7-carbonyl}ethyl-amino)acetic acid isopropyl ester;
compound No. 62: [(2-Butyl-3-{4-[3-(butylethylamino)propyl]benzoyl}indolizine-7-carbonyl)isopropylamino]acetic acid methyl ester;
compound No. 63: ({2-Butyl-3-[4-(3-dibutylamino-propyl)benzoyl]indolizine-7-carbonyl}isobutyl-amino)acetic acid methyl ester;
compound No. 64: ({2-Butyl-3-[4-(1-methylpiperidin-4-yl)benzoyl]indolizine-7-carbonyl}isopropylamino)acetic acid
compound No. 65: {[2-Ethyl-3-(4-piperidin-4-ylbenzoyl)indolizine-7-carbonyl]isopropylamino}acetic acid methyl ester;
compound No. 69: (S)-2-({2-Butyl-3-[4-(3-dibutyl-aminopropyl)benzoyl]indolizine-7-carbonyl}ethyl-amino)propionic acid methyl ester;
compound No. 75: ([2-Butyl-3-[4-(3-diethylamino-propyl)benzoyl]indolizine-7-carbonyl]isopropyl-amino)acetic acid;
compound No. 77: 3-[4-(3-Cyclopentylaminopropyl)benzoyl]-2-ethylindolizine-7-carboxylic acid ethyl(2-methyl-2H-tetrazol-5-ylmethyl)amide;
compound No. 78: 3-[4-(3-tert-Butylamino-propyl)benzoyl]-2-methylindolizine-7-carboxylic acid ethyl(2-methyl-2H-tetrazol-5-ylmethyl)amide; in the form of a base or of an addition salt with an acid.

In the subsequent text, the term “protective group” (Pg) is intended to mean a group which makes it possible, on the one hand, to protect a reactive function, such as a hydroxyl or an amine, during a synthesis and, on the other hand, to regenerate the intact reactive function at the end of synthesis. Examples of protective groups and also of methods of protection and deprotection are given in “Protective Groups in Organic Synthesis”, Green et al., 3^{rd} edition (John Wiley & Sons, Inc., New York).

In the text which follows, the term “Leaving group” (Lg) is intended to mean a group which can be readily cleaved from a molecule by breaking a heterolytic bond, with the departure of a pair of electrons. This group can thus be readily replaced with another group in a substitution reaction, for example. Such leaving groups are, for example, halogens or an activated hydroxyl group such as a mesyl, tosyl, triflate, acetyl, etc. Examples of leaving groups and also references for the preparation thereof are given in “Advances in Organic Chemistry”, J. March, 3^{rd} edition, Wiley Interscience, p. 310-316.

In accordance with the invention, it is possible to prepare the compounds of formula (I) with R₁, R₇, X and Am having the same meaning as previously according to the process which follows, illustrated in schemes 1, 2, 3, 4 and 5.

In schemes 1, 2, 3, 4 and 5, the starting compounds and the reagents, when the method for preparing same is not described, are commercially available or described in the literature, or else can be prepared according to methods which are described therein and which are known to those skilled in the art.

The indolizine nucleus (VIII, scheme 1) is prepared according to the Chichibabin process via the quaternization of the pyridine (XI) (with R=an alkyl group such as isopropyl) with an α-haloketone derivative (X) such as 1-bromohexan-2-one (Y=Br, step ii), in a solvent such as butan-2-one brought to reflux, followed by a cyclization reaction (step iii) in the presence of a base such as sodium carbonate, in a protic solvent such as isopropanol brought to reflux.

A Friedel-Crafts reaction for acylation of position 3 with an acid chloride (VII) where Y represents a halogen atom (step iv) gives, after heating, the ketone derivative (VI). Alternatively, in step (iv′), the acylation can be carried out with an acid chloride (VII′) with X-Am bearing an amine function which is masked in an amide or formate group, and which is unmasked at the end of synthesis so as to give the compound (I), or again used in a second protective group compatible with the saponification conditions of step (vi) and freed again after the final step (vii).

In a step (v), the condensation of the amine Am-H (V) with the halogenated derivative (VI), where Y represents a halogen atom, in the presence of a base such as potassium carbonate and of a catalytic amount of potassium iodide (KI), in a solvent such as acetonitrile brought to reflux, gives the amine (IV) which corresponds to a compound of the formula (I) wherein R₁ represents OR when R═R₁₂=a (C₁-C₆) alkyl group such as an isopropyl group.

The saponification (step vi) of the ester (IV) with sodium hydroxide in a solvent such as dioxane, followed by the activation (step vii) of the corresponding carboxylic acid (III) (which corresponds to a compound of formula (I) wherein R₁ represents O—R₁₂ with R₁₂=H) with a coupling agent such as O-benzotriazolyl-N,N,N′,N′-tetramethyluronium tetrafluoroborate (TBTU), in the presence of the amine derivative or alcohol derivative R₁—H (II) and of a base such as N,N-diisopropylethylamine (DIEA), in an aprotic solvent such as dichloromethane (DCM), gives the compounds of general formula (I) in accordance with the present invention.

Alternatively, as described in scheme 2, the X-Am chain can be introduced using a Sonogashira reaction between an alkyne derivative (V) and a halogenated derivative (VI), prepared as described in scheme 1, where Z represents a halogen, preferably an iodine atom. Thus, in step (ii), the coupling is carried out in the presence of an organic base such as DIEA and of a catalytic amount of copper (I) such as CuI or CuBr, and of palladium such as PdCl₂(PPh₃) in a polar solvent such as acetonitrile heated to 50° C. In step (iii), the triple bond of the alkyne derivative (IV) is then totally reduced under a hydrogen atmosphere or with a hydrogen-transferring agent such as ammonium formate, in the presence of a catalytic amount of palladium-on-carbon (Pd—C) in a protic solvent such as ethanol or methanol. Finally, as described in scheme 1, the ester (III) is subjected to a saponification-peptide coupling sequence (steps iv and v) to give the compound I. The various substituents, when their definition is not specified, are as defined in general formula (I).

Alternatively, as described in scheme 3, the Sonogashira coupling can be carried out with an alkyne derivative functionalized with a precursor of amine function such as a carboxylic acid function which is masked in a hydrogenolyzable benzyl ester group so as to guarantee effective orthogonal deprotection in the presence of the second ester function with R as previously described. Thus, in step (ii), the triple bond and the benzyl ester (R13=OBn) of the Sonogashira product are concomitantly reduced under a hydrogen atmosphere or with a hydrogen-transferring agent, such as ammonium formate, in the presence of a catalytic amount of palladium-on-carbon (Pd—C) in a protic solvent such as ethanol or methanol. The carboxylic acid function (VII) thus freed is converted, in step (iii) according to a Curtius rearrangement, into a tert-butyl carbamate function (V) in the presence of diphenylphosphoryl azide (DPPA) in tert-butyl alcohol and of a catalytic amount of copper (I) such as CuCl. The derivative (V) in steps (iv) and (v) is subjected, as described in scheme 1, to a saponification-peptide coupling sequence so as to give the compound (III).

Finally, the tert-butyl carbamate function (III) can be either acidolyzed with trifluoroacetic acid or hydrogen chloride in step (vii) so as to result in the compound (I) with R17=H, or, in step (vi), before the acidolysis step, treated with an inorganic base such as sodium hydride (NaH) in the presence of an alkyl halide R17X, with X representing a bromium or iodine atom, in a polar aprotic solvent such as dimethylformamide (DMF) and give the alkylated carbamate derivative (II).

The various substituents, when their definition is not specified, are as defined in general formula (I).

Alternatively, as described in scheme 4, the X-Am chain, with X representing an oxygen atom and Am functionalized with a protected amine function such as a tert-butyl carbamate, which guarantees good stability during the step of saponification of the ester function with R as described previously, is introduced using a Buchwald reaction so as to create a carbon-oxygen bond. Thus, in step (i), the coupling between the derivative (VI), with Z representing a halogen atom such as an iodine atom and HXAm (V) is carried out in the presence of an inorganic base such as cesium carbonate (Cs₂CO₃) and of a catalytic amount of a ligand of phenanthrolidine type and of copper (I), such as CuI, in an apolar solvent such as toluene heated at reflux. The ether (IV) in step (iii) is converted into the derivative (II) according to a saponification-peptide coupling sequence as described in scheme 1. Finally, the tert-butyl carbamate group is acidolyzed either with trifluoroacetic acid or with hydrogen chloride in an aprotic solvent such as methylene chloride (CH₂Cl₂) or ethyl acetate (EtOAC) so as to give the derivative I.

Alternatively, as described in scheme 5, the XAm chain can be introduced using a Wittig reaction. In step (i), the acylation reaction between the indolizine (VIII) and the acid chloride (IX), with X representing a halogen atom such as a chlorine atom, in the presence of an organic base such as lutidine and of pyridine in catalytic amounts in an aprotic solvent such as chlorobenzene heated at reflux, gives the compound (VII). In step (ii), according to the Arbuzov conditions, the benzyl halide derivative (VII) treated in an excess of phosphite derivative (V) such as ethyl phosphite heated at reflux, is converted into the phosphonate (VI). A Wittig reaction in step (iii) between the ester (VI) and a chiral α-aminoaldehyde derivative (IV), prepared from the α-amino acid parent compound of which the amine function is protected with a tert-butyl carbamate group for a final deprotection in an aprotic acidic medium, and from an inorganic base such as NaH, in an aprotic solvent such as THF, gives the alkene (III). In step (v), the alkene is converted, according to a hydrogenation-saponification-peptide coupling-acidolysis sequence, as described in scheme 2, into the derivative (I). The various substituents, when their definition is not specified, are as defined in general formula (I).

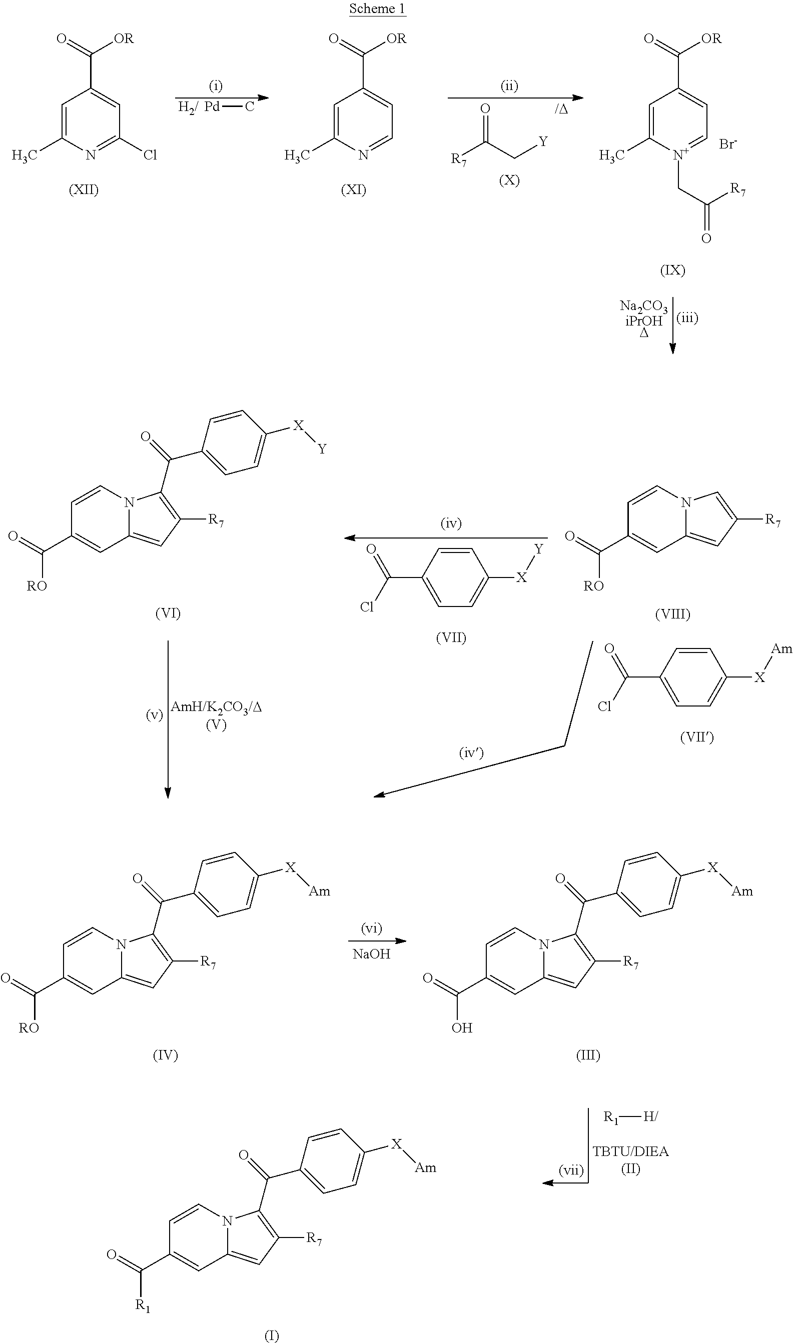

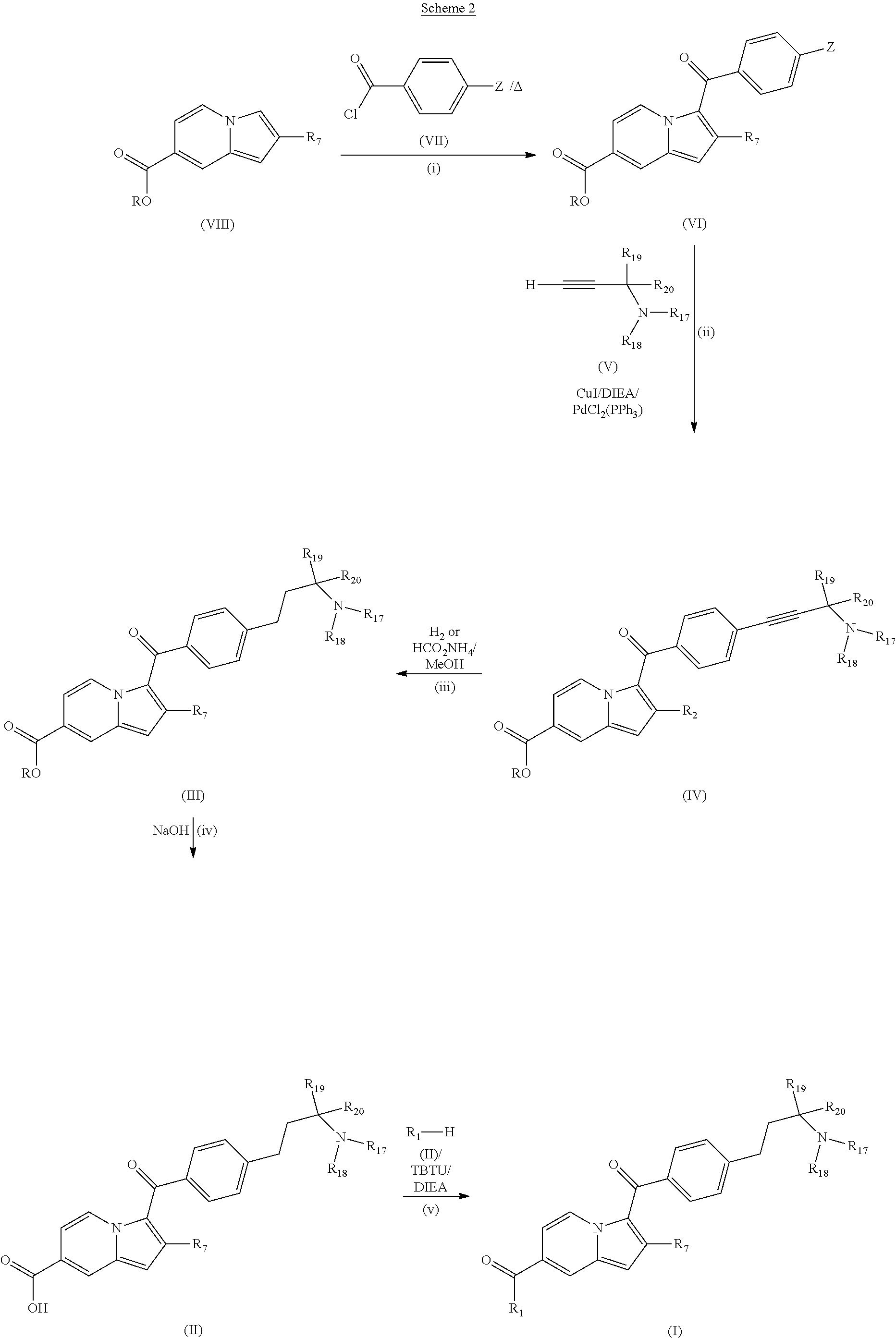

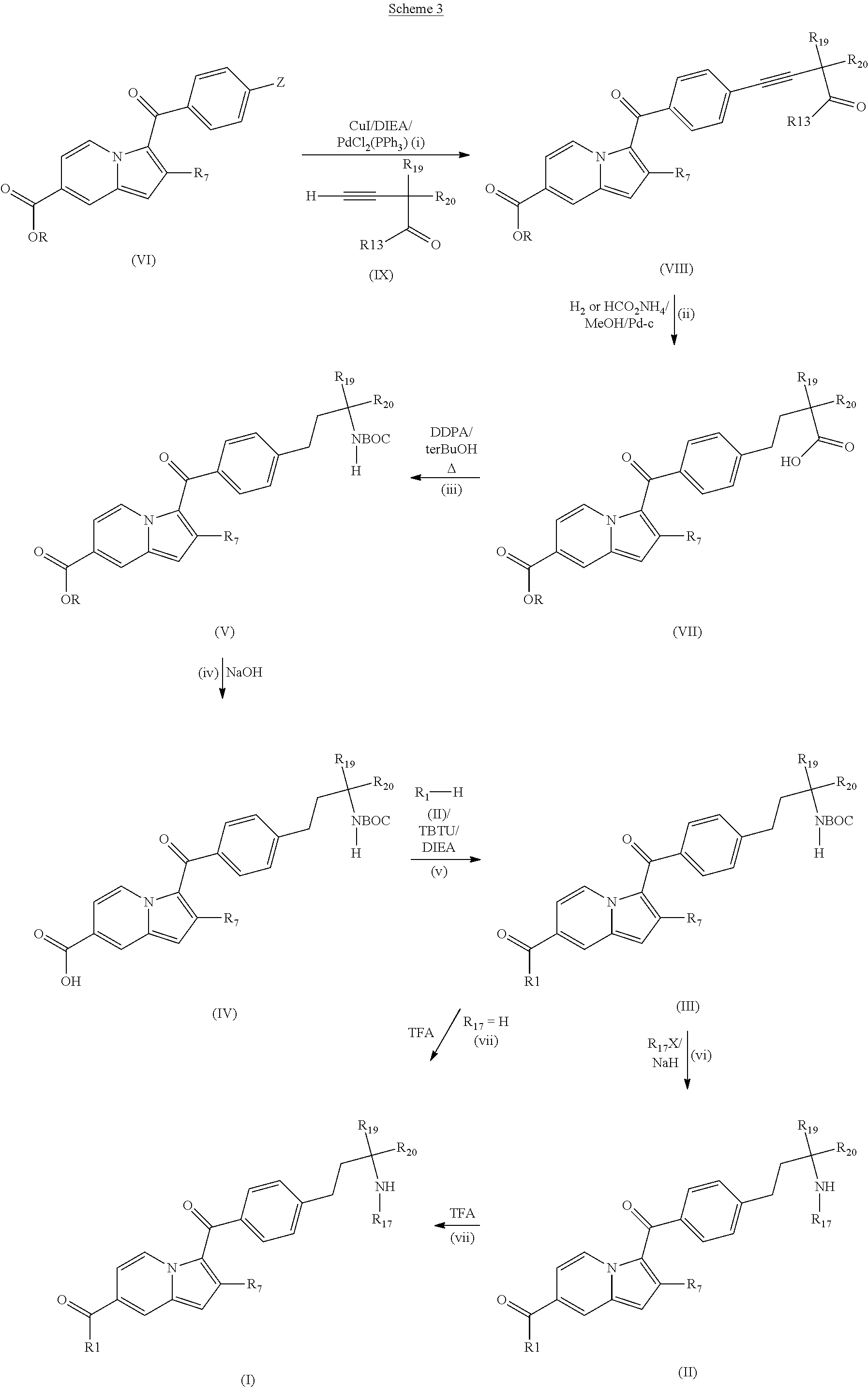

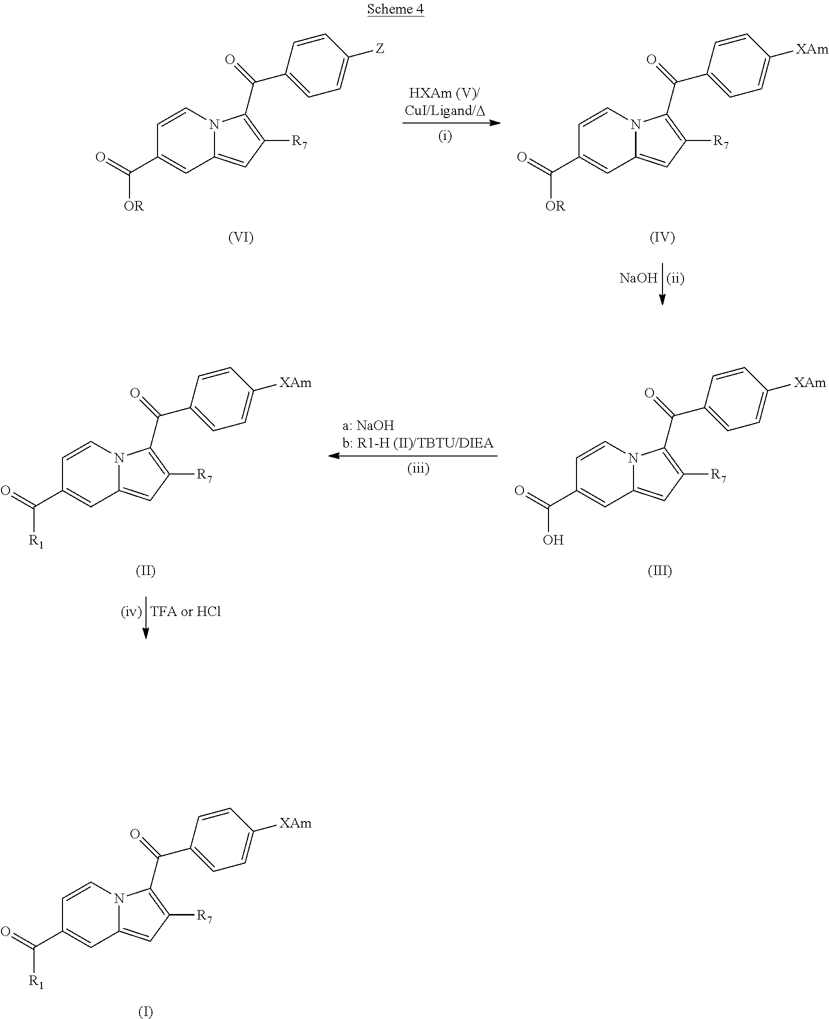

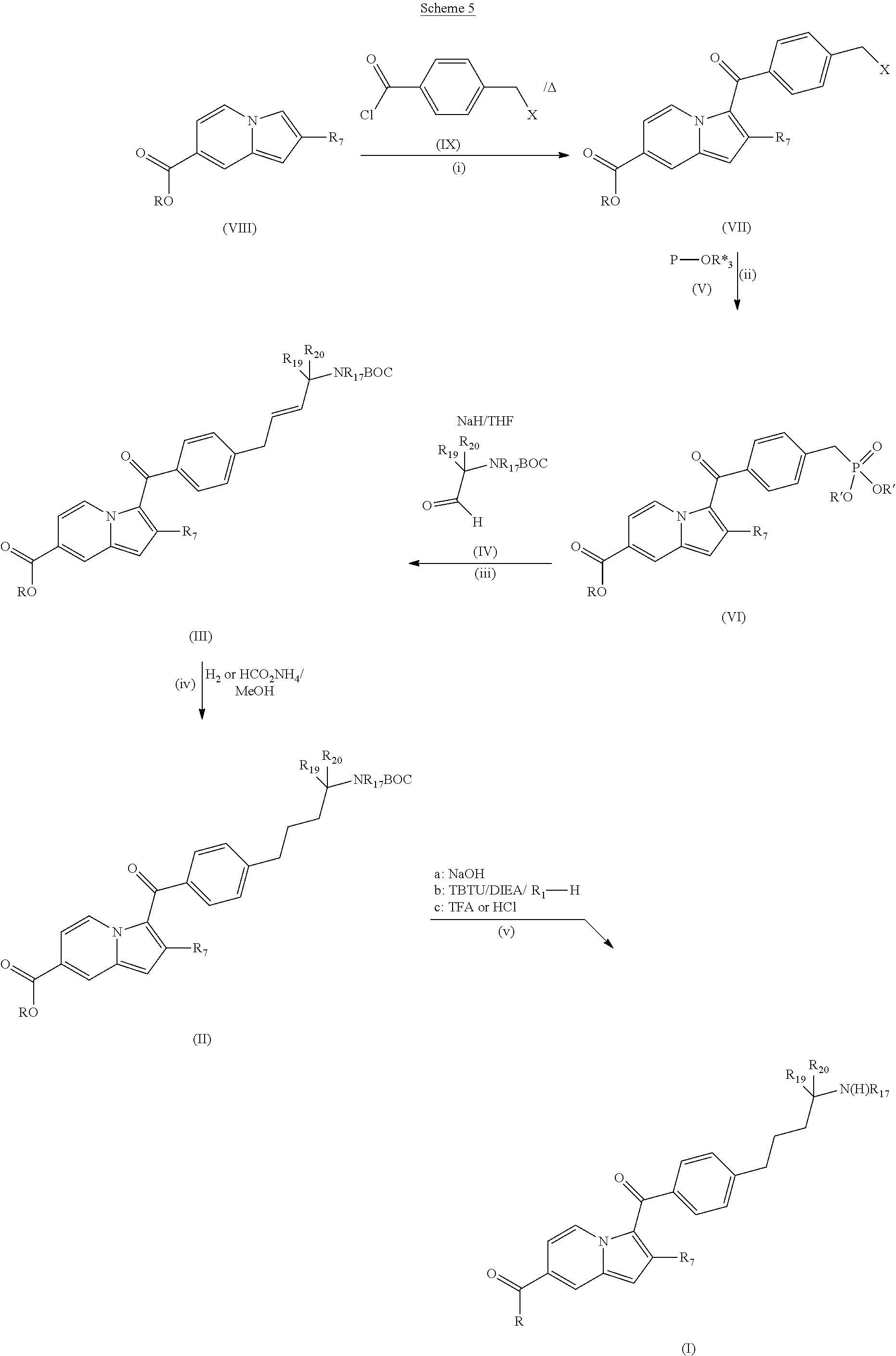

A subject of the invention, according to another of its aspects, is also the compounds of formula (VI)

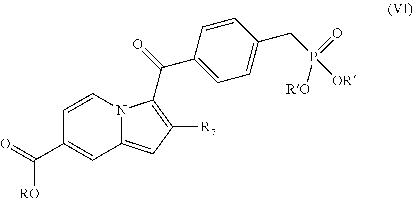

wherein:
R7 is as defined in claim 1;
R represents a (C₁-C₄) alkyl group;
R′ represents a (C₁-C₄) alkyl group;
P represents a phosphorus atom; in the form of a base or of an addition salt with an acid, as described in synthesis scheme 5. These compounds are useful as synthesis intermediates for the compounds of formula (I).

The following abbreviations and molecular formulae are used:
anh. anhydrous
EtOAc ethyl acetate
DCM dichloromethane
DCE dichloroethane
DIEA N,N-diisopropylethylamine
DIPA diisopropylamine
DIAD diisopropyl azodicarboxylate
DPPA diphenylphosphoryl azide
DMF dimethylformamide
EDCI N-ethyl-N′-(3-dimethylamino-propyl)carbodiimide*HCl
HMPA hexamethylphosphoramide
HOBt 1-hydroxybenzotriazole
HPLC high performance liquid chromatography
LC/MS liquid chromatography/mass spectrometry
NMP N-methylmorpholine
Pd—C palladium-on-carbon.
TBTU N-[(1H-benzotriazol-1-yloxy)(dimethylamino)methylidiene]-N-methylmethanaminium tetrafluoroborate
TEA triethylamine
THF tetrahydrofuran
AT ambient temperature
TFA trifluoroacetic acid
DIAD 1,1′-(azodicarbonyl)dipiperidine
DME dimethoxyethane
DMF dimethylformamide
DMSO dimethyl sulfoxide

The following examples illustrate the preparation of some compounds in accordance with the invention. The numbers of the compounds exemplified refer back to those of the table given later on which illustrates the chemical structures and the physical properties of some compounds according to the invention.

The melting points were measured with a “Büchi melting point B-545” instrument.

The optical rotations were measured with a “Perkin Elmer 343” instrument.

The mass spectra are obtained under the following LC/MS coupling conditions:

### Conditions A:

### Column: Kromasil 50×2.1 mm, 6.5 μm

### Eluents:

A=CH₃CN/TFA (0.05%)

B=H₂O/CH₃CN/TFA (1000:3:0.5)

### Gradients

| | | | Flow rate |
|---|---|---|---|
| t (mm) | % A | % B | (ml/mm) |
| 0 | 0 | 100 | 0.5 |
| 12 | 100 | 0 | 0.5 |
| 15 | 100 | 0 | 0.5 |

### Conditions B:

### Column: Acquity BEH C18 (50×2.1 mm, 1.7 μm)

### Eluents:

A=H₂O/TFA (0.05%)

B=CH₃CN/TFA (0.035%)

### Gradients

| | | | Flow rate |
|---|---|---|---|
| t (mm) | % A | % B | (ml/mm) |
| 0 | 98 | 2 | 1 |
| 1.6 | 0 | 100 | 1 |
| 2.1 | 0 | 100 | 1 |

The retention time is denoted Tr.
The proton magnetic resonance spectra (¹H NMR), as described below, are recorded at 400 MHz in DMSO-d6, using the DMSO-d5 peak as reference (δ=2.5 ppm). The chemical shifts δ are expressed in parts by million (ppm). The signals observed are expressed as follows: s=singlet; d=doublet; t=triplet; bs=unresolved peak or broad singlet; H=proton (for the rotamers, H_{M} and Hₘ are denoted with reference to the major and minor isomers M and m respectively).

### EXAMPLE 1

### Synthesis of the intermediate 1-methylethyl 2-butyl-3-({4-[3-(dibutylamino)propyl]phenyl}carbonyl)indolizine-7-carboxylate

### 1.1 1-methylethyl 2-methylpyridine-4-carboxylate

A mixture of 11.9 g (55.7 mmol) of 1-methylethyl 2-chloro-6-methylpyridine-4-carboxylate and 1.2 g of palladium-on-activated carbon at 10% in 150 ml of iPrOH is stirred for 24 h at ambient temperature under 4 bar of hydrogen. The term “ambient temperature” is intended to mean a temperature between 5 and 25° C. The reaction mixture is filtered and the filtrate is concentrated under reduced pressure. The residue obtained is then taken up with 200 ml of water, neutralized at 0° C. using Na₂CO₃, and then extracted with 3×200 ml of DCM. The organic phases are combined, dried over sodium sulfate, filtered, and then concentrated under reduced pressure.

The residue is purified by silica column chromatography, elution being carried out with an EtOAc/cyclohexane gradient of 0 to 30% with respect to EtOAc. After concentration under reduced pressure, 38.05 g of 1-methylethyl 2-methylpyridine-4-carboxylate are obtained in the form of a colorless oil. Yield=70%.

### 1. 2-Methyl-4-[(1-methylethoxy)carbonyl]-1-(2-oxohexyl)pyridinium bromide

A mixture of 9.88 g (55.13 mmol) of 1-methylethyl 2-methylpyridine-4-carboxylate and 14.88 g (82.69 mmol) of 1-bromohexan-2-one in 30 ml of butan-2-one is refluxed for 24 h. The reaction mixture is allowed to return to ambient temperature, and the resulting precipitate is filtered off and then washed successively with butan-2-one and pentane. 16.4 g of 2-methyl-4-[(1-methylethoxy)carbonyl]-1-(2-oxo-hexyl)pyridinium bromide are thus obtained in the form of a whitish powder which is used as it is in the next step.

Yield=82%.

### 1.3 1-Methylethyl 2-butylindolizine-7-carboxylate

A mixture of 16.4 g (45.52 mmol) of 2-methyl-4-[(1-methylethoxy)carbonyl]-1-(2-oxohexyl)pyridinium bromide and 14.17 g (136.52 mmol) of Na₂CO₃ in 200 ml of iPrOH is refluxed for 1 h 30. The reaction mixture is then concentrated under reduced pressure, and then taken up with 200 ml of water and extracted with 3×150 ml of DCM. The organic phases are combined, dried over the sodium sulfate, filtered, and then concentrated under reduced pressure. The residue obtained is purified by silica column chromatography, elution being carried out with DCM. After concentration under reduced pressure, 8.24 g of 1-methylethyl 2-butylindolizine-7-carboxylate are obtained in the form of a yellow solid.

Yield=70%.

### 1.4 1-methylethyl 2-butyl-3-{[4-(3-chloropropyl)phenyl]carbonyl}indolizine-7-carboxylate

8.24 g (31.77 mmol) of 1-methylethyl 2-butylindolizine-7-carboxylate and 6.89 g (31.77 mmol) of 4-(3-chloropropyl)benzoyl chloride are stirred for 4 h 30 at 85° C. At ambient temperature, the reaction mixture is taken up with 200 ml of water, neutralized with Na₂CO₃ and then extracted with 3×150 ml of ether. The organic phases are combined, dried over Na₂SO₄, filtered, and then concentrated under reduced pressure. The residue obtained is chromatographed on a silica column, elution being carried out with an EtOAc/cyclohexane gradient of 0 to 10% with respect to EtOAc. After concentration under reduced pressure, 12.4 g of 1-methylethyl 2-butyl-3-{[4-(3-chloro-propyl)phenyl]carbonyl}indolizine-7-carboxylate are obtained in the form of a yellow solid.

Yield=89%.

### 1.5 1-Methylethyl 2-butyl-3-({4-[3-(dibutylamino)propyl]phenyl}carbonyl)indolizine-7-carbxoylate hydrochloride

A mixture of 12.4 g (28.18 mmol) of 1-methylethyl 2-butyl-3-{[4-(3-chloropropyl)phenyl]carbonyl}indolizine-7-carboxylate, 5.46 g (42.27 mmol) of di-n-butylamine, 11.69 g (84.55 mmol) of K₂CO₃ and 4.68 g (28.18 mmol) of KI in 350 ml of CH₃CN is refluxed for 3 days. The reaction mixture is then concentrated under reduced pressure, taken up with 200 ml of water and then extracted with 3×200 ml of EtOAc. The organic phases are combined, dried over Na₂SO₄, filtered, and then concentrated under reduced pressure. The residue obtained is chromatographed on a silica column, elution being carried out with an EtOAc/cyclohexane gradient of 0 to 40% with respect to EtOAc. After concentration under reduced pressure, 12.7 g of 1-methylethyl 2-butyl-3-({4-[3-(dibutylamino)propyl]phenyl}carbonyl)indolizine-7-carboxylate are obtained in the form of a yellow oil.

Yield=85%.

The hydrochloride is prepared by taking up the base with a 0.1N solution of hydrochloric acid in iPrOH (1.1 eq.) which is then concentrated under reduced pressure, and the residue obtained is chromatographed on RP18 reverse phase, elution being carried out with a CH₃CN/H₂O (0.01N HCl) gradient of 0 to 100% with respect to CH₃CN, and then lyophilized.

Mp (° C.)=hygroscopic gum

LC/MS: M=C₃₄H₄₈N₂O₃=532; M+H=533; Tr 13.0 min (conditions A).

¹H NMR (ppm, d6-DMSO, 400 MHz):

10.30-10.15 (bs, 1H); 9.30 (d, 1H); 8.30 (s, 1H); 7.60 (d, 2H); 7.45 (d, 2H); 7.30 (d, 1H); 6.85 (s, 1H); 5.25-5.10 (bs, 1H); 3.15-2.95 (bs, 6H); 2.85-2.70 (t, 2H); 2.40-2.25 (t, 2H) 2.15-1.95 (bs, 2H) 1.70-1.55 (bs, 4H), 1.50-1.30 (bs, 12H); 1.10-1.00 (bs, 2H); 0.95 (t, 6H) 0.70 (6, 3H).

### EXAMPLE 2

### Compound No. 2: methyl (S)-1-{[2-butyl-3-({4-[3-(dibutylamino)propyl]phenyl}carbonyl)indolizin-7-yl]carbonyl}prolinate hydrochloride

### 2.1 2-Butyl-3-({4-[3-(dibutylamino)propyl]phenyl}carbonyl)indolizine-7-carboxylic acid

A mixture of 12.7 g (23.84 mmol) of 1-methylethyl 2-butyl-3-({4-[3-(dibutylamino)propyl]phenyl}carbonyl)indolizine-7-carboxylate and 1.91 g (47.68 mmol) of NaOH in 100 ml of dioxane and 20 ml of water is stirred for 3 days at ambient temperature. The reaction mixture is then neutralized with a 2N aqueous solution of hydrochloric acid, and concentrated under reduced pressure, and the resulting precipitate is filtered off, and washed with ice-cold water and then with ether. After drying under reduced pressure, 11.4 g of 2-butyl-3-({4-[3-dibutylamino)propyl]phenyl}carbonyl)indolizine-7-carboxylic acid are obtained in the form of a yellow solid.

Yield=97%.

### 2.2 Methyl (S)-1-{[2-butyl-3-({4-[3-(dibutyl-amino)propyl]phenyl}carbonyl)indolizin-7-yl]carbonyl}prolinate hydrochloride

1.0 g (3.06 mmol) of TBTU is added, in small amounts, at 0° C., under argon, to a solution of 1.5 g (3.06 mmol) of 2-butyl-3({4-[3-(dibutylamino)propyl]phenyl}carbonyl)indolizine-7-carboxylic acid, 0.51 g (3.06 mmol) of methyl (S)-prolinate and 1.07 ml (6.11 mmol) of DIEA in 30 ml of DCM. The reaction mixture is allowed to return slowly to ambient temperature and the stirring is continued for 18 h. The reaction mixture is taken up with 150 ml of DCM, washed successively with 2×75 ml of a saturated solution of NaHCO₃, 2×75 ml of water and 75 ml of brine, dried over Na₂SO₄ and filtered, and the filtrate is then treated with 1 ml of a 4N solution of hydrogen chloride in dioxane and then concentrated under reduced pressure. The residue obtained is chromatographed on RP18 reverse phase, elution being carried out with a CH₃CN/H₂O (0.01N HCl) gradient of 0 to 100% with respect to CH₃CN. After concentration under reduced pressure and lyophilization, 1.33 g of methyl (S)-1-{[2-butyl-3-({4-[3-(dibutylamino)propyl]phenyl}carbonyl)indolizin-7-yl]carbonyl}prolinate hydrochloride are obtained.

Yield=68%.

Mp (° C.): hygroscopic gum

[α]_{D}²⁰=−22 (c=0.1; MeOH)

LC/MS: M=C₃₇H₅₁N₃O₄=601; M+H=602; Tr=9.0 min (conditions A).

¹H NMR (ppm, d₆-DMSO, 400 MHz, 2 conformers M/m 8:2):

10.10-10.00 (bs, 1H); 9.35 (d, 1H); 7.90 (s, 1H_{M}); 7.70 (s, 1Hₘ); 7.60 (d, 2H); 7.40 (d, 2H); 7.05 (d, 1H_{M}); 6.85 (d, 1Hₘ); 6.70 (s, 1H_{M}); 6.65 (s, 1Hₘ); 4.70-4.60 (bs, 1Hₘ); 4.60-4.50 (bs, 1H_{M}); 3.80-3.45 (bs, 5H); 3.15-2.95 (bs, 6H); 2.80-2.70 (bs, 2H); 2.40-2.20 (bs, 3H); 2.10-1.85 (bs, 5H); 1.70-1.55 (bs, 4H); 1.45-1.25 (bs, 6H); 1.10-0.95 (bs, 2H); 0.90 (t, 6H); 0.70 (t, 3H).

### EXAMPLE 3

### Compound No. 3: methyl (R)-1-{[2-butyl-2-3-({4-[3-(dibutylamino)propyl]phenyl}carbonyl)indolizin-7-yl]carbonyl}prolinate hydrochloride

The process is carried out in the same way as in example 2.2. Thus, starting from 1.50 g (3.06 mmol) of 2-butyl-3-({4-[3-(dibutylamino)propyl]phenyl}carbonyl)indolizine-7-carboxylic acid and 0.50 g (3.06 mmol) of methyl (R)-prolinate, 1.20 g of methyl (R)-1-{[2-butyl-3-({4-[3-(dibutylamino)propyl]phenyl}carbonyl)indolizin-7-yl]carbonyl}prolinate hydrochloride are obtained after a reverse phase on RP18, elution being carried out with a CH₃CH/H₂O (0.01N HCl) gradient of 0 to 100% with respect to CH₃CN, and lyophilization.

Yield=65%.

Mp (° C.): hygroscopic gum

[α]_{D}²⁰=+22 (c=0.1; MeOH)

LC/MS: M=C₃₇H₅₁N₃O₄=6.01; M+H=602; Tr=9.0 min (conditions A).

¹H NMR (ppm, d₆-DMSO, 400 MHz, 2 conformers M/m 85:15): 9.90-9.75 (bs, 1H); 9.35 (d, 1H); 7.90 (s, 1H_{M}); 7.70 (s, 1Hₘ); 7.60 (d, 2H); 7.45 (d, 2H); 7.05 (d, 1H_{M}); 6.90 (d, 1Hₘ); 6.75 (s, 1H_{M}); 6.65 (s, 1Hₘ); 4.75-4.65 (bs, 1Hₘ); 4.60-4.50 (bs, 1H_{M}); 3.80-3.50 (bs, 5H); 3.15-2.95 (bs, 6H); 2.85-2.70 (bs, 2H); 2.40-2.20 (bs, 3H); 2.10-1.80 (bs, 5H); 1.70-1.50 (bs, 4H); 1.45-1.25 (bs, 6H); 1.10-0.95 (bs, 2H); 0.90 (t, 6H); 0.70 (t, 3H).

### EXAMPLE 4

### Compound No. 4: 2-butyl-3-({4-[3-(dibutylamino)propyl]phenyl}carbonyl)-N-ethyl-N-(2-methoxyethyl)indolizine-7-carboxamide hydrochloride

The process is carried out in the same way as in example 2.2. Thus, starting from 1.0 g (2.04 mmol) of 2-butyl-3-({4-[3-(dibutylamino)propyl]phenyl}carbonyl)indolizine-7-carboxylic acid and 0.36 g (2.55 mmol) of N-ethyl-2-methoxyethanamine, 0.75 g of 2-butyl-3-({4-[3-(dibutylamino)propyl]phenyl}carbonyl)-N-ethyl-N-(2-methoxyethyl)indolizine-7-carboxamide hydrochloride are obtained in the form of a hygroscopic yellow foam after an RP18 reverse phase and lyophilization.

Yield=60%.

Mp (° C.): hygroscopic gum

LC/MS: M=C₃₆H₅₃N₃O₃=575; M+H=576; Tr=9.6 min (conditions A)

¹H NMR (ppm, d6-DMSO, 400 MHz):

10.00 (sl, 1H); 9.40 (d, 1H); 7.70 (s, 1H); 7.60 (d, 2H); 7.45 (d, 2H); 6.90 (d, 1H); 6.65 (s, 1H); 3.70-3.15 (bs, 8H); 3.15-2.95 (bs, 6H); 2.80-2.70 (bs, 2H); 2.30-2.20 (bs, 2H); 2.10-1.95 (bs, 2H); 1.70-1.55 (bs, 4H); 1.40-1.25 (bs, 6H); 1.20-0.95 (bs, 6H); 0.90 (t, 6H); 0.70 (t, 3H).

### EXAMPLE 5

### Compound No. 5: methyl N-{[2-butyl-3-({4-[3-(dibutylamino)propyl]phenyl}carbonyl)indolizin-7-yl]carbonyl}-N-ethylglycinate hydrochloride

### 5.1 methyl N-ethylglycinate hydrochloride

3 ml (40.72 mmol) of thionyl chloride are added dropwise, at 0° C., to a solution of 2.1 g (20.36 mmol) of N-ethylglycine in 40 ml of MeOH. The reaction mixture is allowed to return to ambient temperature and then, after stirring for 4 h, the reaction mixture is concentrated under reduced pressure. The residue obtained is solidified from ether, filtered and washed successively with ether and pentane. 3 g of methyl N-ethylglycinate hydrochloride are obtained in the form of a white solid which is used as it is in the next step.

Yield=95%.

### 5.2 Methyl N-{[2-butyl-3-({4-[3-(dibutylamino)propyl]phenyl}carbonyl)indolizin-7-yl]carbonyl}-N-ethylglycinate hydrochloride

The process is carried out in the same way as in example 2.2. Thus, starting from 2.0 g (4.08 mmol) of 2-butyl-3-({4-[3-(dibutylamino)propyl]phenyl}carbonyl)indolizine-7-carboxylic acid and 0.78 g (5.09 mmol) of N-ethylglycinate hydrochloride, 0.77 g of methyl N-{[2-butyl-3-({4-[3-(dibutylamino)propyl]phenyl}carbonyl)indolizin-7-yl]carbonyl}-N-ethylglycinate hydrochloride is obtained in the form of a hygroscopic yellow foam, after an RP18 reverse phase, elution being carried out with a CH₃CN/H₂O (0.01N HCl) gradient of 0 to 30% with respect to CH₃CN, and lyophilization.

Yield=30%.

Mp (° C.): hygroscopic gum

LC/MS: M=C₃₆H₅₁N₃O₄=589; M+H=590; Tr=9.30 min (conditions A)

¹H NMR (ppm, d₆-DMSO, 400 MHz, 2 conformers):

10.15-10.00 (bs, 1H); 9.45-9.35 (bs, 1H); 7.75-7.65 (bs, 1H); 7.60 (d, 2H); 7.45 (d, 2H); 7.00-6.85 (bs, 1H); 6.75-6.65 (bs, 1H); 4.25 (s, 2H); 3.80-3.65 (bs, 3H); 3.55-3.35 (bs, 3H); 3.15-2.95 (bs, 6H); 2.85-2.70 (t, 2H); 2.30-2.20 (t, 2H); 2.10-1.95 (bs, 2H); 1.70-1.55 (bs, 4H); 1.45-1.25 (bs, 6H); 1.20-1.10 (t, 2H); 1.10-1.00 (bs, 2H); 0.95 (t, 6H); 0.70 (t, 3H).

### EXAMPLE 6

### Compound No. 6: N-{[2-butyl-3-({4-[3-(dibutylamino)propyl]phenyl}carbonyl)indolizin-7-yl]carbonyl}-N-ethylglycine hydrochloride

3.0 ml (3.0 mmol) of a 1N aqueous solution of sodium hydroxide are added, at 0° C., to a solution of 1.6 g (2.71 mmol) of methyl N-{[2-butyl-3-({4-[3-(dibutylamino)propyl]phenyl}carbonyl)indolizin-7-yl]carbonyl}-N-ethylglycinate, and then the reaction mixture is allowed to return to ambient temperature and the stirring is continued for 24 h. The reaction mixture is treated with 1.0 ml of a 4N solution of hydrogen chloride in dioxane, concentrated under reduced pressure, and then chromatographed on RP18 reverse phase, elution being carried out with a CH₃CH/H₂O (0.01N HCl) gradient of 0 to 30% with respect to CH₃CN. After concentration under reduced pressure and lyophilization, 0.78 g of N-{[2-butyl-3-({4-[3-(dibutylamino)propyl]phenyl}carbonyl)indolizin-7-yl]carbonyl}-N-ethylglycine hydrochloride is obtained in the form of a hygroscopic yellow foam.

Yield=41%.

Mp (° C.): hygroscopic yellow foam

LC/MS: M=C₃₅H₄₉N₃O₄=575; M+H=576; Tr=8.6 mins (conditions A)

¹H NMR (ppm, d6-DMSO, 400 MHz):

9.45-9.30 (bs, 1H); 7.75-7.55 (bs, 3H); 7.4 (d, 2H); 6.95-6.80 (bs, 1H); 6.75-6.60 (bs, 1H); 4.20-4.05 (bs, 2H); 3.60-3.20 (bs, 2H); 3.15-2.95 (bs, 6H); 2.85-2.70 (t, 2H); 2.30-2.20 (t, 2H); 2.10-1.95 (bs, 2H); 1.70-1.55 (bs, 4H); 1.45-1.25 (bs, 6H); 1.20-0.90 (bs, 11H); 0.70 (t, 3H).

### EXAMPLE 7

### Compound No. 7: 3-({2-butyl-3[4-(3-dibutylaminopropyl)benzoyl]indolizine-7-carbonyl}ethylamino)propionic acid hydrochloride

### 7.1 Methyl N-{[2-butyl-3-({4-[3-(dibutylamino)propyl]phenyl}carbonyl)indolizin-7-yl]carbonyl}-N-ethyl-β-alaninate hydrochloride

The process is carried out in the same way as in example 2.2. Thus, starting from 2.0 g (4.08 mmol) of 2-butyl-3-({4-[3-(dibutylamino)propyl]phenyl}carbonyl)-N,N-diethylindolizine-7-carboxamide acid and 0.54 g (4.08 mmol) of methyl N-ethyl-β-alaninate, 2.2 g of methyl N-{[2-butyl-3-({4-[3-(dibutylamino)propyl]phenyl}carbonyl)indolizin-7-yl]carbonyl}-N-ethyl-β-alaninate hydrochloride are obtained.

Yield=89%.

MP (° C.): hygroscopic yellow foam

LC/MS: M=C₃₇H₅₃N₃O₄=603; M+H=604; Tr=1.18 min (conditions B)

¹H NMR (ppm, d₆-DMSO, 400 MHz):

9.40 (d, 1H); 7.65 (s, 1H); 7.60 (d, 2H); 7.45 (d, 2H); 6.90 (d, 1H), 6.65 (s, 1H); 3.80-3.20 (bs, 7H); 3.15-3.00 (bs, 6H); 2.80-2.70 (bs, 2H); 2.70-2.60 (t, 2H); 2.30-2.20 (t, 2H); 2.10-1.95 (bs, 2H) 1.70-1.55 (bs, 4H); 1.45-1.25 (bs, 6H); 1.20-0.90 (bs, 11H); 0.70 (t, 3H).

### 7.2 3-({2-Butyl-3-[4-(3-dibutylaminopropyl)benzoyl]indolizine-7-carbonyl}ethylamino)propionic acid hydrochloride

The process is carried out in the same way as in example 6. Thus, starting from 1.1 g (1.82 mmol) of methyl N-{[2-butyl-3-({4-[3-(dibutylamino)propyl]phenyl}carbonyl)indolizin-7-yl]carbonyl}-N-ethyl-β-alaninate, 0.91 g of 3-({2-butyl-3-[4-(3-dibutylamino-propyl)benzoyl]indolizine-7-carbonyl}ethylamino)propionic acid hydrochloride is obtained in the form of a hygroscopic foam.

Yield=85%.

Mp (° C.): hygroscopic foam

LC/MS: M=C₃₆H₅₁N₃O₄=589; M+H=590; Tr=1.09 min (conditions B)

¹H NMR (ppm, d₆-DMSO, 400 MHz):

9.40 (d, 1H); 7.65 (s, 1H); 7.60 (d, 2H); 7.45 (d, 2H); 6.90 (d, 1H), 6.65 (s, 1H); 3.80-3.20 (bs, 4H); 3.15-3.00 (bs, 6H); 2.80-2.70 (bs, 2H); 2.70-2.60 (t, 2H); 2.30-2.20 (t, 2H); 2.10-1.95 (bs, 2H) 1.70-1.55 (bs, 4H); 1.45-1.25 (bs, 6H); 1.20-0.90 (bs, 11H); 0.70 (t, 3H).

### EXAMPLE 8

### Compound No. 8: methyl N-{[3-({4-[3-(cyclopentylamino)propyl]phenyl}carbonyl)-2-ethylindolizin-7-yl]carbonyl}-N-propane-2-ylglycinate hydrochloride

### 8.1 2-Methyl-1-(2-oxopropyl)-4-[(propan-2-yloxy)carbonyl]pyridinium bromide

The process is carried out in the same way as in example 1.2. Thus, starting from 39.0 g (217.61 mmol) of 1-methylethyl 2-methylpyridine-4-carboxylate and 49.29 g (326.42 mmol) of 1-bromobutanone in 120 ml of butan-2-one, 66.15 g of 2-methyl-1-(2-oxopropyl)-4-[(propan-2-yloxy)carbonyl]pyridinium bromide are obtained in the form of a pale yellow powder which is used as it is in the next step.

Yield=96%.

### 8.2 Propan-2-yl 2-ethylindolizine-7-carboxylate

The process is carried out in the same way as in example 1.3. Thus, starting from 66.15 g (209 mmol) of 2-methyl-1-(2-oxopropyl)-4-[(propan-2-yloxy)carbonyl]pyridinium bromide and 6.5 g (627.62 mmol) of Na₂CO₃ in 700 ml of iPrOH, 40 g of propan-2-yl 2-ethylindolizine-7-carboxylate are obtained in the form of a pale yellow solid.

Yield=83%.

### 8.3 Propan-2-yl 3-{[4-(3-chloropropyl)phenyl]carbonyl}-2-ethylindolizine-7-carboxylate

The process is carried out in the same way as in example 1.4. Thus, starting from 10.0 g (43.24 mmol) of propan-2-yl 2-ethylindolizine-7-carboxylate and 25.03 g (51.88 mmol) of 4-(3-chloropropyl)benzoyl chloride, 17.6 g of propan-2-yl 3-{[4-(3-chloropropyl)phenyl]carbonyl}-2-ethylindolizine-7-carboxylate are obtained in the form of a yellowish oil which crystallizes slowly.

Yield=98%.

### 8.4 Propan-2-yl 3-[(4-{3-[(tert-butoxycarbonyl)(cyclo-pentyl)amino]propyl}phenyl)carbonyl]-2-ethylindolizine-7-carboxylate

In a sealed tube, a mixture of 4.0 g (9.71 mmol) of propan-2-yl 3-{[4-(3-chloropropyl)phenyl]carbonyl}-2-ethylindolizine-7-carboxylate, 1.65 g (19.42 mmol) of cyclopentylamine and 1.69 g (10.2 mmol) of KI in 30 ml of a 2:1 CH₃CN/DMF mixture is heated for 18 h at 105° C. The reaction mixture is then concentrated under reduced pressure, and then taken up with 200 ml of DCM, washed successively with 2×300 ml of water and 100 ml of brine, dried over Na₂SO₄, filtered, and concentrated under reduced pressure. 2.08 g of a yellow powder are thus obtained, and taken up with 25 ml of DCM, and then 1.28 g (5.90 mmol) of Boc₂O and 0.46 g (4.52 mmol) of TEA are added at 0° C. After stirring for 18 h at ambient temperature (AT), the reaction mixture is taken up with 200 ml of DCM, washed successively with 100 ml of water and 100 ml of brine, then dried over MgSO₄, filtered, and concentrated under reduced pressure. The residue obtained is purified by silica gel chromatography, elution being carried out with a DCM/MeOH gradient of 0 to 5% with respect to MeOH. After concentration under reduced pressure, 2.37 g of propan-2-yl 3-[(4-{3-[(tert-butoxycarbonyl)(cyclo-pentyl)amino]propyl}phenyl)carbonyl]-2-ethylindolizine-7-carboxylate are obtained in the form of a yellow gum.

Yield=43%.

### 8.5 3-[(4-{3-[(tert-Butoxycarbonyl)(cyclopentyl)amino]propyl}phenyl)carbonyl]-2-ethylindolizine-7-carboxylic acid

8.5 ml of a 1N aqueous NaOH solution are added dropwise, at AT, to a solution of 2.37 g (4.23 mmol) of propan-2-yl 3-[(4-{3-[(tert-butoxycarbonyl)(cyclopentyl)amino]propyl}phenyl)carbonyl]2-ethylindolizine-7-carboxylate in 10 ml of dioxane and the stirring is continued for 72 h. The reaction mixture is cooled to 0° C., treated by adding, dropwise, 10 ml of a 1N aqueous HCl solution and then extracted with 2×200 ml of DCM. The organic phases are combined, washed with 100 ml of brine, dried over Na₂SO₄, filtered, and then concentrated under reduced pressure. 2.29 g of 3-[(4-{3-[(tert-butoxycarbonyl)(cyclopentyl)amino]propyl}phenyl)carbonyl]-2-ethylindolizine-7-carboxylic acid are thus obtained in the form of a yellow solid which is used as it is in the next step.

Yield=100%.

### 8.6 Methyl N-({3-[(4-{3-[(tert-butoxycarbonyl)(cyclo-pentyl)amino]propyl}phenyl)carbonyl]-2-ethylindolizin-7-yl}carbonyl)-N-propan-2-ylglycinate

With the exception of the salification step, the process is carried out in the same way as in example 2.2. Thus, starting from 2.29 g (4.42 mmol) of 3-[(4-{3-[(tert-butoxycarbonyl)(cyclopentyl)amino]propyl}phenyl)carbonyl]-2-ethylindolizine-7-carboxylic acid, 0.96 g (5.74 mmol) of methyl N-propan-2-ylglycinate hydrochloride, 1.71 g (13.25 mmol) of DIEA and 2.13 g (6.62 mmol) of TBTU in 20 ml of DCM, 2.0 g of methyl N-({3-[(4-{3-[(tert-butoxycarbonyl)(cyclo-pentyl)amino]propyl}phenyl)carbonyl]-2-ethylindolizin-7-yl}carbonyl)-N-propan-2-ylglycinate are obtained in the form of a yellow foam, after purification on a silica column, elution being carried out with an EtOAc/cyclohexane gradient of 0 to 40% of EtOAc.

Yield=72%.

### 8.7 Methyl N-{[3-({4-[3-(cyclopentylamino)propyl]phenyl}carbonyl)-2-ethylindolizine-7-yl]carbonyl}-N-propan-2-ylglycinate hydrochloride

A 2N solution of hydrogen chloride in Et₂O is added dropwise, at 0° C., to a solution of 2.0 g (3.06 mmol) of methyl N-({3-[(4-{3-[(tert-butoxycarbonyl)(cyclopentyl)amino]propyl}phenyl)carbonyl]-2-ethylindolizin-7-yl}carbonyl)-N-propan-2-ylglycinate in 20 ml of DCM and then the reaction mixture is allowed to return to AT. After stirring for 24 h, the reaction mixture is concentrated under reduced pressure and the residue obtained is triturated from Et₂O, filtered through a sintered glass funnel and washed with Et₂O and then dried under reduced pressure. 1.72 g of methyl N-{[3-({4-[3-(cyclopentylamino)propyl]phenyl}carbonyl)-2-ethylindolizine-7-yl]carbonyl}-N-propan-2-ylglycinate hydrochloride are thus obtained in the form of a yellow powder.

Yield=96%.

Mp (° C.): 228

LC/MS: M=C₃₂H₄₁N₃O₄=531; M+H=532; Tr=1.09 min (conditions B).

¹H NMR (ppm, d6-DMSO, 400 MHz):

9.60-9.50 (bs, 1H); 8.70-8.50 (bs, 1H); 7.70 (s, 1H); 7.60 (d, 2H); 7.40 (d, 2H); 6.90 (d, 1H); 6.70 (s, 1H); 4.20-4.00 (bs, 3H); 3.75-3.65 (bs, 3H); 3.50-3.40 (bs, 1H); 3.00-2.90 (t, 2H); 2.90-2.80 (t, 2H); 2.30-2.20 (bs, 2H); 2.10-1.95 (bs, 4H); 1.80-1.70 (bs, 2H); 1.70-1.50 (bs, 4H); 1.20-1.05 (bs, 6H); 1.00 (t, 3H).

### EXAMPLE 9

### Compound No. 9: 2-butyl-3-({4-[3-(dibutylamino)propyl]phenyl}carbonyl)-N-ethyl-N-[(2-methyl-2H-tetrazol-5-yl)methyl]indolizine-7-carboxamide hydrochloride

### 9.1 tert-Butyl {2-[(2-cyanoethyl)amino]-2-oxoeth-yl}ethylcarbamate

11.31 g (59.04 mmol) of EDCI are added, at 0° C. and in small amounts, to a mixture of 10.0 g (49.2 mmol) of N-(tert-buytoxycarbonyl)-N-ethylglycine, 6.89 g (98.41 mmol) of 3-aminopropanenitrile and 7.53 g (49.20 mmol) of HOBT in 230 ml of an 8:2 DCM/THF mixture, and then the reaction mixture is allowed to return slowly to AT and the stirring is continued for 18 h. The reaction mixture is washed successively with 2×100 ml of water and 2×100 ml of a saturated solution of K₂CO₃, dried over MgSO₄, filtered, and then concentrated under reduced pressure. 11.0 g of tert-butyl {2-[(2-cyanoethyl)amino]-2-oxoethyl}ethylcarbamate are thus obtained in the form of a white solid which is used as it is in the next step.

Yield=88%

### 9.2 tert-butyl {[1-(2-cyanoethyl)-1H-tetrazol-5-yl]methyl}ethylcarbamate

12.32 g (47.00 mmol) of PPh₃ and then 9.36 ml (70.50 mmol) of trimethylsilyl azide are added under argon, in small amounts, to a mixture of 6.0 g (23.50 mmol) of tert-butyl {2-[(2-cyanoethyl)amino]-2-oxoethyl}ethylcarbamate and 9.5 g (47.00 mmol) of DIAD in 48 ml of anh. THF. After stirring at AT for 48 h, the reaction mixture is taken up with 250 ml of EtOAc, washed successively with 2×100 ml of water and 2×100 ml of brine, dried over MgSO₄, filtered, and then concentrated under reduced pressure. The residue obtained is purified by silica gel column chromatography, elution being carried out with an EtOAc/cyclohexane gradient of 0 to 40% with respect to EtOAc. After concentration under reduced pressure, 3.2 g of tert-butyl {[1-(2-cyanoethyl)-1H-tetrazol-5-yl]methyl}ethylcarbamate are obtained in the form of a reddish oil.

Yield=48%.

### 9.3 tert-Butyl ethyl(1H-tetrazol-5-ylmethyl)carbamate

A mixture of 3.3 g (11.77 mmol) of tert-butyl {[1-(2-cyanoethyl)-1H-tetrazol-5-yl]methyl}ethylcarbamate and 17.7 ml of a 1N aqueous NaOH solution in 24.0 ml of THF is stirred for 3 days at AT. The reaction mixture is then cooled to 0° C., neutralized by adding, dropwise, 17.7 ml of a 1N aqueous HCl solution and then extracted with 2×150 ml of DCM after addition of 40 ml of brine. The organic phases are combined, washed with 50 ml of brine, dried over Na₂SO₄, filtered, and then concentrated under reduced pressure. 2.76 g of tart-butyl ethyl(1H-tetrazol-5-ylmethyl)carbamate are then obtained in the form of a colorless oil which is used as it is in the next step.

Yield=51%.

### 9.4 tert-Butyl ethyl[(2-methyl-2H-tetrazol-5-yl)methyl]carbamate

0.517 g (12.92 mmol) of NaH at 60% in oil is added in small amounts, at 0° C., under argon, to a solution of 2.67 g (11.75 mmol) of tert-butyl ethyl(1H-tetrazol-5-ylmethyl)carbamate in 10.7 ml of anh. DMF. After stirring for 30 minutes at 0° C., 0.73 ml (11.75 mmol) of iodomethane is added dropwise and the stirring is continued for 18 h at AT. The reaction mixture is taken up with 150 ml of EtOAc, washed successively with 2×100 ml of water and 100 ml of brine, dried over Na₂SO₄, filtered, and then concentrated under reduced pressure. The residue obtained is purified by silica column chromatography, elution being carried out with an EtOAc/cyclohexane gradient of 0 to 40% with respect to EtOAc. After concentration under reduced pressure, 0.95 g of tert-butyl ethyl[(2-methyl-2H-tetrazol-5-yl)methyl]carbamate and 0.76 g of tert-butyl ethyl[(2-methyl-2H-tetrazol-5-yl)methyl]carbamate are isolated in the form of colorless oils.

Yield=60%.

### 9.5 N-[(2-methyl-2H-tetrazol-5-yl)methyl]ethanamine hydrochloride

6 ml of a 2N solution of hydrogen chloride in Et₂O are added to a solution of 0.95 g (3.17 mmol) of tert-butyl ethyl[(1-methyl-1H-tetrazol-5-yl)methyl]carbamate in 6 ml of DCM and the stirring is continued for 18 h at AT. The reaction mixture is then concentrated under reduced pressure, triturated from Et₂O, filtered, and dried under reduced pressure. 0.395 g of N-[(2-methyl-2H-tetrazol-5-yl)methyl]ethanamine hydrochloride is thus obtained in the form of a white solid which is used as it is in the next step.

Yield=56%.

### 9.6 2-Butyl-3-({4-[3-(dibutylamino)propyl]phenyl}carbonyl)-N-ethyl-N-[(2-methyl-2H-tetrazol-5-yl)methyl]indolizine-7-carboxide hydrochloride

The process is carried out in the same way as in example 2.2. Thus, starting from 0.93 g (1.90 mmol) of 2-butyl-3-({4-[3-(dibutylamino)propyl]phenyl}carbonyl)indolizine-7-carboxylic acid, 0.36 g (2.0 mmol) of N-[(1-methyl-1H-tetrazol-5-yl)methyl]ethanamine hydrochloride, 0.74 g (5.70 mmol) of DIEA and 0.92 g (2.85 mmol) of TBTU in 9.5 ml of DCM, 0.91 g of 2-butyl-3-({4-[3-(dibutylamino)propyl]phenyl}carbonyl)-N-ethyl-N-[(2-methyl-2H-tetrazol-5-yl)methyl]indolizine-7-carboxamide hydrochloride is obtained in the form of a hygroscopic white powder.

Yield=73%.

LC/MS: M=C₃₆H₅₁N₇O₂=613; M+H=614; Tr=1.16 min (conditions B)

¹H NMR (ppm, d₆-DMSO, 400 MHz):

10.40-10.30 (bs, 1H); 9.40 (d, 1H); 7.80-7.70 (bs, 1H); 7.60 (d, 2H); 7.50 (d, 2H); 7.00-6.90 (bs, 1H); 7.70 (s, 1H); 5.00-4.80 (bs, 2H); 4.40 (s, 3H); 3.55-3.40 (bs, 2H); 3.15-3.00 (bs, 6H); 2.80-2.70 (t, 2H); 2.30-2.20 (t, 2H); 2.10-1.95 (bs, 2H); 1.70-1.60 (bs, 4H); 1.45-1.25 (bs, 6H); 1.20-1.10 (t, 2H); 1.10-1.00 (bs, 2H); 1.00 (t, 6H); 0.70 (t, 3H).

### EXAMPLE 10

### Compound No. 10: 2-butyl-3-({4-[3-(dibutylamino)propyl]phenyl}carbonyl)-N-ethyl-N-[(3-methyl-1,2,4-oxodiazol-5-yl)methyl]indolizine-7-carboxamide hydrochloride

### 10.1 tert-Butyl ethyl[(3-methyl-1,2,4-oxadizol-5-yl)-methyl]carbamate

1.9 g (29.92 mmol) of NaH at 60% in oil are added in small amounts, at 0° C. under argon, to a mixture of 1.3 g (17.149 mmol) of N′-hydroxyethanimidamide and 3 g of powdered 3 Å molecular sieve in 184 ml of anh. THF. After stirring for 1 h at AT, a solution of 2.0 g (9.21 mmol) of methyl N-(tert-butoxycarbonyl)-N-ethylglycinate in 30 ml of anh. THF is added and then the reaction mixture is refluxed for 18 h. The mixture is then filtered, concentrated under reduced pressure, taken up with 200 ml of DCM, washed successively with 2×100 ml of water and 100 ml of brine, dried over Na₂SO₄, filtered, and then again concentrated under reduced pressure. The residue obtained is purified by silica column chromatography, elution being carried out with an EtOAc/cyclohexane mixture of 0 to 40% with respect to EtOAc. After concentration under reduced pressure, 1.65 g of Cert-butyl ethyl[(3-methyl-1,2,4-oxadiazol-5-yl)methyl]carbamate are obtained in the form of a colorless oil.

Yield=74%.

### 10.2 N-[(3-Methyl-1,2,4-oxadiazol-5-yl)methyl]ethanamine hydrochloride

The process is carried out in the same way as in example 9.5. Thus, starting from 1.65 g (6.85 mmol) of tert-butyl ethyl[(3-methyl-1,2,4-oxadiazol-5-yl)methyl]carbamate, 1.05 g of N-[(3-methyl-1,2,4-oxadiazol-5-yl)methyl]ethanamine hydrochloride are obtained in the form of a white powder.

Yield=86%.

### 10.3 2-Butyl-3-({4-[3-(dibutylamino)propyl]phenyl}carbonyl)-N-ethyl-N-[(3-methyl-1,2,4-oxadiazol-5-yl)methyl]indolizine-7-carboxamide hydrochloride

The process is carried out in the same way as in example 2.2. Thus, starting from 0.52 g (2.91 mmol) of N-[(3-methyl-1,2,4-oxadiazol-5-yl)methyl]ethanamine hydrochloride, 1.3 g (2.65 mmol) of 2-butyl-3-({4-[3-(dibutylamino)propyl]phenyl}carbonyl)indolizine-7-carboxylic acid and 1.03 g (3.97 mmol) of TBTU in 1.4 ml of DCM, 1.04 g of 2-butyl-3-({4-[3-(dibutylamino)propyl]phenyl}carbonyl)-N-ethyl-N-[(3-methyl-1,2,4-oxadiazol-5-yl)methyl]indolizine-7-carboxamide hydrochloride are obtained in the form of a gum.

Yield=58%.

LC/MS: M=C₃₇H₅₁N₅O₃=613; M+H=614; Tr=1.18 min (conditions B)

¹H NMR (ppm, d₆-DMSO, 400 MHz):

10.35-10.20 (bs, 1H); 9.4 (d, 1H); 7.75 (s, 1H); 7.60 (d, 2H); 7.40 (d, 2H); 6.95 (d, 1H); 6.70 (s, 1H); 4.90 (s, 2H); 3.60-3.45 (bs, 2H); 3.15-3.00 (bs, 6H); 2.85-2.75 (t, 2H); 2.40 (s, 3H); 2.30-2.20 (t, 2H); 2.10-1.95 (bs, 2H); 1.7-1.55 (bs, 4H); 1.40-1.25 (bs, 6H); 1.20 (t, 3H); 1.10-0.95 (bs, 2H); 0.90 (t, 6H); 0.70 (t, 3H).

### EXAMPLE 11

### Compound No. 11: 2-butyl-3-({4-[3-(dibutyl-amino)propyl]phenyl}carbonyl)-N-ethyl-N-(1H-tetrazol-5-ylmethyl)indolizine-7-carboxamide hydrochloride

### 11.1 3-{5-[(Ethylamino)methyl]-1H-tetrazol-1-yl}propanenitrile hydrochloride

The process is carried out in the same way as in example 9.5. Thus, starting from 3.02 g (11.42 mmol) of tert-butyl {[1-(2-cyanoethyl)-1H-tetrazol-5-yl]methyl}-ethylcarbamate, 1.83 g of 3-{5-[(ethylamino)methyl]-1H-tetrazol-1-yl}propanenitrile hydrochloride are obtained in the form of a white powder.

Yield=74%.

### 11.2 2-Butyl-N-{([1-(2-cyanoethyl)-1H-tetrazol-5-yl]methyl}-3-({4-[3-(dibutylamino)propyl]phenyl}carbonyl)-N-ethylindolizine-7-carboxamide

With the exception of the salification step, the process is carried out in the same way as in example 2.2. Thus, starting from 2.0 g (4.08 mmol) of 2-butyl-3-({4-[3-(dibutylamino)propyl]phenyl}carbonyl)indolizine-7-carboxylic acid, 0.97 g (4.48 mmol) of 3-{5-[(ethylamino)methyl]-1H-tetrazol-1-yl}propanenitrile hydrochloride, 1.58 g (12.23 mmol) of DIEA and 1.97 g (6.11 mmol) of TBTU in 20 ml of DCM and with purification on a silica column, elution being carried out with a DCM/MeOH gradient of 0 to 5% with respect to MeOH, after concentration under reduced pressure, 1.35 g of 2-butyl-N-{[1-(2-cyanoethyl)-1H-tetrazol-5-yl]methyl}-3-({4-[3-(dibutylamino)propyl]phenyl}carbonyl)-N-ethylindolizine-7-carboxamide are obtained in the form of a yellow foam.

Yield=50%.

### 11.3 2-Butyl-3-({4-[3-(dibutylamino)propyl]phenyl}carbonyl)-N-ethyl-N-(1H-tetrazol-5-ylmethyl)indolizine-7-carboxamide hydrochloride

A mixture of 1.32 g (2.02 mmol) of 2-butyl-N-{[1-(2-cyanoethyl)-1H-tetrazol-5-yl]methyl}-3-({4-[3-(dibutyl-amino)propyl]phenyl}carbonyl)-N-ethylindolizine-7-carboxamide in 5 ml of THF and 4 ml of a 1N aqueous NaOH solution is stirred for 18 h at AT. The reaction mixture is then neutralized with 4 ml of a 1N aqueous HCl solution, the THF is evaporated off, and then the resulting product is extracted with 2×50 ml of DCM. The organic phases are combined, washed successively with 50 ml of water and 50 ml of brine, dried over Na₂SO₄, filtered, treated with 2 ml of a 2N solution of hydrogen chloride in Et₂O, and then concentrated under reduced pressure. The residue obtained is triturated from ether, filtered, and then dried under vacuum. 1.19 g of 2-butyl-3-({4-[3-(dibutylamino)propyl]phenyl}carbonyl)-N-ethyl-N-(1H-tetrazol-5-ylmethyl)indolizine-7-carboxamide hydrochloride are thus obtained in the form of a hygroscopic foam.

Yield=92%.

LC/MS: M=C₃₅H₄₉N₇O₂=599; M+H=600; Tr=3.82 min (conditions B)

¹H NMR (ppm, d₆-DMSO, 400 MHz): 10.70-10.50 (bs, 1H); 9.40 (d, 1H); 7.80 (s, 1H); 7.55 (d, 2H); 7.40 (d, 2H); 7.00 (d, 1H); 6.65 (s, 1H); 4.45 (s, 2H); 3.55-3.40 (bs, 2H); 3.10-2.95 (bs, 6H); 2.80-2.70 (t, 2H); 2.30-2.20 (bs, 2H); 2.10-2.00 (bs, 2H); 1.70-1.60 (bs, 4H); 1.40-1.25 (bs, 6H); 1.15 (t, 3H); 1.05-0.95 (bs, 2H); 0.90 (t, 6H); 0.65 (t, 3H).

### EXAMPLE 12

### Compound No. 12: methyl N-[(2-butyl-3-{([4-(piperidin-4-yl)phenyl]carbonyl}indolizin-7-yl)carbonyl]-N-propan-2-ylglycinate hydrochloride

### 12.1 4-[1-(Trifluoroacetyl)piperidin-4-yl]benzoic acid

20.34 ml (146.16 mmol) of TFAA are added dropwise to a solution of 10.0 g (48.72 mmol) of 4-(piperidin-4-yl)benzoic acid in 490 ml of THF. After stirring for 1 h at AT, the reaction mixture is concentrated under reduced pressure, taken up with 500 ml of EtOAc, washed successively with 200 ml of water and 200 ml of brine, dried over MgSO₄, filtered, and then again concentrated under reduced pressure. The residue obtained is then triturated from pentane, filtered, and then dried under reduced pressure. 11.24 g of 4-[1-(trifluoro-acetyl)piperidin-4-yl]benzoic acid are thus obtained in the form of a whitish solid which is used as it is in the next step.

Yield=77%.

### 12.2 4-[1-(Trifluoroacetyl)piperidin-4-yl]benzoyl chloride

In a sealed tube, a mixture of 11.2 g (37.18 mmol) of 4-[1-(trifluoroacetyl)piperidin-4-yl]benzoic acid in 35 ml (483 mmol) of thionyl chloride is heated to 70° C. in the presence of a drop of DMF. After 5 h at 70° C., the reaction mixture is then concentrated under reduced pressure. 11.82 g of 4-[1-(trifluoroacetyl)piperidin-4-yl)benzoyl chloride are thus obtained in the form of a whitish solid which is used as it is in the next step.

Yield=100%.

### 12.3 Propan-2-yl 2-butyl-3-({4-[1-(trifluoroacetyl)piperidin-4-yl]phenyl}carbonyl)indolizine-7-carboxylate

A solution of 9.65 g (37.19 mmol) of 1-methylethyl 2-butylindolizine-7-carboxylate and of 4.8 g (37.19 mmol) of DIEA is added dropwise to a solution of 11.89 g (37.19 mmol) of 4-[1-(trifluoroacetyl)piperidin-4-yl]benzoyl chloride in 41 ml of THF and then the mixture is heated for 5 h at 85° C. At AT, the reaction mixture is concentrated under reduced pressure, taken up with 4.00 ml of EtOAc, washed successively with 200 ml of water and 200 ml of brine, dried over Na₂SO_{4f} filtered, and then again concentrated under reduced pressure. The residue obtained is purified by silica column chromatography, elution being carried out with a cyclohexane/EtOAc mixture of 0 to 30% with respect to EtOAc. After concentration under reduced pressure, 7.19 g of propan-2-yl 2-butyl-3-({4-[1-(trifluoroacetyl)piperidin-4-yl]phenyl}carbonyl)indolizine-7-carboxylate are obtained in the form of a whitish solid with a purity, determined by LC/MS, of 90%.

Yield=31%.

### 12.4 3-({4-[1-(tert-Butoxycarbonyl)piperidin-4-yl]phenyl}carbonyl)-2-butylindolizine-7-carboxylic acid

A mixture of 7.19 g (13.25 mmol) of propan-2-yl 2-butyl-3-({4-[1-(trifluoroacetyl)piperidin-4-yl]phenyl}carbonyl)indolizine-7-carboxylate and 4.58 g (33.13 mmol) of K₂CO₃ in 270 ml of MeOH is stirred for 2 h at AT. The reaction mixture is then concentrated under reduced pressure, taken up with 200 ml of water, and washed with 2×100 ml of DCM, and then the precipitate thus obtained in the aqueous phase is filtered off, washed with Et₂O and dried under reduced pressure. 1.6 g of a yellow solid are thus obtained, and placed in solution in 12 ml of a 2:1 0.5N aqueous NaOH/dioxane mixture to which 0.95 g (4.36 mmol) of Boc₂O is added. After stirring for 4 h at AT, the reaction mixture is cooled to 0° C., neutralized with 30 ml of a 0.2N aqueous HCl solution and then extracted with 2×150 ml of DCM. The organic phases are combined, washed with 50 ml of brine, dried over Na₂SO₄, filtered and then concentrated under reduced pressure. 2.29 g of 3-({4-[1-(tert-butoxycarbonyl)piperidin-4-yl]phenyl}carbonyl)-2-butylindolizine-7-carboxylic acid are thus obtained in the form of an amorphous powder which is used as it is in the next step.

Yield=34%.

### 12.5 tert-Butyl 4-[4-({2-butyl-7-[(2-methoxy-2-oxoeth-yl)(propan-2-yl)carbamoyl]indolizin-3-yl]carbonyl)phenyl}piperidine-1-carboxylate

With the exception of the salification step, the process is carried out in the same way as in example 2.2. Thus, starting from 2.29 g (4.54 mmol) of 3-({4-[1-tert-butoxycarbonyl)piperidin-4-yl]phenyl}carbonyl)-2-butylindolizine-7-carboxylic acid, 0.84 g (5.0 mmol) of methyl N-propan-2-ylglycinate hydrochloride, 1.76 g (13.63 mmol) of DIEA and 2.19 g (6.81 mmol) of TBTU in 22 ml of DCM, 1.99 g of tert-butyl 4-[4-({2-butyl-7-[(2-methoxy-2-oxoethyl)(propan-2-yl)carbamoyl]indolizin-3-yl]carbonyl)phenyl}piperidine-1-carboxylate are obtained in the form of a white foam.

Yield=67%.

### 12.6 Methyl N-[(2-butyl-3-{[4-(piperidin-4-yl)phenyl]carbonyl}indolizin-7-yl)carbonyl]-N-propan-2-ylglycinate hydrochloride

3 ml of a 4N solution of hydrogen chloride in dioxane are added to a solution of 1.99 g (3.22 mmol) of tert-butyl 4-[4-({2-butyl-7-[(2-methoxy-2-oxoethyl)(propan-2-yl)carbamoyl]indolizin-3-yl]carbonyl)phenyl}piperidine-1-carboxylate in 7 ml of DCM. After stirring for 8 h at AT, the reaction mixture is concentrated under reduced pressure, and the residue obtained is triturated from Et₂O, filtered, and then concentrated under reduced pressure. 1.4 g of methyl N-[(2-butyl-3-{[4-(piperidin-4-yl)phenyl]carbonyl}indolizin-7-yl)carbonyl]-N-propan-2-ylglycinate hydrochloride are thus obtained.

Yield=79%.

Mp (° C.): 123

LC/MS: M=C₃₁H₃₉N₃O₄=517; M+H=518; Tr=0.99 min (conditions B)

¹H NMR (ppm, d₆-DMSO, 400 MHz):

9.50 (d, 1H); 8.90-8.70 (bs, 2H); 7.65 (s, 1H); 7.60 (d, 2H); 7.40 (d, 1H); 6.90 (d, 1H); 6.65 (s, 1H); 4.10 (s, 2H); 4.10-3.95 (bs, 1H); 3.70 (s, 3H); 3.45-3.35 (bs, 2H); 3.10-2.90 (bs, 3H); 2.25-2.15 (bs, 2H); 2.05-1.80 (bs, 4H); 1.40-1.25 (bs, 2H); 1.25-1.10 (bs, 6H); 1.10-0.90 (bs, 2H); 0.65 (t, 3H).

### EXAMPLE 13

### Compound No. 13: 4-{[2-butyl-3-({4-[3-(dibutylamino)propyl]phenyl}carbonyl)indolizin-7-yl]carbonyl}piperazin-2-one hydrochloride

The process is carried out in the same way as in example 2.2. Thus, starting from 1.0 g (2.04 mmol) of 3-({4-[1-(tert-butoxycarbonyl)piperidin-4-yl]phenyl}carbonyl)-2-butylindolizine-7-carboxylic acid, 0.25 g (2.45 mmol) of piperazin-2-one, 0.66 g (5.1 mmol) of DIEA and 0.98 g (3.06 mmol) of TBTU in 10 ml of DCM, 0.88 g of 4-{[2-butyl-3-({4-[3-(dibutylamino)propyl]phenyl}carbonyl)indolizin-7-yl]carbonyl}piperazin-2-one hydrochloride is obtained in the form of a yellow solid, after purification on a silica column, elution being carried out with a MeOH/DCM gradient of 0 to 10% with respect to MeOH, followed by a salification step.

Yield=75%.

Mp (° C.): 162

LC/MS: M=C₃₅H₄₈N₄O₃=572; M+H=573; Tr=1.01 min (conditions B)

¹H NMR (ppm, d₆-DMSO, 400 MHz):

9.50-9.35 (bs, 2H); 8.15 (s, 1H); 7.80 (s, 1H); 7.60 (d, 2H); 7.45 (d, 2H); 7.00 (d, 1H); 6.70 (s, 1H); 4.10 (s, 2H); 3.80-3.60 (bs, 2H); 3.15-3.00 (bs, 6H); 2.80-2.70 (t, 2H); 2.30-2.20 (t, 2H); 2.05-1.90 (bs, 2H); 1.70-1.55 (bs, 4H); 1.50-1.25 (bs, 6H); 1.10-1.00 (bs, 2H); 0.95 (t, 6H); 0.65 (t, 3H).

### EXAMPLE 14

### Compound No. 14: methyl N-{[3-({4-[4-(cyclopentylamino)butyl]phenyl}carbonyl)-2-ethylindolizin-7-yl]carbonyl}-N-propan-2-ylglycinate hydrochloride

### 14.1 Propan-2-yl 3-{[4-(4-chlorobutyl)phenyl]carbonyl}-2-ethylindolizine-7-carboxylate

Except for the addition of DIEA, the process is carried out in the same way as in example 1.4. Thus, starting from 4.7 g (20.32 mmol) of propan-2-yl 2-ethylindolizine-7-carboxylate, 5.1 g (20.92 mmol) of 4-(4-chlorobutyl)benzoyl chloride and 2.63 g (20.32 mmol) of DIEA in 20 ml of anh. THF, 6.15 g of propan-2-yl 3-{[4-(4-chlorobutyl)phenyl]carbonyl}-2-ethylindolizine-7-carboxylate are obtained in the form of an orangey-colored gum.

Yield=71%.

### 14.2 Propan-2-yl 3-({4-[4-(cyclopentylamino)butyl]phenyl}carbonyl)-2-ethylindolizine-7-carboxylate

The process is carried out in the same way as in example 8.4. Thus, starting from 6.15 g (14.11 mmol) of propan-2-yl 3-{[4-(4-chlorobutyl)phenyl]carbonyl}-2-ethylindolizine-7-carboxylate, 4.92 g (57.75 mmol) of cyclopentylamine and 2.52 g (15.16 mmol) of KI in 35 ml of CH₃CN, 6.66 g of propan-2-yl 3-({4-[4-(cyclo-pentylamino)butyl]phenyl}carbonyl)-2-ethylindolizine-7-carboxylate are obtained in the form of a yellow powder.

Yield=97%.

### 14.3 Propan-2-yl 3-[(4-{4-[(tert-butoxycarbonyl) (cyclo-pentyl)amino]butyl}phenyl)carbonyl]-2-ethylindolizine-7-carboxylate

A mixture of 6.66 g (14.03 mmol) of propan-2-yl 3-({4-[4-(cyclopentylamino)butyl]phenyl}carbonyl)-2-ethylindolizine-7-carboxylate, 3.67 g (16.84 mmol) of Boc₂O and 1.42 g (14.03 mmol) of TEA in 60 ml of DCM is stirred for 18 h at AT. The mixture is then washed successively with 50 ml of water and 50 ml of brine, dried over Na₂SO₄, filtered, and then concentrated under reduced pressure. The residue obtained is purified by silica column chromatography, elution being carried out with an EtOAc/cyclohexane mixture of 0 to 30% with respect to EtOAc. After concentration under reduced pressure, 7.15 g of propan-2-yl 3-[(4-{4-[(tert-butoxycarbonyl) (cyclopentyl)amino]butyl}phenyl)carbonyl]-2-ethylindolizine-7-carboxylate are obtained in the form of an orangey-colored oil.

Yield=89%.

### 14.4 3-[((4-{4-[[(tert-Butoxycarbonyl)(cyclo-pentyl)amino]butyl}phenyl)carbonyl]-2-ethylindolizine-7-carboxylic acid

The process is carried out in the same way as in example 8.5. Thus, starting from 7.15 g (12.44 mol) of propan-2-yl 3-[(4-{4-[(tert-butoxycarbonyl)(cyclopentyl)amino]butyl}phenyl)carbonyl]-2-ethylindolizine-7-carboxylate, 6.016 g of 3-[(4-{4-[(tert-butoxycarbonyl)(cyclopentyl)amino]butyl}phenyl)carbonyl]-2-ethylindolizine-7-carboxylic acid are obtained in the form of a yellow powder.

Yield=91%.

### 14.5 Methyl N-({3-[(4-{4-[(tert-butoxycarbonyl)(cyclo-pentyl)amino]butyl}phenyl)carbonyl]-2-ethylindolizine-7-yl}carbonyl)-N-propan-2-ylglycinate

The process is carried out in the same way as in example 8.6. Thus, starting from 1.1 g (2.07 mol) of 3-[(4-{4-[(tert-butoxycarbonyl)(cyclopentyl)amino]butyl}phenyl)carbonyl]-2-ethylindolizine-7-carboxylic acid, 0.52 g (3.10 mmol) of methyl N-propan-2-ylglycinate hydrochloride, 0.80 g (6.20 mmol) of DIEA and 0.99 g (3.10 mmol) of TBTU, 1.3 g of methyl N-({3-[(4-{4-[(tert-butoxycarbonyl)(cyclopentyl)amino]butyl}phenyl)carbonyl]-2-ethylindolizine-7-yl}carbonyl)-N-propan-2-ylglycinate are obtained in the form of a yellow gum.

Yield=97%.

### 14.6 Methyl N-{[3-({4-[4-(cyclopentylamino)butyl]phenyl}carbonyl)-2-ethylindolizin-7-yl]carbonyl}-N-propan-2-ylglycinate hydrochloride

The process is carried out in the same way as in example 8.7. Thus, starting from 1.3 g (2.01 mmol) of methyl N-({3-[(4-{4-[(tert-butoxycarbonyl) (cyclo-pentyl)amino]butyl}phenyl)carbonyl]-2-ethylindolizine-7-yl}carbonyl)-N-propan-2-ylglycinate, 1.03 g of methyl N-{[3-({4-[4-(cyclopentylamino)butyl]phenyl}carbonyl)-2-ethylindolizin-7-yl]carbonyl}-N-propan-2-ylglycinate hydrochloride are obtained in the form of a yellow foam.

Yield=88%.

Mp (° C.): 154

LC/MS: M=C₃₃H₄₃N₃O₄=545; M+H=545; Tr=1.13 min (conditions B)

¹H NMR (ppm, d₆-DMSO, 400 MHz):

9.45-9.35 (bs, 1H); 8.75-8.60 (bs, 2H); 7.65 (s, 1H); 7.55 (d, 2H); 7.40 (d, 2H); 6.90 (d, 1H); 6.70 (s, 1H); 4.10 (s, 2H); 4.10-3.95 (bs, 1H); 3.70 (s, 3H); 3.50-3.35 (bs, 1H); 2.95-2.85 (t, 2H); 2.75-2.65 (t, 2H); 2.30-2.20 (bs, 2H); 2.00-1.85 (bs, 2H); 1.70-1.45 (bs, 10H); 1.15 (d, 6H); 1.05 (t, 3H).

### EXAMPLE 15

### Compound No. 15: methyl N-{[3-({4-[3-(1-aminocyclopentyl)propyl]phenyl}carbonyl)-2-ethylindolizine-7-yl]carbonyl}-N-propan-2-ylglycinate hydrochloride

### 15.1 Benzyl 1-(prop-2-yn-1-yl)cyclopentanecarboxylate

36.72 ml (58.75 mmol) of a 1.6 M solution of n-BuLi in n-hexane and 34.07 ml of HMPA are added dropwise, at −40° C. under argon, to a solution of 8.30 ml (58.75 mmol) of DIPA in 100 ml of anh. THF. After stirring for 15 min at −40° C., the reaction mixture is cooled to −78° C. and then a solution of 10.0 g (48.96 mmol) of benzyl cyclopentanecarboxylate is added dropwise. After stirring for 15 min at −78° C., 21.81 ml (195.82 mmol) of an 80% w/v solution of propargyl bromide in toluene are added dropwise. The reaction mixture is then left to return slowly to AT and, after 1 h, it is treated with 200 ml of a saturated aqueous solution of ammonium chloride and then extracted with 2×200 ml of EtOAc. The organic phases are combined, washed with 100 ml of brine, dried over MgSO₄, filtered, and then concentrated under reduced pressure. The residue obtained is purified by silica column chromatography, elution being carried out with an EtOAc/cyclohexane gradient of 0 to 30% with respect to EtOAc. After concentration under reduced pressure, 9.1 g of benzyl 1-(prop-2-yn-1-yl)cyclopentanecarboxylate are obtained in the form of an orangey-colored oil.

Yield=77%.

### 15.2 Propan-2-yl 2-ethyl-3-[(4-iodophenyl)carbonyl]indolizine-7-carboxylate

The process is carried out in the same way as in example 14.1. Thus, starting from 22.0 g (95.12 mmol) of propan-2-yl 2-ethylindolizine-7-carboxylate, 25.35 g (95.12 mmol) of 4-iodobenzyl chloride and 12.30 g (95.12 mmol) of DIEA in 100 ml of THF, 34.3 g of propan-2-yl 2-ethyl-3-[(4-iodophenyl)carbonyl]indolizine-7-carboxylate are obtained in the form of a yellow solid.

Yield=78%.

### 15.3 Propan-2-yl 3-{[4-(3-{1-[(benzyloxy)carbonyl]cyclopentyl}prop-1-yn-1-yl)phenyl]carbonyl}-2-ethylindolizine-7-carboxylate

A mixture of 12.60 g (27.31 mmol) of propan-2-yl 2-ethyl-3-[(4-iodophenyl)carbonyl]indolizine-7-carboxylate, 9.93 g (40.97 mmol) of benzyl 1-(prop-2-yn-1-yl)cyclopentanecarboxylate, 3.53 g (27.31 mmol) of DIEA and 0.31 g (1.64 mmol) of CuI in 54 ml of CH₃CN is stirred for 15 min at AT under argon, and then 0.77 g (1.09 mmol) of PdCl₂ (PPh₃) is added and the reaction mixture is then heated to 5 h at 50° C. At AT, the mixture is taken up with 300 ml of EtOAc, washed successively with 2×100 ml of water and 100 ml of brine, dried over MgSO₄, filtered, and then concentrated under reduced pressure. The residue obtained is purified by silica column chromatography, elution being carried out with an EtOAc/cyclohexane gradient of 0 to 10% with respect to EtOAc. After concentration under reduced pressure, 10.5 g of propan-2-yl 3-{[4-(3-{1-[(benzyloxy)carbonyl]cyclopentyl}prop-1-yn-1-yl)phenyl]carbonyl}-2-ethylindolizine-7-carboxylate are obtained in the form of an approximately 80% pure brown oil which is used as it is in the next step.

Yield=54% (corrected).

### 15.4 1-{3-[4-({2-Ethyl-7-[(propan-2-yloxy)carbonyl]indolizine-3-yl}carbonyl)phenyl]propyl}cyclopentane carboxylic acid

A mixture of 5.0 g (6.2 mmol corrected) of propan-2-yl 3-{[4-3-{1-[(benzyloxy)carbonyl]cyclopentyl}prop-1-yn-1-yl)phenyl]carbonyl}-2-ethylindolizine-7-carboxylate, 9.47 g (150 mmol) of ammonium formate and 0.6 g of 10% Pd—C in 50 ml of a 9:1 MeOH/dioxane mixture is heated under argon for 9 h at 90° C. The reaction mixture is then concentrated under reduced pressure, taken up with 300 ml of DCM, washed successively with 2×100 ml of water and 100 ml of brine, dried over MgSO₄, filtered, and then concentrated under reduced pressure. The residue obtained is purified by silica column chromatography, elution being carried out with an MeOH/DCM gradient of 0 to 5% with respect to MeOH. After concentration under reduced pressure, 3 g of 1-{3-[4-({2-ethyl-7-[(propan-2-yloxy)carbonyl]indolizin-3-yl}carbonyl)phenyl]propyl}cyclopentanecarboxylic acid are obtained in the form of a yellow solid.

Yield=70%.

### 15.5 Propan-2-yl 3-{[4-(3-{1-[(tert-butoxycarbonyl)amino]cyclopentyl}propyl)phenyl]carbonyl}-2-ethylindolizine-7-carboxylate

2.83 ml (3.61 mmol) of DPPA are added dropwise, at AT, to a solution of 5.36 g (10.95 mmol) of 1-{3-[4-({2-ethyl-7-[(propan-2-yloxy)carbonyl]indolizin-3-yl}carbonyl)phenyl]propyl}cyclopentanecarboxylic acid and 2.22 g (21.90 mmol) TEA in toluene. After stirring for 3 h at AT, the reaction mixture is taken up with 200 ml of EtOAc, washed successively with 2×50 ml of water and 50 ml of brine, dried over MgSO₄, filtered, and then concentrated under reduced pressure. In a sealed tube, a solution of the residue obtained and 0.1 g (1 mmol) of CuCl in 50 ml of anh. t-BuOH is heated for 18 h at 115° C. The reaction mixture is concentrated under reduced pressure and then purified by silica column chromatography, elution being carried out with an EtOAc/cyclohexane gradient of 0 to 15% with respect to EtOAc. After concentration under reduced pressure, 4.0 g of propan-2-yl 3-{[4-(3-{1-[(tert-butoxycarbonyl)amino]cyclopentyl}propyl)phenyl]carbonyl}-2-ethylindolizine-7-carboxylate are obtained in the form of a yellow oil.

Yield=65%.

### 15.6 3-{[4-(3-{1-[(tert-Butoxycarbonyl)amino]cyclopentyl}propyl)phenyl]carbonyl}-2-ethylindolizine-7-carboxylic acid

The process is carried out in the same way as in example 8.5. Thus, starting from 0.50 g (0.9 mmol) of propan-2-yl 3-{[4-(3-{1-[(tert-butoxycarbonyl)amino]-cyclopentyl}propyl)phenyl]carbonyl}-2-ethylindolizine-7-carboxylate, 0.48 g of 3-{[4-(3-{1-[(tert-butoxycarbonyl)amino]cyclopentyl}propyl)phenyl]carbonyl}-2-ethylindolizine-7-carboxylic acid is obtained in the form of a yellow foam.

Yield=100%.

### 15.7 Methyl N-[(3-{[4-(3-{1-[(tert-butoxycarbonyl)amino]cyclopentyl}propyl)phenyl]carbonyl}-2-ethylindolizine-7-yl)carbonyl]-N-propan-2-ylglycinate

The process is carried out in the same way as in example 8.6. Thus, starting from 0.48 g (0.94 mmol) of 3-{[4-(3-{1-[(tert-butoxycarbonyl)amino]cyclo-pentyl}propyl)phenyl]carbonyl}-2-ethylindolizine-7-yl)carboxylic acid, methyl N-propan-2-ylglycinate hydrochloride, 0.36 g (2.81 mmol) of DIEA and 0.45 g (1.40 mmol) of TBTU, 0.43 g of methyl N-[(3-{[4-(3-{1-[(tert-butoxycarbonyl)amino]cyclo-pentyl}propyl)phenyl]carbonyl}-2-ethylindolizine-7-yl)carbonyl]-N-propan-2-ylglycinate is obtained in the form of a yellow foam.

Yield=74%.

### 15.8 Methyl N-{[3-({4-[3-(1-aminocyclopentyl)propyl]phenyl}carbonyl)-2-ethylindolizin-7-yl]carbonyl}-N-propan-2-ylglcinate hydrochloride

The process is carried out in the same way as in example 8.7. Thus, starting from 0.435 g (0.69 mol) of methyl N-[(3-{[4-(3-{1-[(tert-butoxycarbonyl)amino]cyclopentyl}propyl)phenyl]carbonyl}-2-ethylindolizine-7-yl)carbonyl]-N-propan-2-ylglycinate, 0.272 g of methyl N-{[3-({4-[3-(1-aminocyclopentyl)propyl]phenyl}carbonyl)-2-ethylindolizin-7-yl]carbonyl}-N-propan-2-ylglycinate hydrochloride is obtained in the form of a yellow powder.

Yield=69%.

Mp (° C.): 176

LC/MS: M=C₃₂H₄₁N₃O₄=531; M+H=532; Tr=1.13 min (conditions B)

¹H NMR (ppm, d₆-DMSO, 400 MHz):

9.40 (d, 1H); 7.95-7.80 (bs, 3H); 7.65 (s, 1H); 7.55 (d, 2H); 7.40 (d, 2H); 6.90 (d, 1H); 6.70 (s, 1H); 4.10 (s, 2H); 4.05-3.95 (bs, 1H); 3.75 (s, 3H); 2.80-2.70 (t, 2H); 2.30-2.20 (bs, 2H); 1.80-1.50 (bs, 12H); 1.15 (d, 6H); 1.00 (t, 3H).

### EXAMPLE 16

### Compound No. 16: methyl N-({2-ethyl-3-[(4-{(3-[1-(methylamino)cyclopentyl]propyl}phenyl)carbonyl]indolizin-7-yl}carbonyl)-N-propan-2-ylglycinate hydrochloride

### 16.1 3-{[4-(3-{1-[(tert-Butoxycarbonyl) (methyl)amino]cyclopentyl}propyl)phenyl]carbonyl}-2-ethylindolizine-7-carboxylic acid

140 mg (3.43 mmol) of NaH at 60% in oil are added in small amounts, at AT under argon, to a solution of 970 mg (1.73 mmol) of propan-2-yl 3-{[4-(3-{1-[(tert-butoxycarbonyl)amino]cyclopentyl}propyl)phenyl]carbonyl}-2-ethylindolizine-7-carboxylate in 5 ml of anh. DMF. After 15 min, 220 μl (3.43 mmol) of iodomethane are added dropwise at AT and the stirring is continued for 18 h. The reaction mixture is then treated with 50 ml of a saturated aqueous NH₄Cl solution, and then extracted with 2×100 ml of ether. The organic phases are combined, washed successively with 2×50 ml of water and 50 ml of brine, dried over Na₂SO₄, filtered, and then concentrated under reduced pressure. The residue obtained is taken up with 10 ml of dioxane and then 4 ml of a 1N aqueous NaOH solution are added dropwise at AT. After stirring for 18 h, the reaction mixture is cooled to 0° and then neutralized with 4 ml of a 1N aqueous HCl solution and extracted with 2×100 ml of EtOAc. The organic phases are combined, washed successively with 2×50 ml of water and 50 ml of brine, dried over Na₂SO₄, filtered, and then concentrated under reduced pressure. The residue obtained is chromatographed on a silica gel column, elution being carried out with a DCM/MeOH gradient of 0 to 20% with respect to MeOH. After concentration under reduced pressure, 733 mg of 3-{[4-(3-{1-[(tert-butoxycarbonyl)(methyl)amino]cyclopentyl}propyl)phenyl]carbonyl}-2-ethylindolizine-7-carboxylic acid are obtained in the form of an orangey-colored solid which is used as it is in the next step.

Yield=77%.

### 16.2 Methyl N-[(3-{[4-(3-{1-[(tert-butoxycarbonyl)(methyl)amino]cyclopentyl}propyl)phenyl]carbonyl}-2-ethylindolizin-7-yl)carbonyl]-N-propan-2-ylglycinate

With the exception of the final salification step, the process is carried out in the same way as in example 2.2. Thus, starting from 1.1 g (2.08 mmol) of 3-{[4-(3-{1-[(tert-butoxycarbonyl)(methyl)amino]cyclo-pentyl}propyl)phenyl]carbonyl}-2-ethylindolizine-7-carboxylic acid and 0.7 g (4.17 mmol) of methyl N-propan-2-ylglycinate hydrochloride, 1.07 g of methyl N-[(3-{[4-(3-{1-[(tert-butoxycarbonyl)(methyl)amino]cyclopentyl}propyl)phenyl]carbonyl}-2-ethylindolizin-7-yl)carbonyl]-N-propan-2-ylglycinate are obtained in the form of a yellow foam, after chromatography on a silica gel column, elution being carried out with a cyclohexane/EtOAc gradient of 0 to 50% with respect to EtOAc.

Yield=80%.

### 16.3 Methyl N-({2-ethyl-3-[(4-{3-[1-(methylamino)cyclo-pentyl]propyl}phenyl)carbonyl]indolizin-7-yl}carbonyl)-N-propan-2-ylglycinate hydrochloride

The process is carried out in the same way as in example 11.3.

Thus, starting from 1.68 g (2.60 mmol) of methyl N-[(3-{[4-(3-{1-[(tert-butoxycarbonyl)(methyl)amino]cyclo-pentyl}propyl)phenyl]carbonyl}-2-ethylindolizin-7-yl)carbonyl]-N-propan-2-ylglycinate, of methyl N-({2-ethyl-3-[(4-[3-[1-(methylamino)cyclopentyl]propyl}phenyl)carbonyl]indolizin-7-yl]carbonyl)-N-propan-2-ylglycinate hydrochloride is obtained in the form of a yellow powder, after chromatography on an RP18 reverse phase column, elution being carried out with a CH₃CN/H₂O (0.01N HCl) gradient of 0 to 30% with respect to CH₃CN.

Yield=45%.

Mp (° C.): 149.5

LC/MS: M=C₃₃H₄₃N₃O₄=545; M+H=546; Tr=1.13 min (conditions B)

¹H NMR (ppm, d₈-DMSO, 400 MHz, T=60° C.)

9.40 (d, 1H); 8.70-8.60 (bs, 2H); 7.65 (s, 1H); 7.55 (d, 2H); 7.40 (d, 2H); 6.85 (d, 1H); 6.70 (s, 1H); 4.15-4.05 (bs, 3H); 3.70 (s, 3H); 2.75 (t, 2H); 2.50-2.40 (bs, 3H); 2.30-2.20 (bs, 2H); 1.90-1.50 (bs, 12H); 1.15 (d, 6H); 1.00 (t, 3H).

### EXAMPLE 17

### Compound No. 17: 3-({4-[3-(tert-butyl-amino)propyl]phenyl}carbonyl)-N,2-diethyl-N-[(2-methyl-2H-tetrazol-5-yl)methyl]indolizine-7-carboxamide hydrochloride

### 17.1 Propan-2-yl 3-({4-[3-(tert-butylamino)propyl]phenyl}carbonyl)-2-ethylindolizine-7-carboxylate

In a sealed tube, a mixture of 2.35 g (5.7 mmol) of propan-2-yl 3-{[4-(4-chlorobutyl)phenyl]carbonyl}-2-ethylindolizine-7-carboxylate, 2.4 ml (22.8 mmol) of tert-butylamine and 0.99 g (5.9 mmol) of KI in 12 ml of CH₃CN is heated for 48 h at 105° C. The reaction mixture is taken up with 100 ml of EtOAc, washed successively with 2×30 ml of water and 30 ml of brine, dried over Na₂SO₄, filtered, and then concentrated under reduced pressure. The residue obtained is solidified from ether, filtered and washed with ether. 3.53 g of propan-2-yl 3-({4-[3-(tert-butylamino)propyl]phenyl}carbonyl)-2-ethylindolizine-7-carboxylate are thus obtained in the form of a yellow powder which is used as it is in the next step.

Yield=70%.

### 17.2 3-({4-[3-(tert-Butylamino)propyl]phenyl}carbonyl)-2-ethylindolizine-7-carboxylic acid

16 ml of a 1N aqueous NaOH solution are added, dropwise at AT, to a solution of 3.53 g (7.9 mmol) of propan-2-yl 3-({4-[3-(tert-butylamino)propyl]phenyl}carbonyl)-2-ethylindolizine-7-carboxylate in 16 ml of a 2:1:1 dioxane/MeOH/THF mixture and the stirring is continued for 18 h. The mixture is cooled to 0° C. and then 16 ml of a 1N aqueous HCl solution are added dropwise. The resulting precipitate is then filtered off, washed with water and then dried under reduced pressure. 3.1 g of 3-({4-[3-(tert-butylamino)propyl]phenyl}carbonyl)-2-ethylindolizine-7-carboxylic acid are thus obtained in the form of a yellow powder which is used as it is in the next step.

Yield=99%.

### 17.3 3-({4-[3-(tert-Butylamino)propyl]phenyl}carbonyl)-N,2-diethyl-N-[(2-methyl-2H-tetrazol-5-yl)methyl]indolizine-7-carboxamide hydrochloride

The process is carried out in the same way as in example 2.2. Thus, starting from 0.8 g (1.97 mmol) of 3-({4-[3-(tert-butylamino)propyl]phenyl}carbonyl)-2-ethylindolizine-7-carboxylic acid and 0.36 g (2.56 mmol) of N-[(2-methyl-2H-tetrazol-5-yl)methyl]ethaneamine hydrochloride, 0.9 g are obtained, after silica column chromatography, elution being carried out with a DCM/MeOH gradient of 0 to 10% with respect to MeOH, and taken up with 5 ml of DCM, and then the resulting product is cooled to 0° C., 1.70 ml of a 2N solution of hydrogen chloride in ether are added and the resulting mixture is left to return slowly to AT. The precipitate obtained is filtered off, washed with ether, and then dried under reduced pressure. 0.88 g of 3-({4-[3-(tert-butylamino)propyl]phenyl}carbonyl)-N,2-diethyl-N-[(2-methyl-2H-tetrazol-5-yl)methyl]indolizine-7-carboxamide hydrochloride is thus obtained in the form of a white powder.

Yield=84%.

Mp (° C.): 175

LC/MS: M=C₃₀H₃₉N₇O₂=529; M+H=530; Tr=1.04 min (conditions B)

¹H NMR (ppm, d₆-DMSO, 400 MHz):

9.50 (d, 1H); 9.00-8.80 (bs, 2H); 7.80-7.65 (bs, 1H); 7.55 (d, 2H); 7.40 (d, 2H); 6.95-6.85 (bs, 1H); 6.70-6.60 (bs, 1H); 4.95-4.80 (bs, 2H); 4.35 (s, 3H); 3.50-3.35 (bs, 2H); 2.95-2.70 (bs, 4H); 2.30-2.15 (bs, 2H); 2.10-1.95 (t, 2H); 1.25 (s, 9H); 1.20-0.95 (bs, 6H).

### EXAMPLE 18

### Compound No. 18: 3-({4-[3-(tert-butyl-amino)-3-methylbutyl]phenyl}carbonyl)-N,2-diethyl-N-[(2-methyl-2H-tetrazol-5-yl)methyl]indolizine-7-carboxamide hydrochloride

### 18.1 Propan-2-yl 3-({4-[3-(tert-butylamino)-3-methylbut-1-yn-1-yl]phenyl}carbonyl)-2-ethylindolizine-7-carboxylate

The process is carried out in the same way as in example 15.3. Thus, starting from 2.2 g (4.77 mmol) of propan-2-yl 2-ethyl-3-[(4-iodophenyl)carbonyl]indolizine-7-carboxylate and 1.27 ml (7.15 mmol) of N-tert-butyl-2-methylbut-3-yn-2-amine, 2.5 g of 3-({4-[3-(tert-butylamino)-3-methylbut-1-yn-1-yl]phenyl}carbonyl)-2-ethylindolizine-7-carboxylate are obtained in the form of an oil, after silica column chromatography, elution being carried out with a cyclohexane/EtOAc gradient of 0 to 40% with respect to EtOAc.

Yield=100%.

### 18.2 Propan-2-yl 3-({4-[3-(tert-butylamino)-3-methylbutyl]phenyl}carbonyl)-2-ethylindolizine-7-carboxylate

A mixture of 2.5 g (5.29 mmol) of propan-2-yl 3-({4-[3-(tert-butylamino)-3-methylbut-1-yn-1-yl]phenyl}carbonyl)-2-ethylindolizine-7-carboxylate and 1.7 g of 10% Pd—C in 20 ml of a 1:1 EtOAc/EtOH mixture is stirred for 1 h under 3 bar of hydrogen. The reaction medium is subsequently filtered and then concentrated under reduced pressure. The residue obtained is chromatographed on a silica column, elution being carried out with a cyclohexane/EtOAc gradient of 0 to 40% with respect to EtOAc. After concentration under reduced pressure, 1.17 g of propan-2-yl 3-({4-[3-(tert-butylamino)-3-methylbutyl]phenyl}carbonyl)-2-ethylindolizine-7-carboxylate are obtained in the form of an orangey-colored gum.

Yield=46%.

### 18.3 3-({4-[3-(tert-Butylamino)-3-methylbutyl]phenyl}carbonyl)-N,2-diethyl-N-[(2-methyl-2H-tetrazol-5-yl)methyl]indolizine-7-carboxamide hydrochloride

By applying a saponification-peptide coupling sequence as described in examples 17.2 and 17.4 respectively, and starting from 0.79 g (1.81 mmol) of propan-2-yl 3-({4-[3-(tert-butylamino)-3-methylbutyl]phenyl}carbonyl)-2-ethylindolizine-7-carboxylate, and after a final trituration in ether, 0.52 g of 3-({4-[3-(tert-butylamino)-3-methylbutyl]phenyl}carbonyl)-N,2-diethyl-N-[(2-methyl-2H-tetrazol-5-yl)methyl]indolizine-7-carboxamide hydrochloride is obtained in the form of a green powder.

Yield=48%.

Mp (° C.): 122

LC/MS: M=C₃₂H₄₃N₇O₂=557; M+H=558; Tr=1.05 min (conditions B)

¹H NMR (ppm, d₆-DMSO, 400 MHz):

9.45-9.30 (bs, 1H); 8.30-8.10 (bs, 2H); 7.80-7.65 (bs, 1H); 7.55 (d, 2H); 7.40 (d, 2H); 7.00-6.85 (bs, 1H); 7.65 (s, 1H); 5.00-4.80 (bs, 2H); 4.35 (s, 3H); 3.60-3.30 (bs, 2H); 2.80-2.70 (bs, 2H); 2.25-2.15 (bs, 2H); 2.15-2.05 (bs, 2H); 1.55-1.40 (bs, 15H); 1.20-1.05 (bs, 3H); 1.05-0.95 (bs, 3H).

### EXAMPLE 19

### Compound No. 19: methyl N-{[3-({4-[3-(cyclopentylamino)-3-methylbutyl]phenyl}carbonyl)-2-ethylindolizin-7-yl]carbonyl}-N-propan-2-ylglycinate hydrochloride

### 19.1 Propan-2-yl 3-{[4-(3-amino-3-methylbut-1-yn-1-yl)phenyl]carbonyl}-2-ethylindolizine-7-carboxylate

The process is carried out in the same way as in example 15.3. Thus, starting from 6.0 g (13.01 mmol) of propan-2-yl 2-ethyl-3-[(4-iodophenyl)carbonyl]-indolizine-7-carboxylate and 1.68 ml (15.61 mmol) of 2-methylbut-3-yn-2-amine, 5.15 g of propan-2-yl 3-{[4-(3-amino-3-methylbut-1-yn-1-yl)phenyl]carbonyl}-2-ethylindolizine-7-carboxylate are obtained in the form of a yellow solid, after silica column chromatography, elution being carried out with a DCM/MeOH gradient of 0 to 10% with respect to MeOH.

Yield=95%.

### 19.2 Propan-2-yl 3-({4-[3-(cyclopentylamino)-3-methylbut-1-yn-1-yl]phenyl}carbonyl)-2-ethylindolizine-7-carboxylate

A solution of 5.15 g (12.36 mmol) of propan-2-yl 3-{[4-(3-amino-3-methylbut-1-yn-1-yl)phenyl]carbonyl}-2-ethylindolizine-7-carboxylate and 2.19 ml (24.7 mmol) of cyclopentanone in 25 ml of DCE is stirred for 2 h at AT and then 0.71 ml (12.36 mmol) of AcOH and 3.14 g (14.84 mmol) of NaBH(OAc)₃ are successively added. After stirring for 24 h at AT, the reaction mixture is treated with 20 ml of a saturated aqueous NaHCO₃ solution and then extracted with 2×50 ml of EtOAc. The organic phases are combined, dried over Na₂SO₄, filtered, and then concentrated under reduced pressure. The residue obtained is chromatographed on a silica column, elution being carried out with a DCM/MeOH gradient of 0 to 5% with respect to MeOH. After concentration under reduced pressure, 5.99 g of propan-2-yl 3-({4-[3-(cyclopentylamino)-3-methylbut-1-yn-1-yl]phenyl}carbonyl)-2-ethylindolizine-7-carboxylate are obtained in the form of a brown-green solid which is used as it is in the next step.

Yield=100%.

### 19.3 Methyl N-{[3-({4-[3-(cyclopentylamino)-3-methylbutyl]phenyl}carbonyl)-2-ethylindolizine-7-yl]carbonyl}-N-propan-2-ylglycinate hydrochloride

By applying a reduction-saponification-peptide coupling sequence, as described in examples 18.3, 17.2 and 8.6 respectively, and starting from 1.60 g (3.58 mmol) of propan-2-yl 3-({4-[3-(cyclopentylamino)-3-methylbut-1-yn-1-yl]phenyl}carbonyl)-2-ethylindolizine-7-carboxylate, 1.58 g of methyl N-{[3-({4-[3-(cyclopentylamino)-3-methylbutyl]phenyl}carbonyl)-2-ethylindolizine-7-yl]carbonyl}-N-propan-2-ylglycinate hydrochloride are obtained in the form of a yellow powder.

Yield=79%.

Mp (° C.): 245

LC/MS: M=C₃₄H₄₅N₃O₄=559; M+H=560; Tr=1.18 min (conditions B)

¹H NMR (ppm, d₅-DMSO, 400 MHz):

9.50-9.35 (bs, 1H); 8.60-8.40 (bs, 2H); 7.65 (s, 1H); 7.60 (d, 2H); 7.40 (d, 2H); 6.90 (d, 1H); 6.70 (s, 1H); 4.15 (s, 2H); 4.10-4.00 (bs, 1H); 3.80-3.60 (bs, 4H); 2.80-2.70 (bs, 2H); 2.30-2.20 (bs, 2H); 2.10-1.90 (bs, 4H); 1.80-1.70 (bs, 4H); 1.70-1.50 (bs, 2H); 1.40 (s, 6H); 1.10 (d, 6H); 1.00 (t, 3H).

### EXAMPLE 20

### Compound No. 20: (R,S)—N,2-diethyl-3-({4-[3-(ethylamino)-4-methylpentyl]phenyl}carbonyl)-N-[(2-methyl-2H-tetrazol-5-yl)methyl]indolizine-7-carboxamide hydrochloride

20.1 Propan-2-yl 3-{[4-(chloromethyl)phenyl]carbonyl}-2-ethylindolizine-7-carboxylate

10.61 g (56.14 mmol) of 4-(chloromethyl)benzoyl chloride are added to a solution of 8.66 g (37.43 mmol) of propan-2-yl 2-ethylindolizine-7-carboxylate, 8.69 ml (74.86 mmol) of lutidine and 0.61 ml (7.49 mmol) of pyridine in 75 ml of chlorobenzene and then the mixture is refluxed for 2 h. The reaction mixture is then taken up with 300 ml of EtOAc, washed successively with 2×150 ml of water and 150 ml of brine, dried over Na₂SO₄, filtered, and then concentrated under reduced pressure. The residue obtained is chromatographed on a silica gel column, elution being carried out with a cyclohexane/EtOAc gradient of 0 to 10% with respect to EtOAc. After concentration under reduced pressure, 10.44 g of propan-2-yl 3-{[4-(chloromethyl)phenyl]carbonyl}-2-ethylindolizine-7-carboxylate are obtained in the form of a yellow solid.

Yield=72%.

### 20.2 Propan-2-yl 3-({4-[(diethoxyphosphoryl)methyl]-phenyl}carbonyl)-2-ethylindolizine-7-carboxylate

A mixture of 15.0 g (39.08 mmol) of propan-2-yl 3-{[4-(chloromethyl)phenyl]carbonyl}-2-ethylindolizine-7-carboxylate and 26.80 ml of triethyl phosphite is refluxed for 3 h. The excess triethyl phosphite is evaporated off under reduced pressure. The residue obtained is taken up with 500 ml of EtOAc, washed successively with 2×200 ml of water and 100 ml of brine, dried over Na₂SO₄, filtered, and then concentrated under reduced pressure. The residue obtained is chromatographed on a silica column, elution being carried out with a cyclohexane/EtOAc gradient of 0 to 100%. After concentration under reduced pressure, 12.8 g of propan-2-yl 3-({4-[(diethoxyphosphoryl)methyl]phenyl}carbonyl)-2-ethylindolizine-7-carboxylate are obtained in the form of a brown oil which is used as it is in the next step.

Yield=68%.

### 20.3 N²-(tert-butoxycarbonyl)-N²-ethyl-N-methoxy-N-methyl-D,L-valinamide

1.66 g (41.37 mmol) of NaH at 60% in oil are added in small amounts, at 0° C., under argon, to a solution of 7.18 g (27.58 mmol) of N²-(tert-butoxycarbonyl)-N-methoxy-N-methyl-D,L-valinamide in 92 ml of anh. NMP. After 15 min at 0° C., 4.41 ml (55.16 mmol) of iodoethane are added dropwise, then the reaction mixture is allowed to return slowly to AT and the stirring is continued for 18 h. The residue obtained is chromatographed on a silica column, elution being carried out with a cyclohexane/EtOAc gradient of 0 to 40% with respect to EtOAc. After concentration under reduced pressure, 6.25 g of N²-(tert-butoxycarbonyl)-W-ethyl-N-methoxy-N-methyl-D,L-valinamide are obtained in the form of a colorless oil.

Yield=79%.

### 20.4 tert-Butyl (R,S)-ethyl[3-methyl-1-oxobutan-2-yl]carbamate

3.64 ml (3.64 mmol) of a 1N solution of LiAlH₄ in THF are added dropwise, under argon, at −78° C., to a solution of 1.0 g (3.47 mmol) of N²-(tert-butoxycarbonyl)-N²-ethyl-N-methoxy-N-methyl-D,L-valinamide in 11 ml of anh. THF. After stirring for 10 min, the reaction mixture is stirred at 0° C. for 10 min, diluted with 40 ml of ether, and treated successively with ˜2 g of ammonium chloride added in small amounts and water added dropwise until two liquid phases are obtained. The supernatant is then removed, washed successively with 20 ml of a 1N aqueous HCl solution and 20 ml of brine, dried over Na₂SO₄, filtered, and then concentrated under reduced pressure. After concentration under reduced pressure, 1.17 g of Cert-butyl (R,S)-ethyl[3-methyl-1-oxobutan-2-yl]carbamate are obtained in the form of a colorless oil which is used as it is in the next step.

Yield=100%.

### 20.5 Propan-2-yl (R,S)-3-[(4-{(1E)-3-[(tert-butoxycarbonyl)(ethyl)amino]-4-methylpent-1-en-1-yl}phenyl)carbonyl]-2-ethylindolizine-7-carboxylate

0.15 g (3.64 mmol) of NaH at 50% in oil is added in small amounts, under argon, at 0° C., to a solution of 1.7 g (3.50 mmol) of propan-2-yl 3-({4-[(diethoxyphosphoryl)methyl]phenyl}carbonyl)-2-ethylindolizine-7-carboxylate in 10 ml of anh. THF. After 30 min at 0° C., the reaction mixture is cooled to −78° C. and then a solution of 1.17 g (3.47 mmol) of tert-butyl (R,S)-ethyl[3-methyl-1-oxobutan-2-yl]carbamate in 5 ml of anh. THF is added dropwise. The reaction mixture is allowed to return slowly to AT and the stirring is continued for 18 h. The reaction mixture is then again cooled to 0° C., treated with 30 ml of a saturated aqueous NH₄Cl solution and then extracted with 3×70 ml of EtOAc. The organic phases are combined, washed successively with 50 ml of a 1N aqueous HCl solution, 50 ml of water and 50 ml of brine, dried over Na₂SO₄, filtered, and then concentrated under reduced pressure. The residue obtained is chromatographed on a silica column, elution being carried out with a cyclohexane/EtOAc gradient of 0 to 30% with respect to EtOAc and then with a DCM/MeOH gradient of 0 to 10% with respect to MeOH. After concentration under reduced pressure, 1.32 g of propan-2-yl (R,S)-3-[(4-{(1E)-3-[(tert-butoxycarbonyl) (ethyl)-amino]-4-methylpent-1-en-1-yl]phenyl)carbonyl}-2-ethylindolizine-7-carboxylate are obtained in the form of an orangey-colored gum.

Yield=68%.

20.6 (R,S)-3-[(4-{(1E)-3-[(tert-Butoxycarbonyl) (ethyl)-amino]-4-methylpent-1-en-1-yl]phenyl)carbonyl}-2-ethylindolizine-7-carboxylic acid

7.1 ml (7.1 mmol) of a 1N aqueous NaOH solution are added dropwise, at 0° C., to a solution of 1.98 g (3.53 mmol) of propan-2-yl (R,S)-3-[(4-{(1E)-3-[(tert-butoxycarbonyl) (ethyl)amino]-4-methylpent-1-en-1-yl]phenyl)carbonyl}-2-ethylindolizine-7-carboxylate in 22 ml of a 10:1 THF/MeOH mixture and then the reaction mixture is allowed to return slowly to AT. After stirring for 18 h, the reaction mixture is cooled to 0° C., neutralized with 7.1 ml of a 1N HCl solution and extracted with 3×70 ml of a 95:5 DCM/iPrOH mixture. The organic phases are combined, washed with 50 ml of brine, dried over Na₂SO₄, filtered, and then concentrated under reduced pressure. 2.19 g of (R,S)-3-[(4-{(1E)-3-[(tert-butoxycarbonyl)(ethyl)amino]-4-methylpent-1-en-1-yl]phenyl)carbonyl}-2-ethylindolizine-7-carboxylic acid are thus obtained in the form of a yellow powder which is used as it is in the next step.

Yield=94%.

### 20.7 tert-Butyl (R,S)-ethyl[(1E)-1-{4-[(2-ethyl-7-{ethyl[(2-methyl-2H-tetrazol-5-yl)methyl]carbamoyl}-indolizin-3-yl)carbonyl]phenyl}-4-methylpent-1-en-3-yl]carbamate

The process is carried out in the same way as in example 2.2. Thus, starting from 1.25 g (2.41 mmol) of (R,S)-3-[(4-{(1E)-3-[(tert-butoxycarbonyl)(ethyl)-amino]-4-methylpent-1-en-1-yl}phenyl)carbonyl)-2-ethylindolizine-7-carboxylic acid and 0.47 g (2.65 mmol) of N-[(2-methyl-2H-tetrazol-5-yl)methyl]ethanamine hydrochloride, 1.4 g of tert-butyl (R,S)-ethyl[(1E)-1-{4-[(2-ethyl-7-{ethyl[(2-methyl-2H-tetrazol-5-yl)methyl]carbamoyl}indolizin-3-yl)carbonyl]phenyl}-4-methylpent-1-en-3-yl]carbamate are obtained in the form of a yellow foam, after silica column chromatography, elution being carried out with a cyclohexane/EtOAc gradient of 0 to 100% of EtOAc.

Yield=91%.

### 20.8 tert-Butyl (R,S)-ethyl[1-{4-[(2-ethyl-7-{ethyl[(2-methyl-2H-tetrazol-5-yl)methyl]carbamoyl}indolizine-3-yl)carbonyl]phenyl}-4-methylpentan-3-yl]carbamate

A mixture of 1.4 g (2.19 mmol) of tert-butyl (R,S)-ethyl[(1E)-1-{4-[(2-ethyl-7-{ethyl[(2-methyl-2H-tetrazol-5-yl)methyl]carbamoyl}indolizin-3-yl)carbonyl]phenyl}-4-methylpent-1-en-3-yl]carbamate and 0.14 g of Pd—C at 10% in 30 ml of MeOH is stirred for 3 h under 5 bar of hydrogen. The reaction mixture is subsequently filtered and then concentrated under reduced pressure. The residue obtained is chromatographed on a silica column, elution being carried out with a DCM/MeOH gradient of 0 to 10% with respect to MeOH. After concentration under reduced pressure, 1.12 g of tert-butyl (R,S)-ethyl[1-{4-[(2-ethyl-7-{ethyl[(2-methyl-2H-tetrazol-5-yl)methyl]carbamoyl}indolizine-3-yl)carbonyl]phenyl}-4-methylpentan-3-yl]carbamate are obtained in the form of a yellow foam.

Yield=81%.

### 20.9 (R,S)—N,2-Diethyl-3-({4-[3-(ethylamino)-4-methylpentyl]phenyl}carbonyl)-N-[(2-methyl-2H-tetrazol-5-yl)methyl]indolizine-7-carboxamide hydrochloride

The process is carried out in the same way as in example 8.7. Thus, starting from 1.12 g (1.78 mmol) of Cert-butyl (R,S)-ethyl[1-{4-[(2-ethyl-7-{ethyl[(2-methyl-2H-tetrazol-5-yl)methyl]carbamoyl}indolizine-3-yl)carbonyl]phenyl}-4-methylpentan-3-yl]carbamate, 0.77 g of (R,S)—N,2-Diethyl-3-({4-[3-(ethylamino)-4-methylpentyl]phenyl}carbonyl)-N-[(2-methyl-2H-tetrazol-5-yl)methyl]indolizine-7-carboxamide hydrochloride is obtained in the form of a greenish powder.

Yield=93%.

Mp (° C.): 100

[α]_{D}²⁰=+4.2 (c=0.19; MeOH)

LC/MS: M=C₃₁H₄₁N₇O₂=543; M+H=544; Tr=1.05 min (conditions B)

¹H NMR (ppm, d₆-DMSO, 400 MHz):

9.45-9.35 (bs, 1H); 8.80-8.60 (bs, 1H); 8.30-8.10 (bs, 1H); 7.80-7.70 (bs, 1H); 7.55 (d, 2H); 7.45 (d, 2H); 7.00-6.90 (bs, 1H); 6.75-6.85 (bs, 1H); 5.00-4.80 (bs, 2H); 4.40 (s, 3H); 3.50-3.35 (bs, 2H); 3.15-2.75 (bs, 5H); 2.30-2.15 (bs, 2H); 2.15-2.00 (bs, 1H); 2.00-1.80 (bs, 2H); 1.35-0.80 (bs, 15H).

### EXAMPLE 21

### Compound No. 81: methyl N-{([2-butyl-3-({4-[(3R)-piperidin-3-yloxy]phenyl}carbonyl)indolizin-7-yl]carbonyl}-N-propan-2-ylglycinate hydrochloride

### 21.1 Propan-2-yl 2-butyl-3-[(4-iodophenyl)carbonyl]-indolizine-7-carboxylate

The process is carried out in the same way as in example 14.1. Thus, starting from 30.0 g (115.68 mmol) of propan-2-yl 2-butylindolizine-7-carboxylate and 30.8 g (115.68 mmol) of 4-iodobenzoyl chloride and 14.84 g (114.82 mmol) of DIEA, 42.81 g of propan-2-yl 2-butyl-3-[(4-iodophenyl)carbonyl]indolizine-7-carboxylate are obtained in the form of a yellow solid, after silica column chromatography, elution being carried out with a cyclohexane/EtOAc gradient of 0 to 20% with respect to EtOAc.

Yield=76%

### 21.2 3-[(4-{[(3R)-1-(tert-Butoxycarbonyl)piperidin-3-yl]oxy}phenyl)carbonyl]-2-butylindolizine-7-carboxylate

A mixture of 8.0 g (16.35 mmol) of propan-2-yl 2-butyl-3-[(4-iodophenyl)carbonyl]indolizine-7-carboxylate,
6.0 g (29.81 mmol) of tert-butyl (3R)-3-hydroxypiperidine-1-carboxylate, 8.0 g (24.55 mmol) of Cs₂CO₃, 0.5 g (2.77 mmol) of 1,10-phenanthroline and 0.25 g (1.31 mmol) of CuI in 20 ml of anh. toluene is refluxed for 18 h under argon. The reaction mixture is then taken up with 200 ml of EtOAc, washed successively with 100 ml of water and 50 ml of brine, dried over Na₂SO₄, filtered, and then concentrated under reduced pressure. The residue obtained is chromatographed on a silica column, elution being carried out with a cyclohexane/EtOAc gradient of 0 to 40% with respect to EtOAc. After concentration under reduced pressure, 2.45 g of (3R)-1-(Cert-butoxycarbonyl)piperidin-3-yl 3-[(4-{[(3R)-1-(tert-butoxycarbonyl)piperidin-3-yl]oxy}phenyl)carbonyl]-2-butylindolizine-7-carboxylate are obtained in the form of a yellow powder.

Yield=21%.

### 21.3 3-[(4-{[(3R)-1-(tert-Butoxycarbonyl)piperidin-3-yl]oxy}phenyl)carbonyl]-2-butylindolizine-7-carboxylic acid

The process is carried out in the same way as in example 8.5. Thus, starting from 2.54 g (3.48 mmol) of (3R)-1-(tert-butoxycarbonyl)piperidin-3-yl 3-[(4-{[(3R)-1-(tert-butoxycarbonyl)piperidin-3-yl]oxy}phenyl)carbonyl]-2-butylindolizine-7-carboxylate, 0.65 g of 3-[(4-{[(3R)-1-(tert-butoxycarbonyl)piperidin-3-yl]oxy}phenyl)carbonyl]-2-butylindolizine-7-carboxylic acid is obtained in the form of a yellow foam.

Yield=36%.

### 21.4 tert-Butyl (3R)-3-[4-({2-butyl-7-[(2-methoxy-2-oxoethyl)(propan-2-yl)carbamoyl]indolizin-3-yl]carbonyl)phenoxy}piperidine-1-carboxylate

The process is carried out in the same way as in example 2.2. Thus, starting from 0.65 g (1.25 mmol) of 3-[(4-{[(3R)-1-(tert-butoxycarbonyl)piperidin-3-yl]oxy}phenyl)carbonyl]-2-butylindolizine-7-carboxylic acid, 0.31 g (1.87 mmol) of methyl N-propan-2-ylglycinate hydrochloride, 0.48 g (3.75 mmol) of DIEA and 0.60 g (1.87 mmol) of TBTU, 0.62 g of tert-butyl (3R)-3-[4-({2-butyl-7-[(2-methoxy-2-oxoethyl)(propan-2-yl)carbamoyl]indolizin-3-yl}carbonyl)phenoxy]-piperidine-1-carboxylate is obtained in the form of a yellow gum, after silica column chromatography, elution being carried out with a DCM/MeOH gradient of 0 to 10% with respect to MeOH.

Yield=78%.

### 21.5 Methyl N-{[2-butyl-3-({4-[(3R)-piperidin-3-yloxy]phenyl}carbonyl)indolizin-7-yl]carbonyl}-N-propan-2-ylglycinate hydrochloride

The process is carried out in the same way as in example 8.7. Thus, starting from 0.61 g (0.97 mmol) of tert-butyl (3R)-3-[4-({2-butyl-7-[(2-methoxy-2-oxoethyl)(propan-2-yl)carbamoyl]indolizin-3-yl]carbonyl)phenoxy}piperidine-1-carboxylate, 0.55 g of methyl N-{[2-butyl-3-({4-[(3R)-piperidin-3-yloxy]phenyl}carbonyl)indolizin-7-yl]carbonyl}-N-propan-2-ylglycinate hydrochloride is obtained in the form of a yellow powder.

Yield=100%.

Mp (° C.): 141.5

[a]_{D}²⁰=−2.6 (c=0.205; MeOH)

LC/MS: M=C₃₁H₃₉N₃O₅=533; M+H=534; Tr=1.09 min (conditions B)

¹H NMR (ppm, d₆-DMSO, 400 MHz):

9.35-9.25 (bs, 1H); 9.15-8.85 (bs, 2H); 7.70-7.65 (bs, 3H); 7.20 (d, 2H); 6.90-6.80 (bs, 1H); 6.65 (s, 1H); 4.95-4.85 (bs, 1H); 4.15 (s, 2H); 4.15-4.00 (bs, 1H); 3.75-3.60 (bs, 3H); 3.40-3.15 (bs, 2H); 3.15-3.05 (bs, 2H); 2.40-2.25 (bs, 2H); 2.00-1.80 (bs, 3H); 1.80-1.65 (bs, 1H); 1.50-1.35 (bs, 2H), 1.25-0.95 (bs, 8H); 0.80 (t, 3H).

The table which follows illustrates the chemical structures and the physical properties of some examples of compounds according to the invention:

TABLE 1

| | | | LCUVMS | |
|---|---|---|---|---|
| | | | RETEN- | MELTING |
| | | | TION | POINT |
| | | | TIME, | VALUE |
| No. | STRUCTURE | COMPOUND NAME | METHOD | (° C.) |
| 2 | 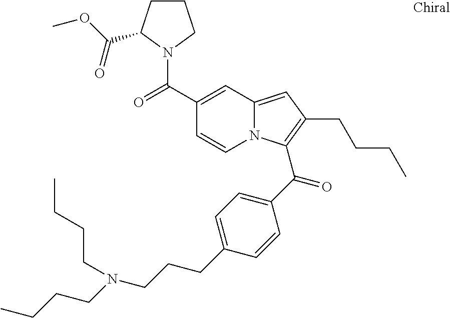 [SMILES: CCCCc1cc2cc(C(=O)N3CCC[C@H]3C(=O)OC)ccn2c1C(=O)c1ccc(CCCN(CCCC)CCCC)cc1 ; "Chiral"] | (S)-1-{2-Butyl-3-[4-(3- dibutylaminopropyl) benzoyl]indolizine-7- carbonyl}pyrrolidine-2- carboxylic acid methyl ester | 9 (A) | gum |
| 3 | 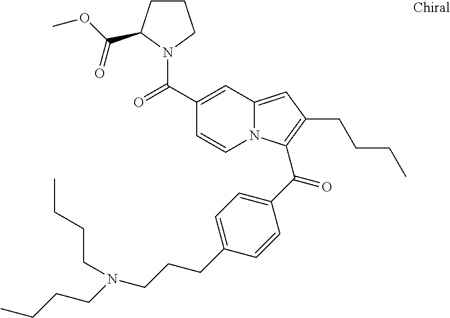 [SMILES: CCCCc1cc2cc(C(=O)N3CCC[C@@H]3C(=O)OC)ccn2c1C(=O)c1ccc(CCCN(CCCC)CCCC)cc1 ; "Chiral"] | (R)-1-{2-Butyl-3-[4-(3- dibutylaminopropyl) benzoyl]indolizine-7- carbonyl}pyrrolidine-2- carboxylic acid methyl ester | 9 (A) | gum |
| 4 | 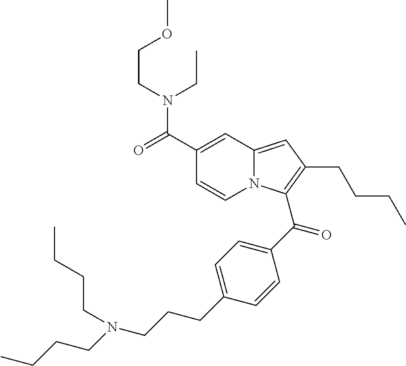 [SMILES: CCCCc1cc2cc(C(=O)N(CC)CCOC)ccn2c1C(=O)c1ccc(CCCN(CCCC)CCCC)cc1] | 2-Butyl-3-[4-(3- dibutylaminopropyl) benzoyl]indolizine-7- carboxylic acid ethyl (2-methoxyethyl)amide | 9.6 (A) | gum |
| 5 | 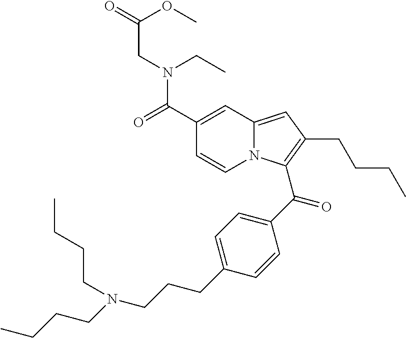 [SMILES: CCCCc1cc2cc(C(=O)N(CC)CC(=O)OC)ccn2c1C(=O)c1ccc(CCCN(CCCC)CCCC)cc1] | ({2-Butyl-3-[4-(3- dibutylaminopropyl)- benzoyl]indolizine-7- carbonyl}ethylamino)- acetic acid methyl ester | 9.3 (A) | gum |
| 6 | 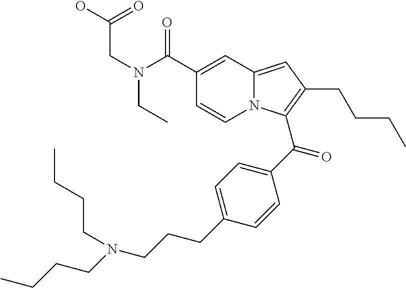 [SMILES: CCCCc1cc2cc(C(=O)N(CC)CC(=O)O)ccn2c1C(=O)c1ccc(CCCN(CCCC)CCCC)cc1] | ({2-Butyl-3-[4-(3- dibutylaminopropyl)- benzoyl]indolizine-7- carbonyl}ethylamino) acetic acid | 8.6 (A) | gum |
| 7 | 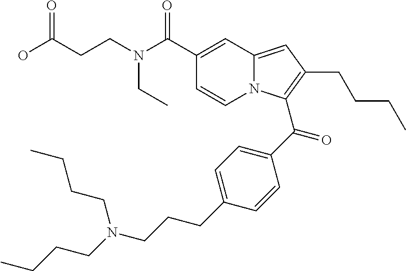 [SMILES: CCCCc1cc2cc(C(=O)N(CC)CCC(=O)O)ccn2c1C(=O)c1ccc(CCCN(CCCC)CCCC)cc1] | 3-({2-Butyl-3-[4-(3- dibutylaminopropyl)- benzoyl]indolizine-7- carbonyl}ethylamino)- propionic acid | 1.09 (B) | gum |
| 8 | 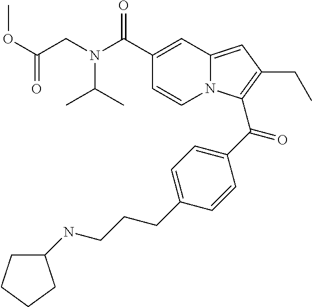 [SMILES: CCc1cc2cc(C(=O)N(CC(=O)OC)C(C)C)ccn2c1C(=O)c1ccc(CCCNC2CCCC2)cc1] | ({3-[4-(3- Cyclopentylaminopropyl) benzoyl]-2- ethylindolizine-7- carbonyl} isopropylamino)- acetic acid methyl ester | 1.09 (B) | 228 |
| 9 | 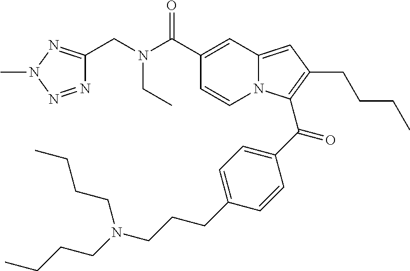 [SMILES: CCCCc1cc2cc(C(=O)N(CC)Cc3nnn(C)n3)ccn2c1C(=O)c1ccc(CCCN(CCCC)CCCC)cc1] | 2-Butyl-3-[4-(3- dibutylaminopropyl)- benzoyl]indolizine-7- carboxylic acid ethyl(2- methyl-2H-tetrazol-5- ylmethyl)amide | 1.16 (B) | gum |
| 10 | 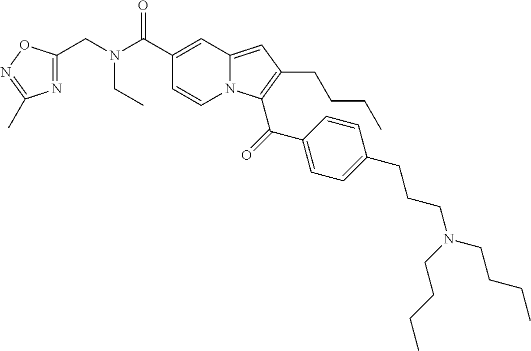 [SMILES: CCCCc1cc2cc(C(=O)N(CC)Cc3nc(C)no3)ccn2c1C(=O)c1ccc(CCCN(CCCC)CCCC)cc1] | 2-Butyl-3-[4-(3- dibutylaminopropyl)- benzoyl]indolizine-7- carboxylic acid ethyl(3- methyl[1,2,4]oxadiazol- 5-ylmethyl)amide | 1.18 (B) | gum |
| 11 | 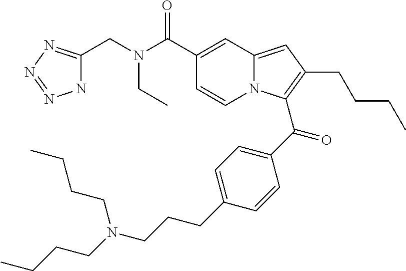 [SMILES: CCCCc1cc2cc(C(=O)N(CC)Cc3nnn[nH]3)ccn2c1C(=O)c1ccc(CCCN(CCCC)CCCC)cc1] | 2-Butyl-3-[4-(3- dibutylaminopropyl)- benzoyl]indolizine-7- carboxylic acid ethyl(1H-tetrazol-5- ylmethyl)amide | 3.82 (A) | gum |
| 12 | 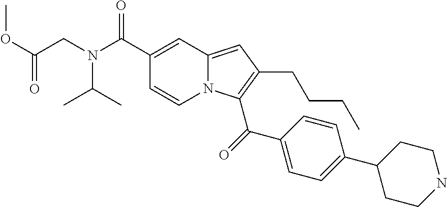 [SMILES: CCCCc1cc2cc(C(=O)N(CC(=O)OC)C(C)C)ccn2c1C(=O)c1ccc(C2CCNCC2)cc1] | {[2-Butyl-3-(4- piperidin-4-yl- benzoyl)indolizine- 7-carbonyl]- isopropylamino}acetic acid methyl ester | 0.99 (B) | 123 |
| 13 | 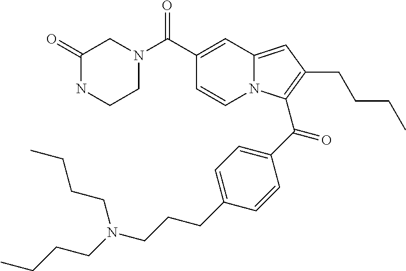 [SMILES: CCCCc1cc2cc(C(=O)N3CCNC(=O)C3)ccn2c1C(=O)c1ccc(CCCN(CCCC)CCCC)cc1] | 4-{2-Butyl-3-[4-(3- dibutylaminopropyl) benzoyl]indolizine-7- carbonyl}piperazin-2- one | 1.01 (B) | 162 |
| 14 | 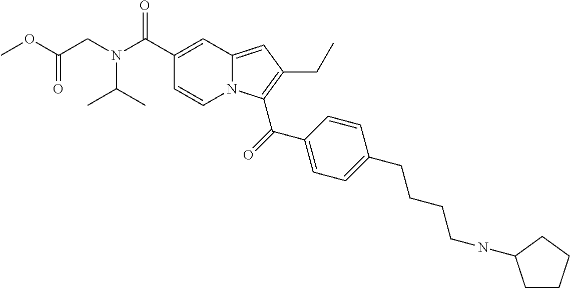 [SMILES: CCc1cc2cc(C(=O)N(CC(=O)OC)C(C)C)ccn2c1C(=O)c1ccc(CCCCNC2CCCC2)cc1] | ({3-[4-(4- Cyclopentylaminobutyl) benzoyl]-2- ethylindolizine-7- carbonyl} isopropylamino)- acetic acid methyl ester | 1.13 (B) | 154 |
| 15 | 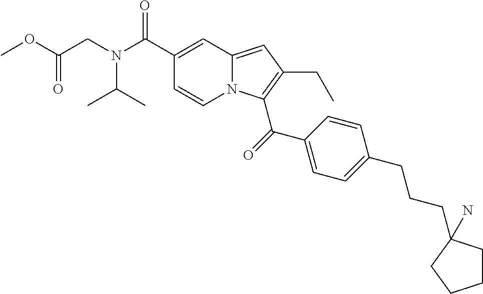 [SMILES: CCc1cc2cc(C(=O)N(CC(=O)OC)C(C)C)ccn2c1C(=O)c1ccc(CCCC2(N)CCCC2)cc1] | [(3-{4-[3-(1- Aminocyclopentyl) propyl]-benzoyl}-2- ethylindolizine-7- carbonyl)- isopropylamino]acetic acid methyl ester | 1.13 (B) | 176 |
| 16 | 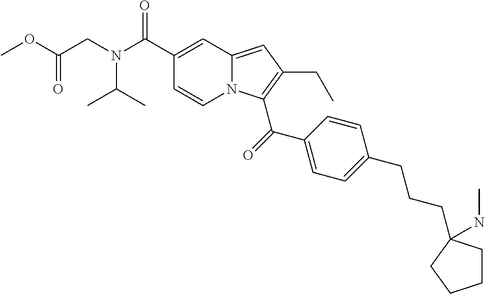 [SMILES: CCc1cc2cc(C(=O)N(CC(=O)OC)C(C)C)ccn2c1C(=O)c1ccc(CCCC2(NC)CCCC2)cc1] | [(2-Ethyl-3-{4-[3-(1- methylaminocyclopentyl)- propyl]benzoyl} indolizine-7-carbonyl)- isopropylamino]acetic acid methyl ester | 1.13 (B) | 149.5 |
| 17 | 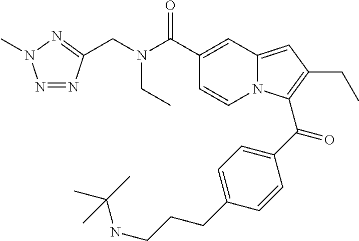 [SMILES: CCc1cc2cc(C(=O)N(CC)Cc3nnn(C)n3)ccn2c1C(=O)c1ccc(CCCNC(C)(C)C)cc1] | 3-[4-(3-tert- Butylaminopropyl) benzoyl]-2- ethylindolizine-7- carboxylic acid ethyl(2- methyl-2H-tetrazol- 5-ylmethyl)amide | 0.99 (B) | 172 |
| 18 | 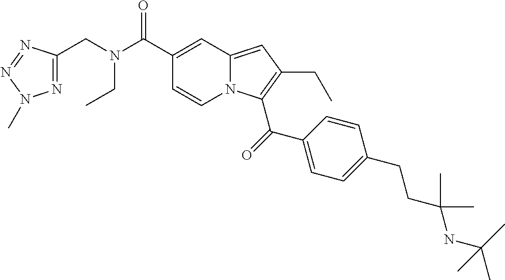 [SMILES: CCc1cc2cc(C(=O)N(CC)Cc3nnn(C)n3)ccn2c1C(=O)c1ccc(CCC(C)(C)NC(C)(C)C)cc1] | 3-[4-(3-tert-Butylamino- 3-methylbutyl)benzoyl]- 2-ethylindolizine-7- carboxylic acid ethyl(2- methyl-2H-tetrazol-5- ylmethyl)amide | 1.05 (B) | 122 |
| 19 | 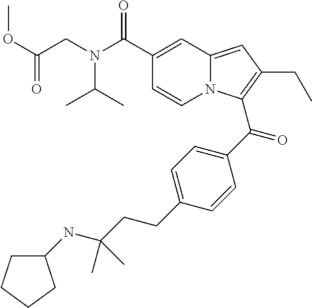 [SMILES: CCc1cc2cc(C(=O)N(CC(=O)OC)C(C)C)ccn2c1C(=O)c1ccc(CCC(C)(C)NC2CCCC2)cc1] | ({3-[4-(3- Cyclopentylamino-3- methylbutyl)benzoyl]- 2-ethylindolizine-7- carbonyl} isopropylamino) acetic acid methyl ester | 1.18 (B) | 245 |
| 20 | 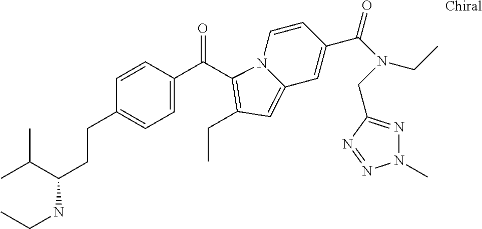 | 2-Ethyl-3-[4-((S)- 3-ethylamino-4- methylpentyl)benzoyl] indolizine-7-carboxylic acid ethyl(2-methyl- 2H-tetrazol-5- ylmethyl)amide | 1.05 (B) | 100 |
| 21 | 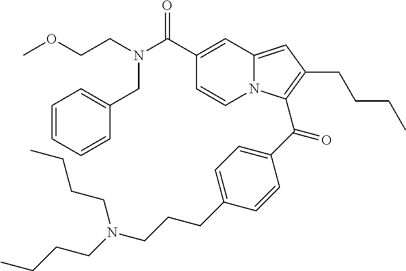 [SMILES: CCCCc1cc2cc(C(=O)N(CCOC)Cc3ccccc3)ccn2c1C(=O)c1ccc(CCCN(CCCC)CCCC)cc1] | 2-Butyl-3-[4-(3- dibutylaminopropyl) benzoyl]- indolizine-7-carboxylic acid benzyl(2- methoxyethyl)amide | 1.33 (B) | gum |
| 22 | 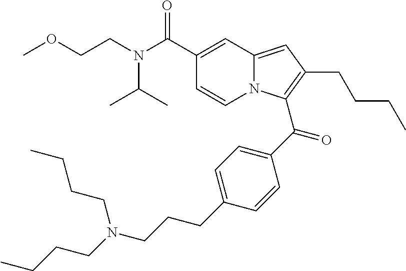 [SMILES: CCCCc1cc2cc(C(=O)N(CCOC)C(C)C)ccn2c1C(=O)c1ccc(CCCN(CCCC)CCCC)cc1] | 2-Butyl-3-[4-(3- dibutylaminopropyl) benzoyl]- indolizine-7-carboxylic acid isopropyl(2- methoxyethyl)amide | 1.26 (B) | gum |
| 23 | 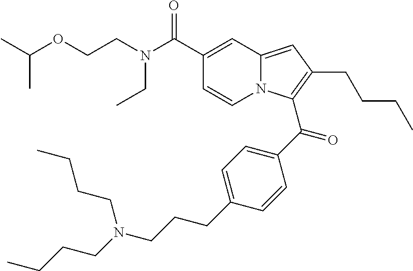 [SMILES: CCCCc1cc2cc(C(=O)N(CC)CCOC(C)C)ccn2c1C(=O)c1ccc(CCCN(CCCC)CCCC)cc1] | 2-Butyl-3-[4-(3- dibutylaminopropyl) benzoyl]- indolizine-7-carboxylic acid ethyl(2- isopropoxyethyl)amide | 1.29 (B) | gum |
| 24 | 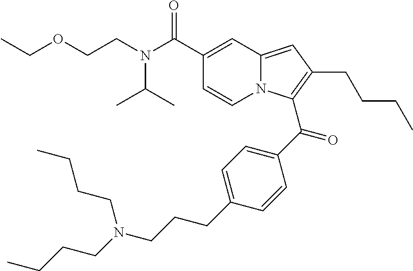 [SMILES: CCCCc1cc2cc(C(=O)N(CCOCC)C(C)C)ccn2c1C(=O)c1ccc(CCCN(CCCC)CCCC)cc1] | 2-Butyl-3-[4-(3- dibutylaminopropyl) benzoyl]- indolizine-7-carboxylic acid (2-ethoxyethyl)- isopropylamide | 1.32 (B) | gum |
| 25 | 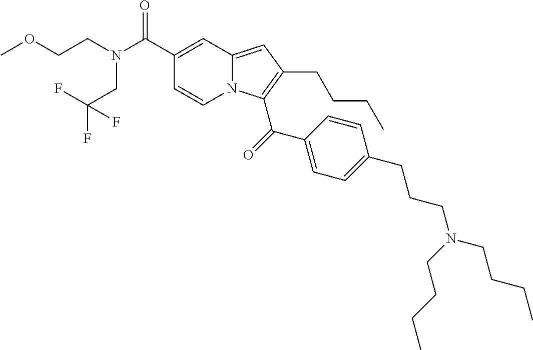 [SMILES: CCCCc1cc2cc(C(=O)N(CCOC)CC(F)(F)F)ccn2c1C(=O)c1ccc(CCCN(CCCC)CCCC)cc1] | 2-Butyl-3-[4-(3- dibutylaminopropyl) benzoyl]- indolizine-7-carboxylic acid (2- methoxyethyl)-(2,2,2- trifluoroethyl)amide | 5.67 (A) | gum |
| 26 | 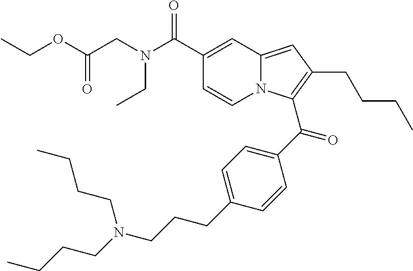 [SMILES: CCCCc1cc2cc(C(=O)N(CC)CC(=O)OCC)ccn2c1C(=O)c1ccc(CCCN(CCCC)CCCC)cc1] | ({2-Butyl-3-[4-(3- dibutylaminopropyl)- benzoyl]indolizine-7- carbonyl}ethylamino)- acetic acid ethyl ester | 1.22 (B) | gum |
| 27 | 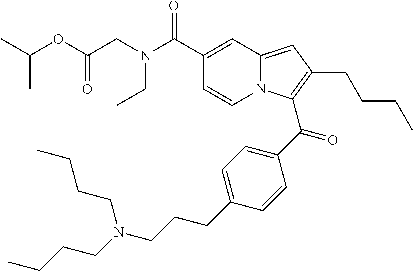 [SMILES: CCCCc1cc2cc(C(=O)N(CC)CC(=O)OC(C)C)ccn2c1C(=O)c1ccc(CCCN(CCCC)CCCC)cc1] | ({2-Butyl-3-[4-(3- dibutylaminopropyl)- benzoyl]indolizine-7- carbonyl}ethylamino)- acetic acid isopropyl ester | 1.27 (B) | gum |
| 28 | 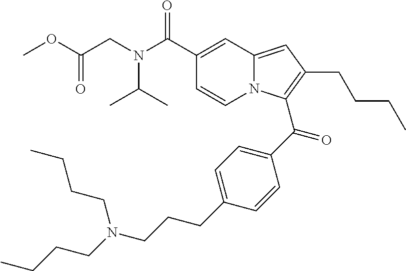 [SMILES: CCCCc1cc2cc(C(=O)N(CC(=O)OC)C(C)C)ccn2c1C(=O)c1ccc(CCCN(CCCC)CCCC)cc1] | ({2-Butyl-3-[4-(3- dibutylaminopropyl)- benzoyl]indolizine-7- carbonyl}- isopropylamino)acetic acid methyl ester | 1.22 (B) | gum |
| 29 | 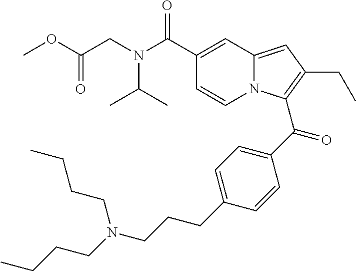 [SMILES: CCCCN(CCCC)CCCc1ccc(C(=O)c2c(CC)cc3cc(C(=O)N(CC(=O)OC)C(C)C)ccn23)cc1] | ({3-[4-(3- Dibutylaminopropyl) benzoyl]-2- ethylindolizine-7- carbonyl} isopropylamino)- acetic acid methyl ester | 1.13 (B) | 90 |
| 30 | 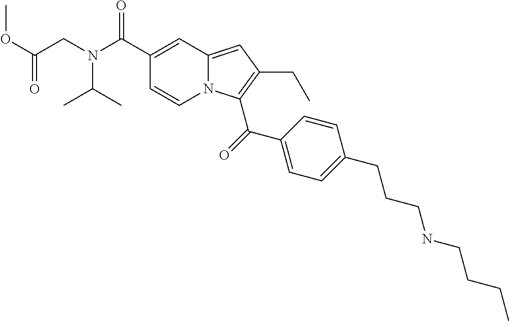 [SMILES: CCCCNCCCc1ccc(C(=O)c2c(CC)cc3cc(C(=O)N(CC(=O)OC)C(C)C)ccn23)cc1] | ({3-[4-(3- Butylaminopropyl) benzoyl]-2- ethylindolizine-7- carbonyl} isopropylamino)- acetic acid methyl ester | 1.01 (B) | 89 |
| 31 | 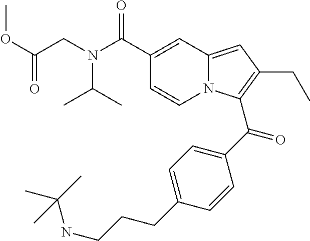 [SMILES: CCc1cc2cc(C(=O)N(CC(=O)OC)C(C)C)ccn2c1C(=O)c1ccc(CCCNC(C)(C)C)cc1] | ({3-[4-(3-tert- Butylaminopropyl) benzoyl]- 2-ethylindolizine-7- carbonyl} isopropylamino)- acetic acid methyl ester | 1.06 (B) | 204 |
| 32 | 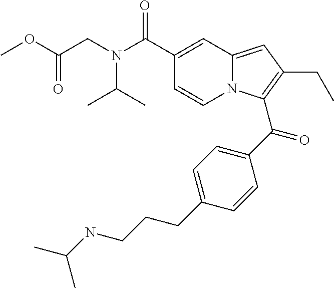 [SMILES: CCc1cc2cc(C(=O)N(CC(=O)OC)C(C)C)ccn2c1C(=O)c1ccc(CCCNC(C)C)cc1] | ({2-Ethyl-3-[4-(3- isopropylaminopropyl)- benzoyl]indolizine-7- carbonyl}isopropyl- amino)acetic acid methyl ester | 1.04 (B) | 290.4 |
| 33 | 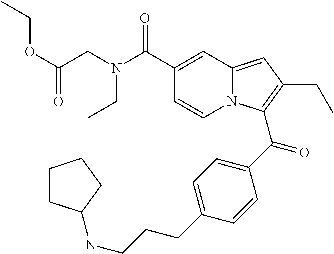 [SMILES: CCOC(=O)CN(CC)C(=O)c1ccn2c(C(=O)c3ccc(CCCNC4CCCC4)cc3)c(CC)cc2c1] | ({3-[4-(3- Cyclopentylaminopropyl) benzoyl]-2- ethylindolizine-7- carbonyl}ethylamino) acetic acid ethyl ester | 1.1 (B) | 153 |
| 34 | 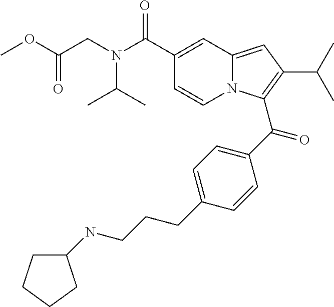 [SMILES: COC(=O)CN(C(=O)c1ccn2c(C(=O)c3ccc(CCCNC4CCCC4)cc3)c(C(C)C)cc2c1)C(C)C] | ({3-[4-(3- Cyclopentylaminopropyl) benzoyl]-2- isopropylindolizine-7- carbonyl}isopropyl- amino)acetic acid methyl ester | 1.12 (B) | 247 |
| 35 | 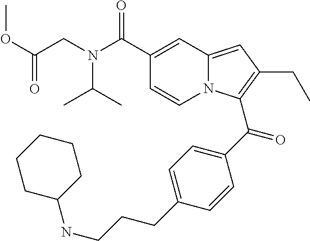 [SMILES: CCc1cc2cc(C(=O)N(CC(=O)OC)C(C)C)ccn2c1C(=O)c1ccc(CCCNC2CCCCC2)cc1] | ({3-[4-(3- Cyclohexylaminopropyl) benzoyl]-2- ethylindolizine-7- carbonyl}isopropyl- amino)acetic acid methyl ester | 1.17 (B) | 225 |
| 36 | 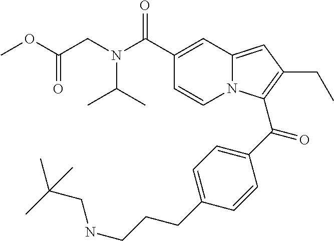 [SMILES: CCc1cc2cc(C(=O)N(CC(=O)OC)C(C)C)ccn2c1C(=O)c1ccc(CCCNCC(C)(C)C)cc1] | [(3-{4-[3-(2,2- Dimethylpropylamino) propyl]- benzoyl}-2- ethylindolizine-7- carbonyl) isopropylamino] acetic acid methyl ester | 1.12 (B) | 115 |
| 37 | 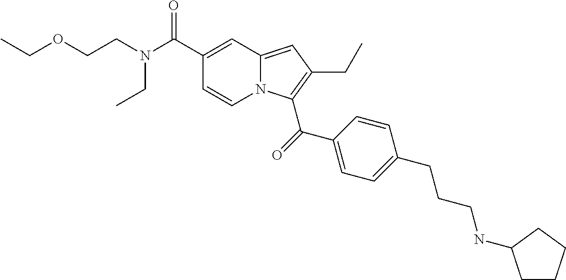 [SMILES: CCOCCN(CC)C(=O)c1ccn2c(C(=O)c3ccc(CCCNC4CCCC4)cc3)c(CC)cc2c1] | 3-[4-(3- Cyclopentylaminopropyl) benzoyl]-2- ethylindolizine-7- carboxylic acid (2- ethoxyethyl)ethyl amide | 1.12 (B) | 146.4 |
| 38 | 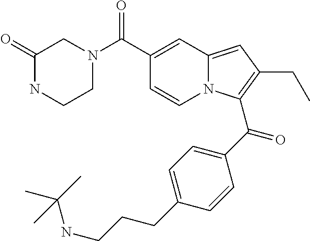 [SMILES: CCc1cc2cc(C(=O)N3CCNC(=O)C3)ccn2c1C(=O)c1ccc(CCCNC(C)(C)C)cc1] | 4-{3-[4-(3-tert- Butylaminopropyl) benzoyl]-2- ethylindolizine-7- carbonyl}piperazin- 2-one | 0.83 (B) | 122 |
| 39 | 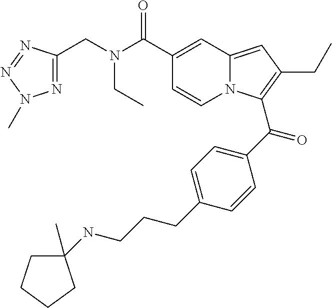 [SMILES: CCc1cc2cc(C(=O)N(CC)Cc3nnn(C)n3)ccn2c1C(=O)c1ccc(CCCNC2(C)CCCC2)cc1] | 2-Ethyl-3-{4-[3-(1- methylcyclopentylamino)- propyl]benzoyl} indolizine- 7-carboxylic acid ethyl(2-methyl-2H- tetrazol- 5-ylmethyl)amide | 1.06 (B) | 191.3 |
| 40 | 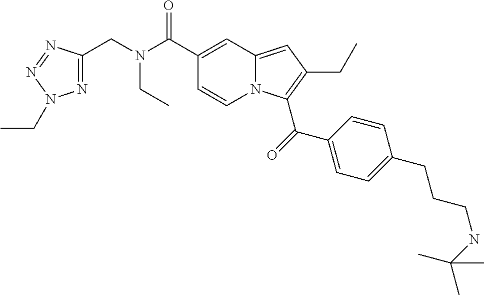 [SMILES: CCc1cc2cc(C(=O)N(CC)Cc3nnn(CC)n3)ccn2c1C(=O)c1ccc(CCCNC(C)(C)C)cc1] | 3-[4-(3-tert- Butylaminopropyl) benzoyl]-2- ethylindolizine-7- carboxylic acid ethyl(2- ethyl-2H-tetrazol-5- ylmethyl)amide | 1.01 (B) | 117 |
| 41 | 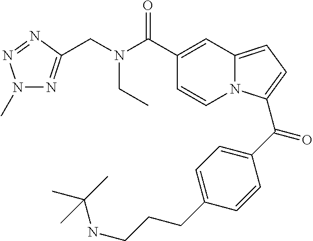 [SMILES: CCN(Cc1nnn(C)n1)C(=O)c1ccn2c(C(=O)c3ccc(CCCNC(C)(C)C)cc3)ccc2c1] | 3-[4-(3-tert- Butylaminopropyl) benzoyl]- indolizine-7-carboxylic acid ethyl(2-methyl- 2H-tetrazol-5-ylmethyl) amide | 0.81 (B) | 141.5 |
| 42 | 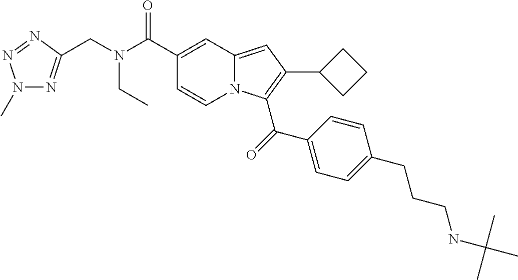 [SMILES: CCN(Cc1nnn(C)n1)C(=O)c1ccn2c(C(=O)c3ccc(CCCNC(C)(C)C)cc3)c(C3CCC3)cc2c1] | 3-[4-(3-tert- Butylaminopropyl) benzoyl]-2- cyclobutylindolizine-7- carboxylic acid ethyl(2-methyl-2H- tetrazol-5-ylmethyl) amide | 1.01 (B) | 195° C. |
| 43 | 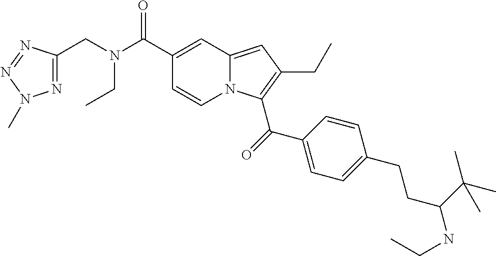 [SMILES: CCNC(CCc1ccc(C(=O)c2c(CC)cc3cc(C(=O)N(CC)Cc4nnn(C)n4)ccn23)cc1)C(C)(C)C] | 2-Ethyl-3-[4-(3- ethylamino-4,4- dimethylpentyl) benzoyl]indolizine-7- carboxylic acid ethyl (2-methyl-2H-tetrazol-5- ylmethyl)amide | | gum |
| 44 | 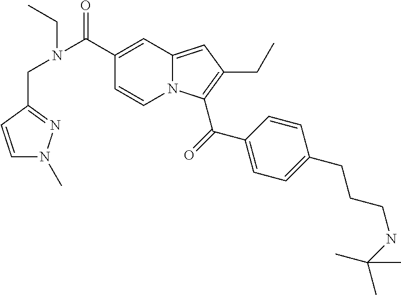 [SMILES: CCc1cc2cc(C(=O)N(CC)Cc3ccn(C)n3)ccn2c1C(=O)c1ccc(CCCNC(C)(C)C)cc1] | 3-[4-(3-tert- Butylaminopropyl) benzoyl]-2- ethylindolizine-7- carboxylic acid ethyl(1- methyl-1H-pyrazol- 3-ylmethyl)amide | 3.6 (B, 10 min) | 149 |
| 45 | 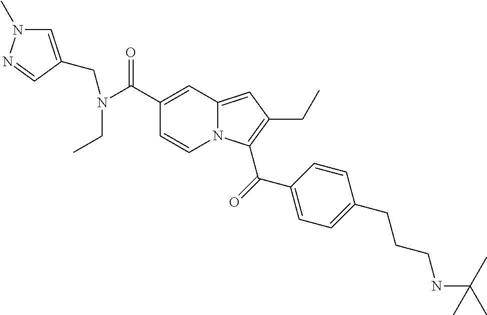 [SMILES: CCc1cc2cc(C(=O)N(CC)Cc3cnn(C)c3)ccn2c1C(=O)c1ccc(CCCNC(C)(C)C)cc1] | 3-[4-(3-tert- Butylaminopropyl) benzoyl]-2- ethylindolizine-7- carboxylic acid ethyl(1- methyl-1H-pyrazol- 4-ylmethyl)amide | 0.93 (B) | 173 |
| 46 | 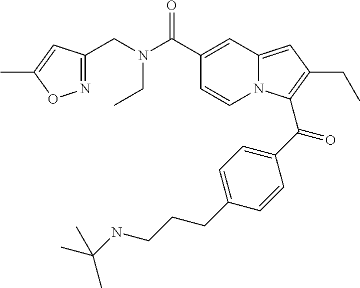 [SMILES: CCc1cc2cc(C(=O)N(CC)Cc3cc(C)on3)ccn2c1C(=O)c1ccc(CCCNC(C)(C)C)cc1] | 3-[4-(3-tert- Butylaminopropyl) benzoyl]-2- ethylindolizine-7- carboxylic acid ethyl(5- methylisoxazol-3- ylmethyl)amide | 1.01 (B) | 175 |
| 47 | 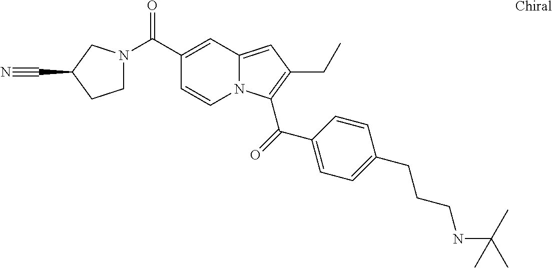 [SMILES: CCc1cc2cc(C(=O)N3CC[C@@H](C#N)C3)ccn2c1C(=O)c1ccc(CCCNC(C)(C)C)cc1 ; "Chiral"] | (R)-1-{3-[4-(3-tert- Butylaminopropyl)- benzoyl]-2- ethylindolizine-7- carbonyl}pyrrolidine- 3-carbonitrile | 0.96 (B) | 272 |
| 48 | 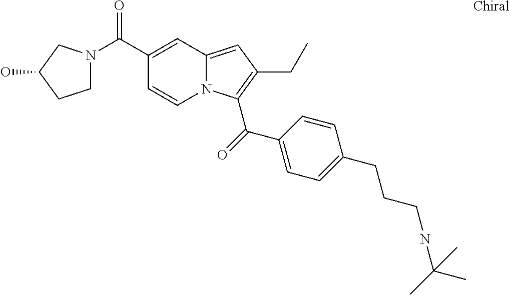 [SMILES: CCc1cc2cc(C(=O)N3CC[C@H](O)C3)ccn2c1C(=O)c1ccc(CCCNC(C)(C)C)cc1 ; "Chiral"] | {3-[4-(3-tert- Butylaminopropyl) benzoyl]-2- ethylindolizin-7-yl}- ((S)-3- hydroxypyrrolidin- 1-yl)methanone | 0.84 (B) | 124 |
| 49 | 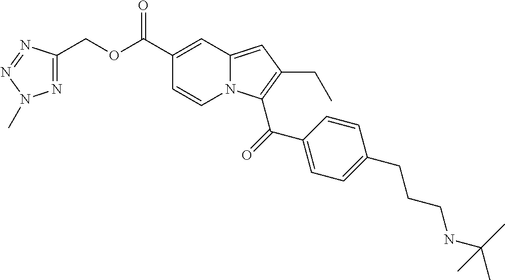 [SMILES: CCc1cc2cc(C(=O)OCc3nnn(C)n3)ccn2c1C(=O)c1ccc(CCCNC(C)(C)C)cc1] | 3-[4-(3-tert- Butylaminopropyl) benzoyl]-2- ethylindolizine-7- carboxylic acid 2- methyl- 2H-tetrazol-5- ylmethyl ester | 1.19 (B) | 203 |
| 50 | 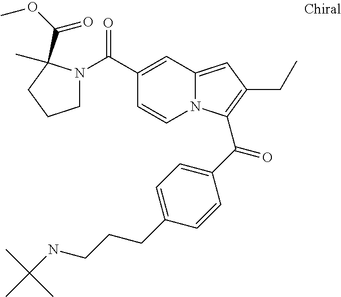 [SMILES: CCc1cc2cc(C(=O)N3CCC[C@@]3(C)C(=O)OC)ccn2c1C(=O)c1ccc(CCCNC(C)(C)C)cc1] | (S)-1-{3-[4-(3-tert- Butylaminopropyl)- benzoyl]-2- ethylindolizine-7- carbonyl}-2- methylpyrrolidine-2- carboxylic acid methyl ester | 1.13 (B) | 241 |
| 51 | 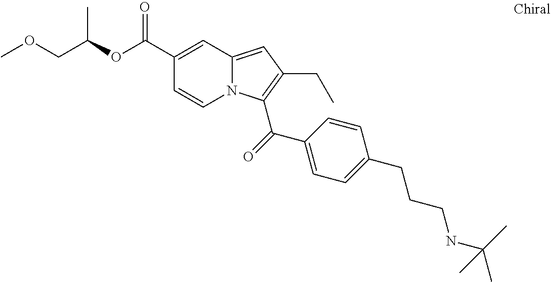 [SMILES: CCc1cc2cc(C(=O)O[C@H](C)COC)ccn2c1C(=O)c1ccc(CCCNC(C)(C)C)cc1] | 3-[4-(3-tert- Butylaminopropyl) benzoyl]-2- ethylindolizine-7- carboxylic acid (R)-2- methoxy-1-methylethyl ester | 1.37 (B) | 189 |
| 52 | 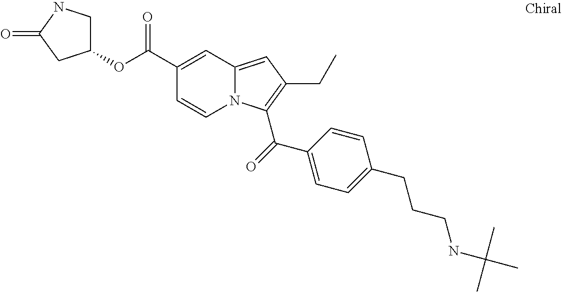 [SMILES: CCc1cc2cc(C(=O)O[C@H]3CNC(=O)C3)ccn2c1C(=O)c1ccc(CCCNC(C)(C)C)cc1] | 3-[4-(3-tert- Butylaminopropyl) benzoyl]-2- ethylindolizine-7- carboxylic acid (R)-5- oxopyrrolidin-3-yl ester | 0.82 (B) | 162 |
| 53 | 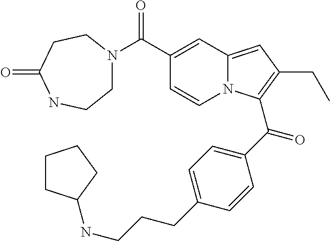 [SMILES: CCc1cc2cc(C(=O)N3CCNC(=O)CC3)ccn2c1C(=O)c1ccc(CCCNC2CCCC2)cc1] | 1-{3-[4-(3- Cyclopentylaminopropyl) benzoyl]-2- ethylindolizine-7- carbonyl}[1,4] diazepam-5-one | 0.87 (B) | 176 |
| 54 | 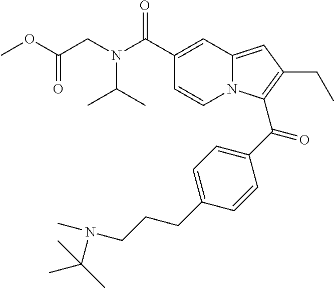 [SMILES: CCc1cc2cc(C(=O)N(CC(=O)OC)C(C)C)ccn2c1C(=O)c1ccc(CCCN(C)C(C)(C)C)cc1] | [(3-{4-[3-(tert- Butylmethylamino) propyl]- benzoyl}-2- ethylindolizine-7- carbonyl)isopropylamino] acetic acid methyl ester | 1.06 (B) | 252.1 |
| 55 | 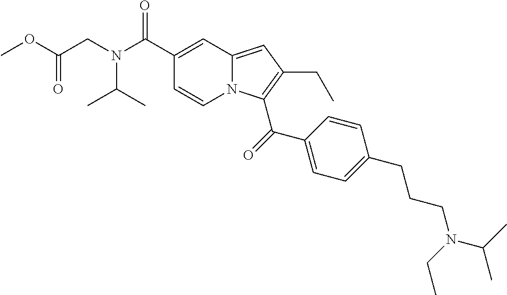 [SMILES: CCc1cc2cc(C(=O)N(CC(=O)OC)C(C)C)ccn2c1C(=O)c1ccc(CCCN(CC)C(C)C)cc1] | [(2-Ethyl-3-{4-[3- (ethylisopropylamino)- propyl]benzoyl} indolizine-7-carbonyl)- isopropylamino]acetic acid methyl ester | 1.08 (B) | 195.1 |
| 56 | 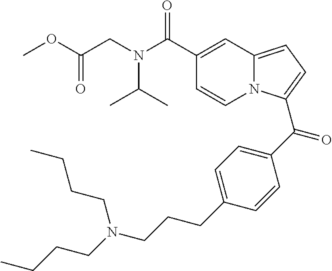 [SMILES: CCCCN(CCCC)CCCc1ccc(C(=O)c2ccc3cc(C(=O)N(CC(=O)OC)C(C)C)ccn23)cc1] | ({3-[4-(3- Dibutylaminopropyl) benzoyl]- indolizine-7-carbonyl} isopropylamino)acetic acid methyl ester | 1.08 (B) | 92 |
| 57 | 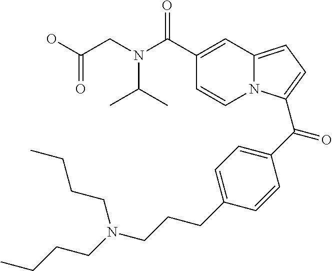 [SMILES: CCCCN(CCCC)CCCc1ccc(C(=O)c2ccc3cc(C(=O)N(CC(=O)O)C(C)C)ccn23)cc1] | ({3-[4-(3- Dibutylaminopropyl) benzoyl]- indolizine-7-carbonyl] isopropylamino)acetic acid | 0.98 (B) | gum |
| 58 | 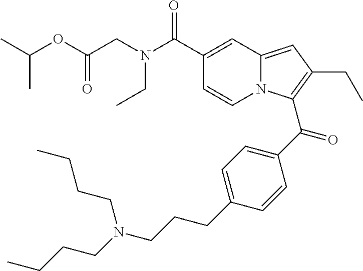 [SMILES: CCCCN(CCCC)CCCc1ccc(C(=O)c2c(CC)cc3cc(C(=O)N(CC)CC(=O)OC(C)C)ccn23)cc1] | ({3-[4-(3- Dibutylaminopropyl) benzoyl]-2- ethylindolizine-7- carbonyl}ethylamino) acetic acid isopropyl ester | 1.18 (B) | gum |
| 59 | 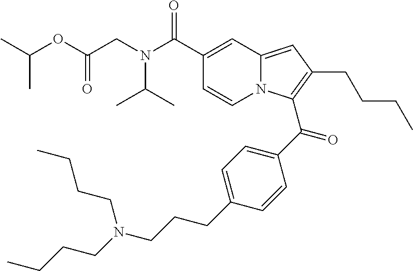 [SMILES: CCCCc1cc2cc(C(=O)N(CC(=O)OC(C)C)C(C)C)ccn2c1C(=O)c1ccc(CCCN(CCCC)CCCC)cc1] | ({2-Butyl-3-[4-(3- dibutylaminopropyl)- benzoyl]indolizine-7- carbonyl}- isopropylamino)acetic acid isopropyl ester | 1.3 (B) | gum |
| 60 | 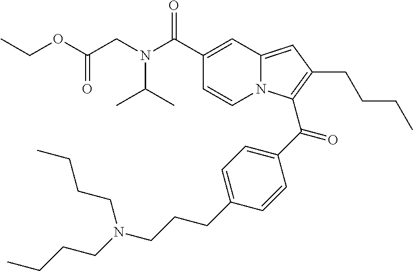 [SMILES: CCCCc1cc2cc(C(=O)N(CC(=O)OCC)C(C)C)ccn2c1C(=O)c1ccc(CCCN(CCCC)CCCC)cc1] | ({2-Butyl-3-[4-(3- dibutylaminopropyl)- benzoyl]indolizine-7- carbonyl}- isopropylamino)acetic acid ethyl ester | 1.27 (B) | gum |
| 61 | 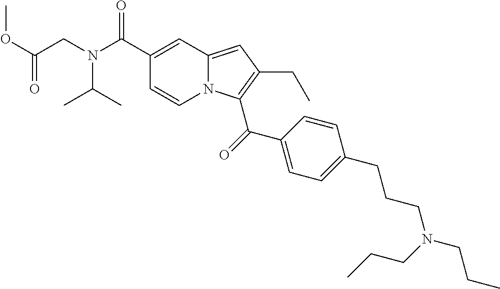 [SMILES: CCCN(CCC)CCCc1ccc(C(=O)c2c(CC)cc3cc(C(=O)N(CC(=O)OC)C(C)C)ccn23)cc1] | ({3-[4-(3- Dipropylaminopropyl) benzoyl]-2- ethylindolizine-7- carbonyl} isopropylamino)- acetic acid methyl ester | 1.05 (B) | gum |
| 62 | 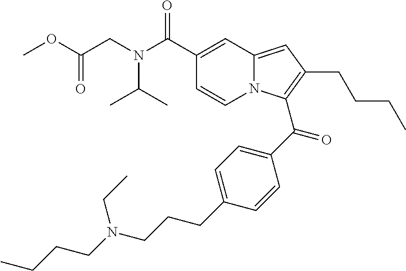 [SMILES: CCCCc1cc2cc(C(=O)N(CC(=O)OC)C(C)C)ccn2c1C(=O)c1ccc(CCCN(CC)CCCC)cc1] | [(2-Butyl-3-{4-[3- (butylethylamino)propyl]- benzoyl}indolizine-7- carbonyl)- isopropylamino]acetic acid methyl ester | 1.14 (B) | gum |
| 63 | 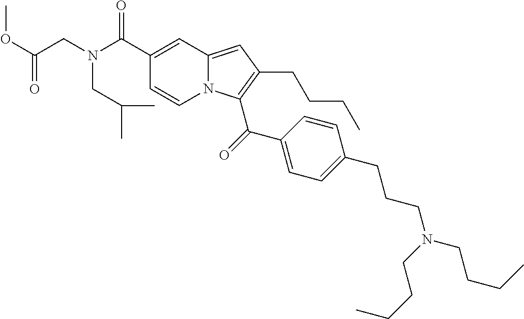 [SMILES: CCCCc1cc2cc(C(=O)N(CC(=O)OC)CC(C)C)ccn2c1C(=O)c1ccc(CCCN(CCCC)CCCC)cc1] | ({2-Butyl-3-[4-(3- dibutylaminopropyl)- benzoyl]indolizine-7- carbonyl}- isobutylamino)acetic acid methyl ester | 1.26 (B) | gum |
| 64 | 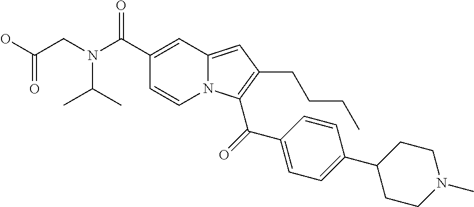 [SMILES: CCCCc1cc2cc(C(=O)N(CC(=O)O)C(C)C)ccn2c1C(=O)c1ccc(C2CCN(C)CC2)cc1] | ({2-Butyl-3-[4-(1- methylpiperidin-4- yl)benzoyl]indolizine- 7-carbonyl}- isopropylamino)acetic acid | 0.9 (B) | 193 |
| 65 | 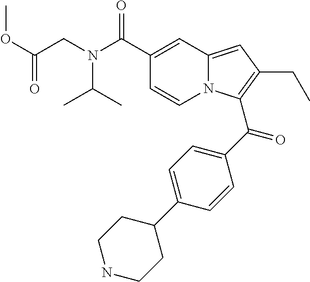 [SMILES: CCc1cc2cc(C(=O)N(CC(=O)OC)C(C)C)ccn2c1C(=O)c1ccc(C2CCNCC2)cc1] | {[2-Ethyl-3-(4- piperidin-4-yl- benzoyl)indolizine- 7-carbonyl]- isopropylamino}acetic acid methyl ester | 0.98 (B) | 234 |
| 66 | 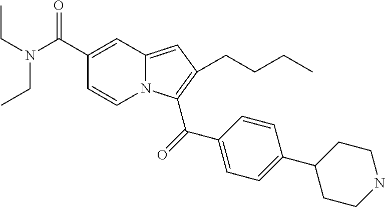 [SMILES: CCCCc1cc2cc(C(=O)N(CC)CC)ccn2c1C(=O)c1ccc(C2CCNCC2)cc1] | 2-Butyl-3-(4- piperidin-4-yl- benzoyl)indolizine-7- carboxylic acid diethylamide | 1.07 (B) | 149.6 |
| 67 | 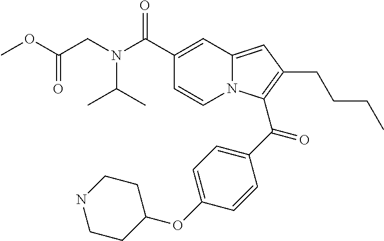 [SMILES: CCCCc1cc2cc(C(=O)N(CC(=O)OC)C(C)C)ccn2c1C(=O)c1ccc(OC2CCNCC2)cc1] | ({2-Butyl-3- [4-(piperidin-4- yloxy)benzoyl] indolizine-7-carbonyl}- isopropylamino)acetic acid methyl ester | 1.09 (B) | 147 |
| 68 | 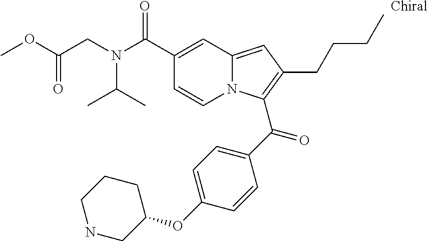 | ({2-Butyl-3-[4- ((S)-piperidin-3- yloxy)benzoyl] indolizine-7-carbonyl}- isopropylamino)acetic acid methyl ester | 1.09 (B) | 147.3 |
| 69 | 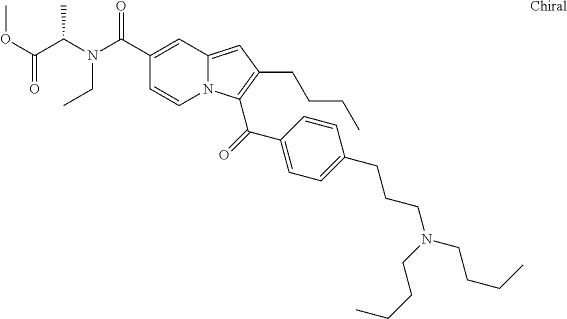 [SMILES: CCCCc1cc2cc(C(=O)N(CC)[C@@H](C)C(=O)OC)ccn2c1C(=O)c1ccc(CCCN(CCCC)CCCC)cc1 ; "Chiral"] | (S)-2-({2-Butyl-3-[4- (3-dibutylaminopropyl)- benzoyl]indolizine-7- carbonyl}ethylamino)- propionic acid methyl ester | 1.24 (B) | gum |
| 70 | 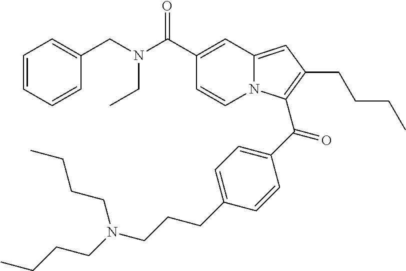 [SMILES: CCCCc1cc2cc(C(=O)N(CC)Cc3ccccc3)ccn2c1C(=O)c1ccc(CCCN(CCCC)CCCC)cc1] | 2-Butyl-3-[4-(3- dibutylaminopropyl) benzoyl]- indolizine-7-carboxylic acid benzylethyl amide | 1.34 (B) | gum |
| 71 | 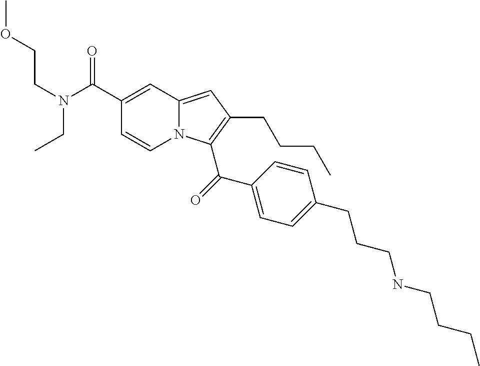 [SMILES: CCCCNCCCc1ccc(C(=O)c2c(CCCC)cc3cc(C(=O)N(CC)CCOC)ccn23)cc1] | 2-Butyl-3-[4-(3- butylaminopropyl) benzoyl]- indolizine-7-carboxylic acid ethyl(2- methoxyethyl)amide | 1.08 (B) | gum |
| 72 | 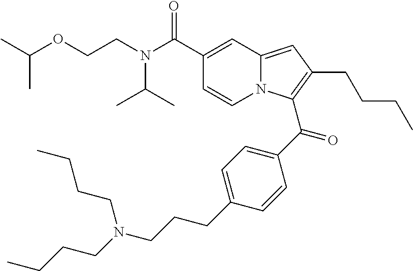 [SMILES: CCCCc1cc2cc(C(=O)N(CCOC(C)C)C(C)C)ccn2c1C(=O)c1ccc(CCCN(CCCC)CCCC)cc1] | 2-Butyl-3-[4-(3- dibutylaminopropyl) benzoyl]- indolizine-7- carboxylic acid (2- isopropoxyethyl) isopropylamide | 1.37 (B) | gum |
| 73 | 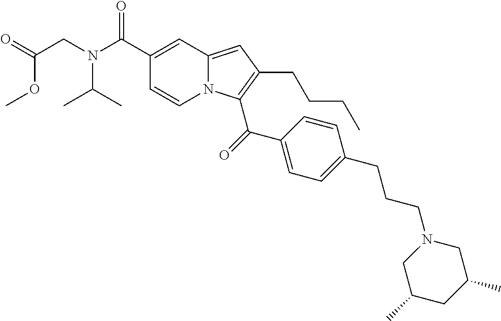 [SMILES: CCCCc1cc2cc(C(=O)N(CC(=O)OC)C(C)C)ccn2c1C(=O)c1ccc(CCCN2C[C@H](C)C[C@H](C)C2)cc1] | [(2-Butyl-3-{4-[3- ((3R,5S)-3,5- dimethylpiperidin- 1-yl)propyl]benzoyl}- indolizine-7-carbonyl) isopropylamino]acetic acid methyl ester | 1.14 (B) | 125 |
| 74 | 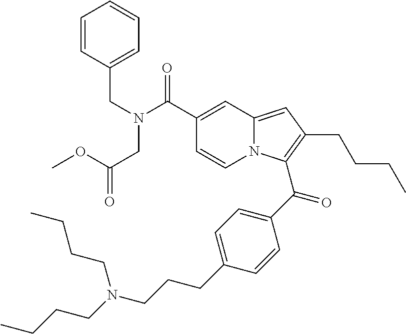 [SMILES: CCCCc1cc2cc(C(=O)N(CC(=O)OC)Cc3ccccc3)ccn2c1C(=O)c1ccc(CCCN(CCCC)CCCC)cc1] | (Benzyl-{2-butyl-3-[4- (3-dibutylaminopropyl)- benzoyl]indolizine-7- carbonyl}amino)acetic acid methyl ester | 1.3 (B) | gum |
| 75 | 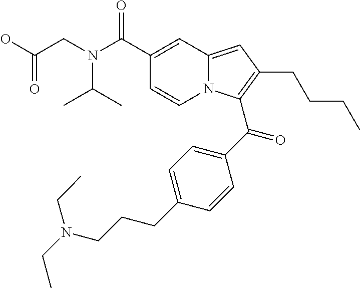 [SMILES: CCCCc1cc2cc(C(=O)N(CC(=O)O)C(C)C)ccn2c1C(=O)c1ccc(CCCN(CC)CC)cc1] | ({2-Butyl-3-[4-(3- diethylaminopropyl)- benzoyl]indolizine- 7-carbonyl}- isopropylamino)acetic acid | 0.97 (B) | gum |
| 76 | 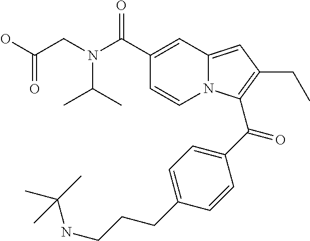 [SMILES: CCc1cc2cc(C(=O)N(CC(=O)O)C(C)C)ccn2c1C(=O)c1ccc(CCCNC(C)(C)C)cc1] | ({3-[4-(3-tert- Butylaminopropyl) benzoyl]-2- ethylindolizine-7- carbonyl} isopropylamino)- acetic acid | 0.97 (B) | 162 |
| 77 | 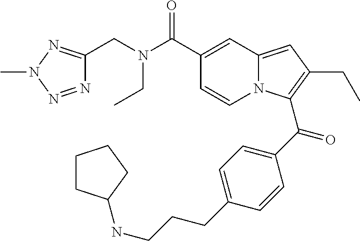 [SMILES: CCc1cc2cc(C(=O)N(CC)Cc3nnn(C)n3)ccn2c1C(=O)c1ccc(CCCNC2CCCC2)cc1] | 3-[4-(3- Cyclopentylaminopropyl) benzoyl]-2- ethylindolizine-7- carboxylic acid ethyl(2- methyl-2H-tetrazol- 5-ylmethyl)amide | 1.02 (B) | 104 |
| 78 | 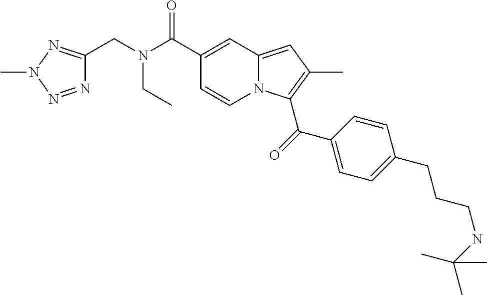 [SMILES: CCN(Cc1nnn(C)n1)C(=O)c1ccn2c(C(=O)c3ccc(CCCNC(C)(C)C)cc3)c(C)cc2c1] | 3-[4-(3-tert- Butylaminopropyl) benzoyl]-2- methylindolizine-7- carboxylic acid ethyl(2- methyl-2H-tetrazol- 5-ylmethyl)amide | 0.89 (B) | 153 |
| 79 | 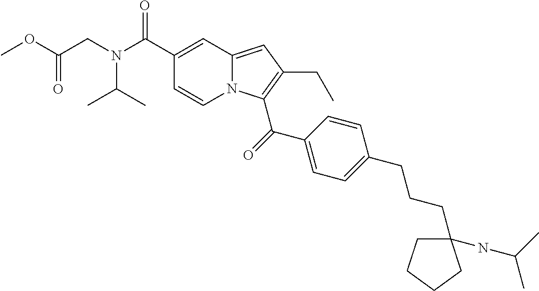 [SMILES: CCc1cc2cc(C(=O)N(CC(=O)OC)C(C)C)ccn2c1C(=O)c1ccc(CCCC2(NC(C)C)CCCC2)cc1] | [(2-Ethyl-3-{4-[3-(1- isopropylaminocyclo- pentyl)propyl]benzoyl} indolizine-7- carbonyl)isopropylamino] acetic acid methyl ester | 1.19 (B) | 190.7 |
| 80 | 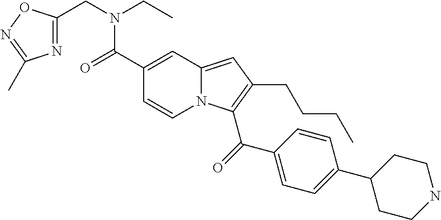 [SMILES: CCCCc1cc2cc(C(=O)N(CC)Cc3nc(C)no3)ccn2c1C(=O)c1ccc(C2CCNCC2)cc1] | 2-Butyl-3-(4-piperidin- 4-yl-benzoyl)- indolizine-7-carboxylic acid ethyl(3- methyl[1,2,4]oxadiazol- 5-ylmethyl)amide | 1.03 (B) | gum |
| 81 | 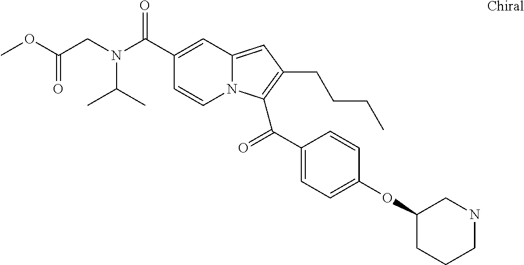 [SMILES: CCCCc1cc2cc(C(=O)N(CC(=O)OC)C(C)C)ccn2c1C(=O)c1ccc(O[C@@H]2CCCNC2)cc1 ; "Chiral"] | ({2-Butyl-3-[4- ((R)piperidin-3- yloxy)benzoyl] indolizine-7-carbonyl}- isopropylamino) acetic acid methyl ester | 1.09 (B) | 141.5 |

### Solubility

The evaluation of the solubility of the compounds of the invention is carried out at pH 4 (using a phosphate buffer, pH=6.01) by HPLC using an H₂O/CH₃CN/CH₃SO₃H gradient, relative to a reference sample (a dilute solution of the product to be evaluated which serves as an internal control). The solubility S results are expressed in mg/ml. Generally, the compounds of the present invention have a solubility S≧4 mg/ml at pH≧4. Among them, mention may be made of the solubilities of the following compounds in the table below:

TABLE 2

| | | | SOLU- | |
|---|---|---|---|---|
| | | | BILITY | pH |
| | | COMPOUND | RESULT | SOLU- |
| No. | STRUCTURE | NAME | (mg/ml) | BILITY |
| 2 | 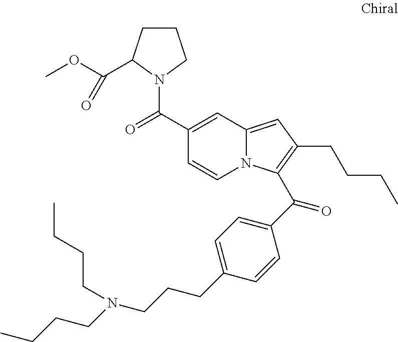 [SMILES: CCCCc1cc2cc(C(=O)N3CCCC3C(=O)OC)ccn2c1C(=O)c1ccc(CCCN(CCCC)CCCC)cc1] | (S)-1-{2- Butyl-3-[4-(3- dibutylamino- propyl)- benzoyl] indolizine-7- carbonyl} pyrrolidine-2- carboxylic acid methyl ester | 6.3 | 5.3 |
| 5 | 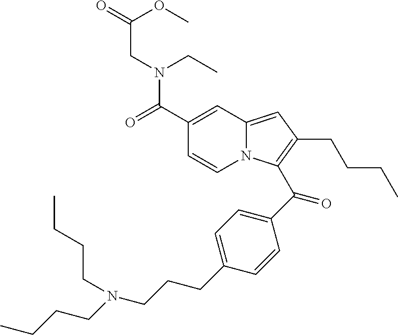 [SMILES: CCCCc1cc2cc(C(=O)N(CC)CC(=O)OC)ccn2c1C(=O)c1ccc(CCCN(CCCC)CCCC)cc1] | ({2-Butyl- 3-[4-(3- dibutylamino- propyl)- benzoyl] indolizine-7- carbonyl} ethylamino)- acetic acid methyl ester | 5.9 | 5.4 |
| 6 | 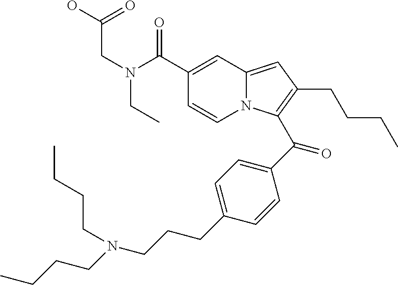 [SMILES: CCCCc1cc2cc(C(=O)N(CC)CC(=O)O)ccn2c1C(=O)c1ccc(CCCN(CCCC)CCCC)cc1] | ({2-Butyl- 3-[4-(3- dibutylamino- propyl)- benzoyl] indolizine-7- carbonyl} ethylamino)- acetic acid | 4.9 | 4.3 |
| 7 | 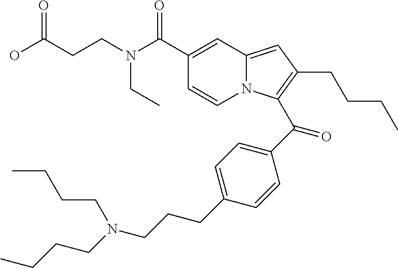 [SMILES: CCCCc1cc2cc(C(=O)N(CC)CCC(=O)O)ccn2c1C(=O)c1ccc(CCCN(CCCC)CCCC)cc1] | 3-({2-Butyl- 3-[4-(3- dibutylamino- propyl)- benzoyl] indolizine-7- carbonyl} ethylamino)- propionic acid | 7.2 | 6.2 |
| 8 | 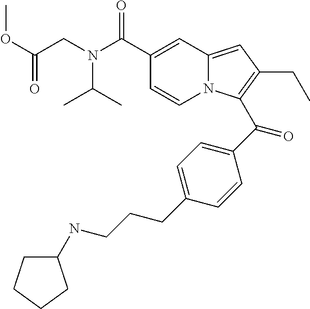 [SMILES: CCc1cc2cc(C(=O)N(CC(=O)OC)C(C)C)ccn2c1C(=O)c1ccc(CCCNC2CCCC2)cc1] | ({3-[4-(3- Cyclopentyl- amino- propyl) benzoyl]-2- ethylindolizine- 7-carbonyl} isopropylamino)- acetic acid methyl ester | 4.9 | 6.21 |
| 9 | 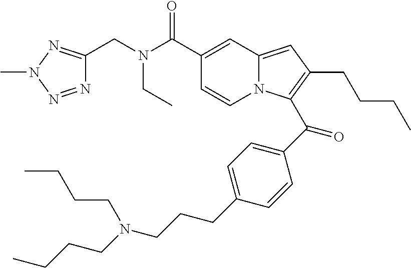 [SMILES: CCCCc1cc2cc(C(=O)N(CC)Cc3nnn(C)n3)ccn2c1C(=O)c1ccc(CCCN(CCCC)CCCC)cc1] | 2-Butyl- 3-[4-(3- dibutylamino- propyl)- benzoyl] indolizine-7- carboxylic acid ethyl(2- methyl-2H- tetrazol- 5-ylmethyl) amide | 7.22 | 5.82 |
| 10 | 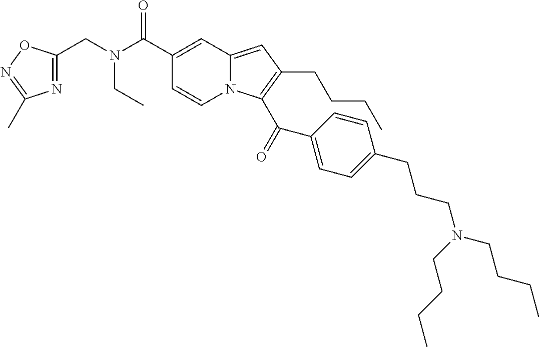 [SMILES: CCCCc1cc2cc(C(=O)N(CC)Cc3nc(C)no3)ccn2c1C(=O)c1ccc(CCCN(CCCC)CCCC)cc1] | 2-Butyl- 3-[4-(3- dibutylamino- propyl)- benzoyl] indolizine-7- carboxylic acid ethyl(3- methyl[1,2,4] oxadiazol- 5-ylmethyl) amide | 4.98 | 5.1 |
| 11 | 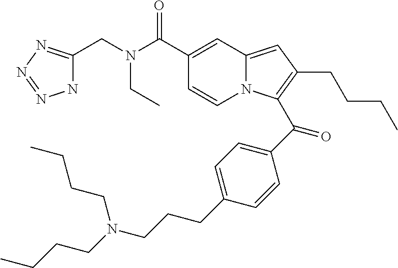 [SMILES: CCCCc1cc2cc(C(=O)N(CC)Cc3nnn[nH]3)ccn2c1C(=O)c1ccc(CCCN(CCCC)CCCC)cc1] | 2-Butyl- 3-[4-(3- dibutylamino- propyl)- benzoyl] indolizine-7- carboxylic acid ethyl(1H- tetrazol- 5-ylmethyl) amide | 0.24 | 5 |
| 16 | 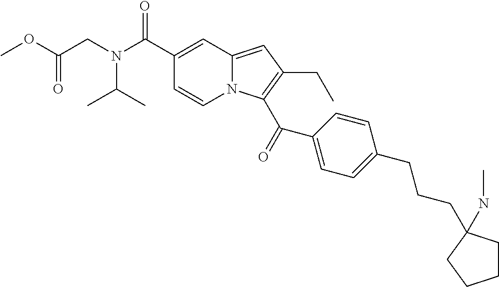 [SMILES: CCc1cc2cc(C(=O)N(CC(=O)OC)C(C)C)ccn2c1C(=O)c1ccc(CCCC2(NC)CCCC2)cc1] | [(2-Ethyl- 3-{4-[3-(1- methylamino- cyclopentyl)- propyl]benzoyl} indolizine-7- carbonyl)- isopropylamino] acetic acid methyl intenter | 0.63 | 5.38 |
| 17 | 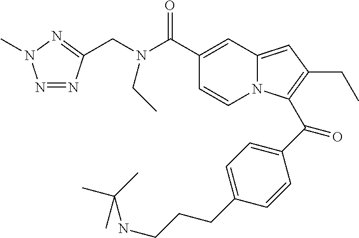 [SMILES: CCc1cc2cc(C(=O)N(CC)Cc3nnn(C)n3)ccn2c1C(=O)c1ccc(CCCNC(C)(C)C)cc1] | 3-[4-(3-tert- Butylamino- propyl) benzoyl]-2- ethylindolizine- 7- carboxylic acid ethyl(2- methyl- 2H-tetrazol- 5-ylmethyl) amide | 8.23 | 5.62 |
| 18 | 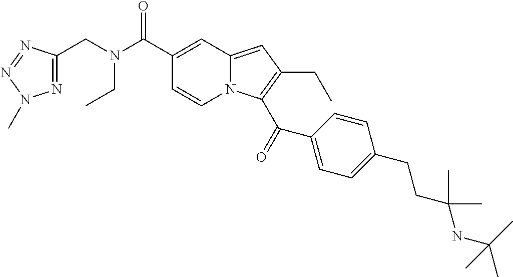 [SMILES: CCc1cc2cc(C(=O)N(CC)Cc3nnn(C)n3)ccn2c1C(=O)c1ccc(CCC(C)(C)NC(C)(C)C)cc1] | 3-[4-(3-tert- Butylamino- 3-methylbutyl)- benzoyl]-2- ethylindolizine- 7-carboxylic acid ethyl(2- methyl-2H- tetrazol-5- ylmethyl) amide | 7.88 | 6.1 |
| 19 | 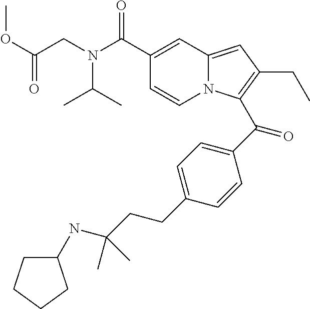 [SMILES: CCc1cc2cc(C(=O)N(CC(=O)OC)C(C)C)ccn2c1C(=O)c1ccc(CCC(C)(C)NC2CCCC2)cc1] | ({3-[4-(3- Cyclopentyl- amino- 3-methylbutyl) benzoyl]-2- ethylindolizine- 7-carbonyl}- isopropylamino) acetic acid methyl ester | 7.2 | 5.84 |
| 20 | 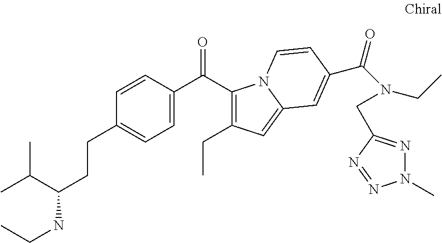 [SMILES: CCN[C@@H](CCc1ccc(C(=O)c2c(CC)cc3cc(C(=O)N(CC)Cc4nnn(C)n4)ccn23)cc1)C(C)C] | 2-Ethyl-3-[4- ((S)-3- ethylamino-4- methylpentyl) benzoyl] indolizine-7- carboxylic acid ethyl (2-methyl- 2H-tetrazol-5- ylmethyl) amide | 10.0 | 6.0 |
| 23 | 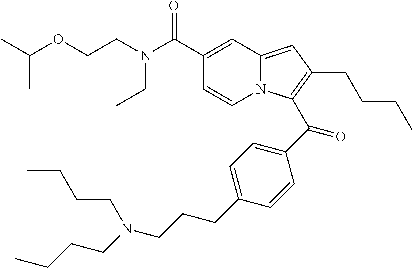 [SMILES: CCCCc1cc2cc(C(=O)N(CC)CCOC(C)C)ccn2c1C(=O)c1ccc(CCCN(CCCC)CCCC)cc1] | 2-Butyl- 3-[4-(3- dibutylamino- propyl) benzoyl]- indolizine-7- carboxylic acid ethyl(2- isopropoxyethyl) amide | 6.17 | 5.38 |
| 26 | 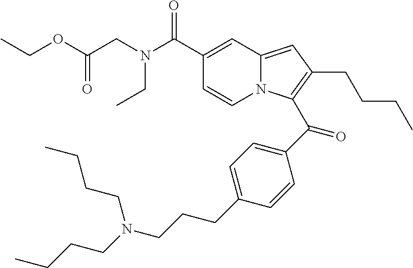 [SMILES: CCCCc1cc2cc(C(=O)N(CC)CC(=O)OCC)ccn2c1C(=O)c1ccc(CCCN(CCCC)CCCC)cc1] | ({2-Butyl- 3-[4-(3- dibutylamino- propyl)- benzoyl] indolizine-7- carbonyl} ethylamino)- acetic acid ethyl ester | 1.14 | 6.0 |
| 28 | 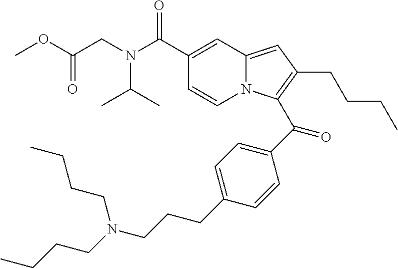 [SMILES: CCCCc1cc2cc(C(=O)N(CC(=O)OC)C(C)C)ccn2c1C(=O)c1ccc(CCCN(CCCC)CCCC)cc1] | ({2-Butyl- 3-[4-(3- dibutylamino- propyl)- benzoyl] indolizine- 7-carbonyl}- isopropylamino) acetic acid methyl ester | 2.42 | 5.9 |
| 30 | 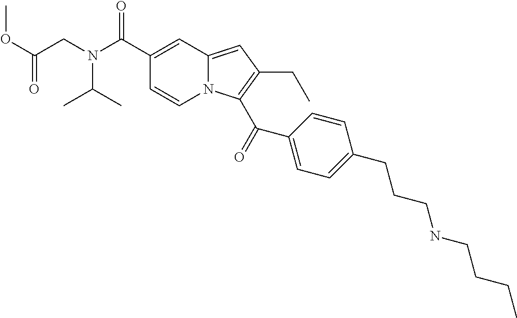 [SMILES: CCCCNCCCc1ccc(C(=O)c2c(CC)cc3cc(C(=O)N(CC(=O)OC)C(C)C)ccn23)cc1] | ({3-[4-(3- Butylamino- propyl) benzoyl]-2- ethylindolizine- 7- carbonyl} isopropylamino) acetic acid methyl ester | 10.0 | 6.05 |
| 31 | 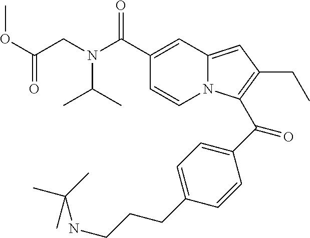 [SMILES: CCc1cc2cc(C(=O)N(CC(=O)OC)C(C)C)ccn2c1C(=O)c1ccc(CCCNC(C)(C)C)cc1] | ({3-[4-(3- Butylamino- propyl) benzoyl]-2- ethylindolizine- 7- carbonyl} isopropylamino)- acetic acid methyl ester | 2.8 | 6.08 |
| 32 | 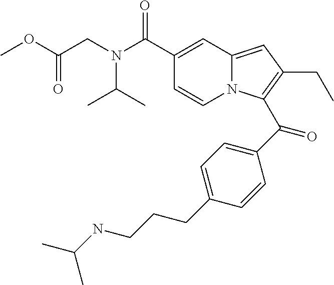 [SMILES: CCc1cc2cc(C(=O)N(CC(=O)OC)C(C)C)ccn2c1C(=O)c1ccc(CCCNC(C)C)cc1] | ({2-Ethyl- 3-[4-(3- isopropyl- aminopropyl)- benzoyl] indolizine- 7-carbonyl}- isopropylamino) acetic acid methyl ester | 10.0 | 6.3 |
| 35 | 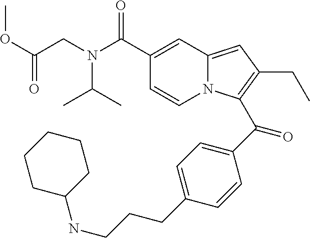 [SMILES: CCc1cc2cc(C(=O)N(CC(=O)OC)C(C)C)ccn2c1C(=O)c1ccc(CCCNC2CCCCC2)cc1] | ({3-[4-(3- Cyclohexyl- aminopropyl) benzoyl]-2- ethylindolizine- 7-carbonyl} isopropyl- amino)- acetic acid methyl ester | 1.57 | 6.3 |
| 37 | 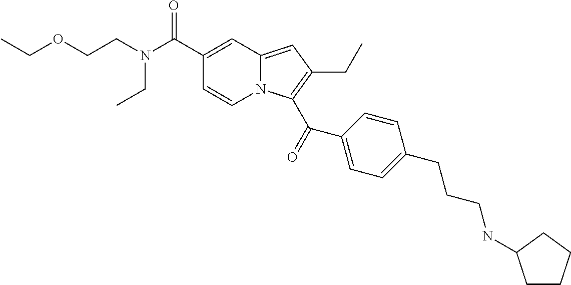 [SMILES: CCOCCN(CC)C(=O)c1ccn2c(C(=O)c3ccc(CCCNC4CCCC4)cc3)c(CC)cc2c1] | 3-[4-(3- Cyclopentyl- aminopropyl) benzoyl]-2- ethylindolizine- 7- carboxylic acid (2- ethoxyethyl) ethyl amide | 10.0 | 6.4 |
| 40 | 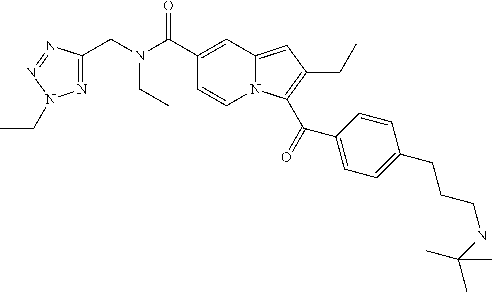 [SMILES: CCc1cc2cc(C(=O)N(CC)Cc3nnn(CC)n3)ccn2c1C(=O)c1ccc(CCCNC(C)(C)C)cc1] | 3-[4-(3-tert- Butylamino- propyl) benzoyl]-2- ethylindolizine- 7- carboxylic acid ethyl(2- ethyl-2H- tetrazol- 5-ylmethyl) amide | 10.0 | 5.54 |
| 44 | 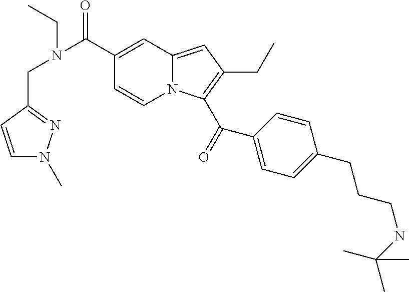 [SMILES: CCc1cc2cc(C(=O)N(CC)Cc3ccn(C)n3)ccn2c1C(=O)c1ccc(CCCNC(C)(C)C)cc1] | 3-[4-(3-tert- Butylamino- propyl) benzoyl]-2- ethylindolizine- 7- carboxylic acid ethyl(1- methyl-1H- pyrazol- 3-ylmethyl) amide | 10.0 | 6.0 |
| 45 | 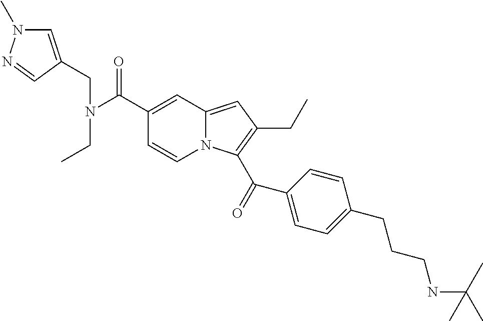 [SMILES: CCc1cc2cc(C(=O)N(CC)Cc3cnn(C)c3)ccn2c1C(=O)c1ccc(CCCNC(C)(C)C)cc1] | 3-[4-(3-tert- Butylamino- propyl) benzoyl]-2- ethylindolizine- 7- carboxylic acid ethyl(1- methyl-1H- pyrazol- 3-ylmethyl) amide | 10.0 | 6.0 |
| 46 | 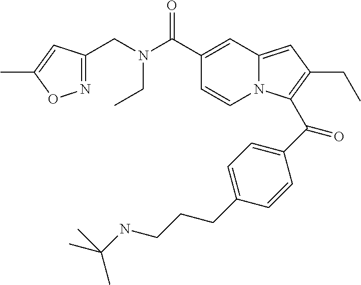 [SMILES: CCc1cc2cc(C(=O)N(CC)Cc3cc(C)on3)ccn2c1C(=O)c1ccc(CCCNC(C)(C)C)cc1] | 3-[4-(3-tert- Butylamino- propyl) benzoyl]-2- ethylindolizine- 7- carboxylic acid ethyl(5- methylisoxazol- 3-ylmethyl) amide | 1.73 | 6.0 |
| 50 | 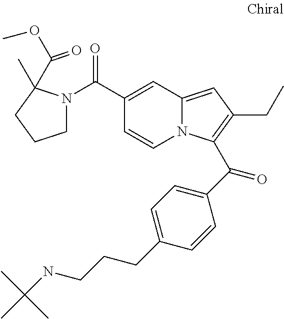 [SMILES: CCc1cc2cc(C(=O)N3CCCC3(C)C(=O)OC)ccn2c1C(=O)c1ccc(CCCNC(C)(C)C)cc1] | (S)-1-{3- [4-(3-tert- Butylamino- propyl) benzoyl]-2- ethylindolizine- 7-carbonyl}-2- methyl- pyrrolidine- 2-carboxylic acid methyl ester | 8.98 | 6.1 |
| 53 | 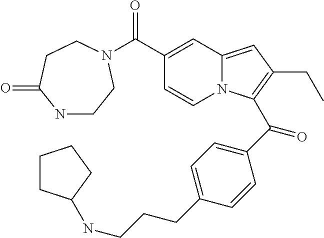 [SMILES: CCc1cc2cc(C(=O)N3CCNC(=O)CC3)ccn2c1C(=O)c1ccc(CCCNC2CCCC2)cc1] | 1-{3-[4-(3- Cyclopentyl- aminopropyl) benzoyl]-2- ethylindolizine- 7- carbonyl}[1,4] diazepam-5- one | 6.93 | 5.2 |
| 55 | 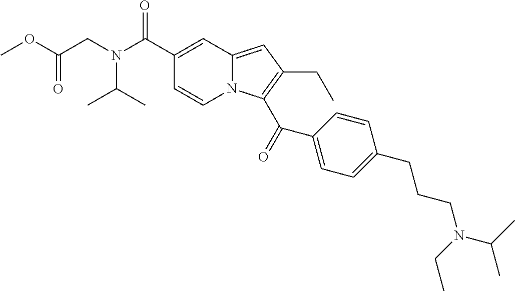 [SMILES: CCc1cc2cc(C(=O)N(CC(=O)OC)C(C)C)ccn2c1C(=O)c1ccc(CCCN(CC)C(C)C)cc1] | [(2-Ethyl- 3-{4-[3- (ethylisopropyl- amino)- propyl]benzoyl} indolizine-7- carbonyl)- isopropylamino] acetic acid methyl ester | 8.82 | 5.86 |
| 56 | 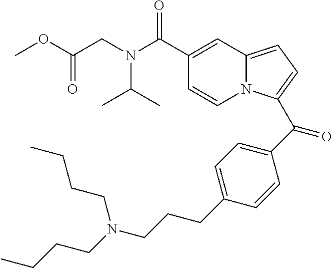 [SMILES: CCCCN(CCCC)CCCc1ccc(C(=O)c2ccc3cc(C(=O)N(CC(=O)OC)C(C)C)ccn23)cc1] | ({3-[4-(3- Dibutylamino- propyl) benzoyl]- indolizine-7- carbonyl} isopropylamino) acetic acid methyl ester | 8.3 | 6.0 |
| 58 | 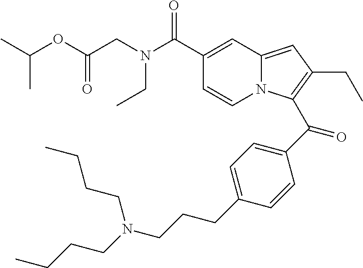 [SMILES: CCCCN(CCCC)CCCc1ccc(C(=O)c2c(CC)cc3cc(C(=O)N(CC)CC(=O)OC(C)C)ccn23)cc1] | ({3-[4-(3- Dibutylamino- propyl) benzoyl]-2- ethylindolizine-7- carbonyl} ethylamino) acetic acid isopropyl ester | 6.3 | 5.7 |
| 59 | 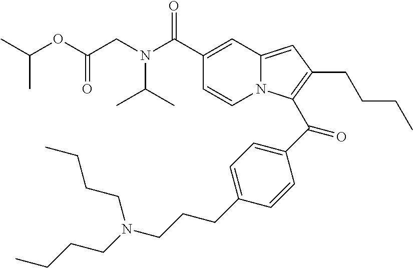 [SMILES: CCCCc1cc2cc(C(=O)N(CC(=O)OC(C)C)C(C)C)ccn2c1C(=O)c1ccc(CCCN(CCCC)CCCC)cc1] | ({2-Butyl- 3-[4-(3- dibutylamino- propyl)- benzoyl] indolizine- 7-carbonyl}- isopropylamino) acetic acid isopropyl ester | 5.7 | 5.4 |
| 61 | 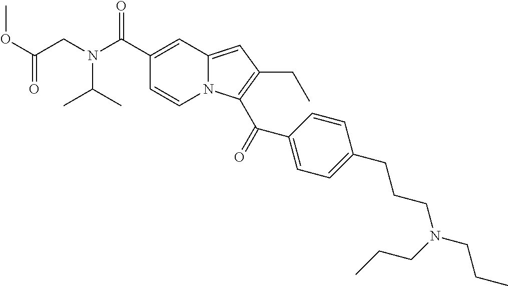 [SMILES: CCCN(CCC)CCCc1ccc(C(=O)c2c(CC)cc3cc(C(=O)N(CC(=O)OC)C(C)C)ccn23)cc1] | ({3-[4-(3- Dipropylamino- propyl) benzoyl]-2- ethylindolizine- 7- carbonyl} isopropylamino)- acetic acid methyl ester | 8.93 | 6.4 |
| 62 | 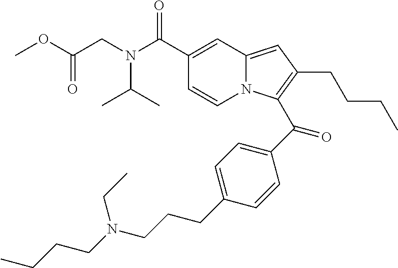 [SMILES: CCCCc1cc2cc(C(=O)N(CC(=O)OC)C(C)C)ccn2c1C(=O)c1ccc(CCCN(CC)CCCC)cc1] | [(2-Butyl- 3-{4-[3- butylethyl- amino) propyl]- benzoyl} indolizine- 7-carbonyl)- isopropylamino] acetic acid methyl ester | 5.3 | 6.18 |
| 69 | 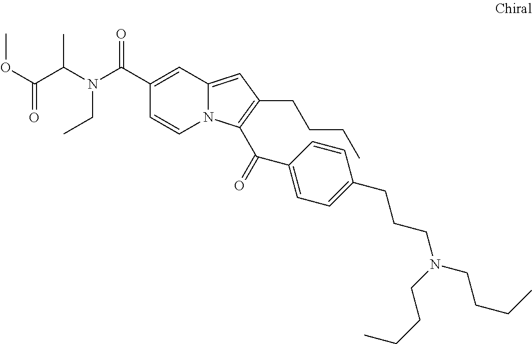 [SMILES: CCCCc1cc2cc(C(=O)N(CC)C(C)C(=O)OC)ccn2c1C(=O)c1ccc(CCCN(CCCC)CCCC)cc1] | (S)-2-({2- Butyl-3-[4- (3- dibutylamino- propyl)- benzoyl] indolizine-7- carbonyl} ethylamino)- propionic acid methyl ester | 5.72 | 5.6 |
| 75 | 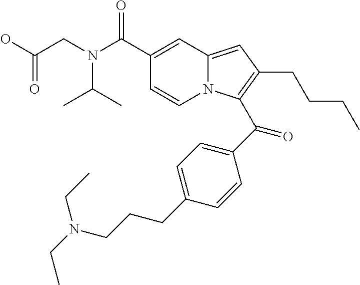 [SMILES: CCCCc1cc2cc(C(=O)N(CC(=O)O)C(C)C)ccn2c1C(=O)c1ccc(CCCN(CC)CC)cc1] | ({2-Butyl- 3-[4-(3- diethylamino- propyl)- benzoyl] indolizine-7- carbonyl}- isopropylamino) acetic acid | 8.4 | 10.14 |
| 76 | 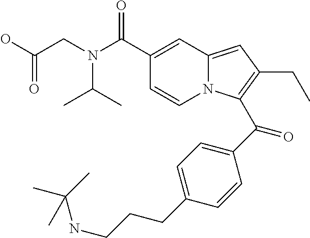 [SMILES: CCc1cc2cc(C(=O)N(CC(=O)O)C(C)C)ccn2c1C(=O)c1ccc(CCCNC(C)(C)C)cc1] | ({3-[4-(3-tert- Butylamino- propyl) benzoyl]-2- ethylindolizine- 7- carbonyl} isopropyl- amino)- acetic acid | 6.2 | 4.12 |
| 79 | 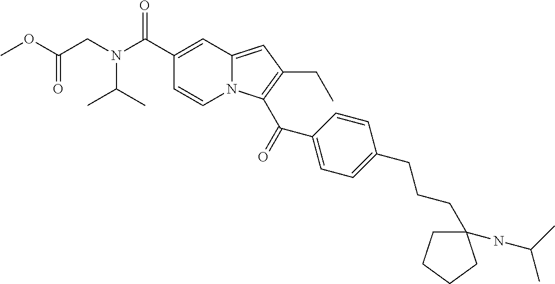 [SMILES: CCc1cc2cc(C(=O)N(CC(=O)OC)C(C)C)ccn2c1C(=O)c1ccc(CCCC2(NC(C)C)CCCC2)cc1] | [(2-Ethyl- 3-{4- [3-(1- isopropyl- amino- cyclopentyl) propyl] benzoyl} indolizine-7- carbonyl) isopropyl- amino] acetic acid methyl ester | 8.02 | 5.99 |
| 80 | 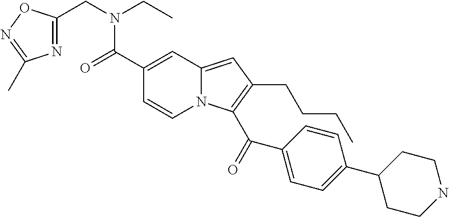 [SMILES: CCCCc1cc2cc(C(=O)N(CC)Cc3nc(C)no3)ccn2c1C(=O)c1ccc(C2CCNCC2)cc1] | 2-Butyl- 3-(4- piperidin-4- ylbenzoyl)- indolizine-7- carboxylic acid ethyl(3- methyl[1,2,4] oxadiazol- 5-ylmethyl) amide | 4.2 | 5.9 |
| 81 | 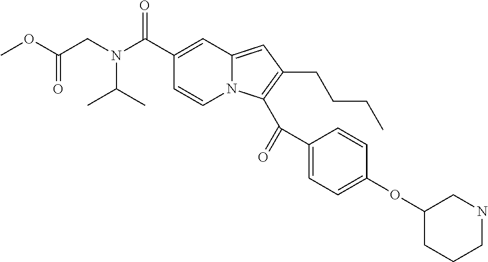 [SMILES: CCCCc1cc2cc(C(=O)N(CC(=O)OC)C(C)C)ccn2c1C(=O)c1ccc(OC2CCCNC2)cc1] | ({2-Butyl- 3-[4- ((R)piperidin- 3-yloxy)- benzoyl] indolizine- 7-carbonyl}- isopropyl- amino) acetic acid methyl ester | 10 | 6.19 |

### Effect of the Compounds on Left Atrial Fibrillation

The effect of the compounds of the invention on atrial refraction and on the induction of brief episodes of atrial fibrillation/flutter caused by premature atrial beats of the left atrium was studied in pigs of the German Landrace strain, anesthetized with pentobarbital and subjected to a thoractomy (Knobloch et al. Electrophysiological and antiarrhythmic effects of the novel IKur channel blockers, 59947 and 520951, on left vs. right pig atrium in vivo in comparison with the IKr blockers dofetilide, azimilide, d,I-sotalol and ibutilide”, Naunyn-Schmiedeberg's Arch Pharmacol 2002, 366: 482-487). Left atrial vulnerability in this pig model is also a valid parameter for testing the efficacy of atrial antiarrhythmic compounds and has demonstrated its predictiveness in humans (Knobloch et al.).

### Method

The animals were premedicated with 2 ml of intramuscular (i.m.) Rompun® 2% and 2 ml of Zoletil100°, and anesthetized with 5 ml of Narcoren© (pentobarbital, 160 mg/ml=25-30 mg/kg i.v.) injected as an intravenous (i.v.) bolus, followed by a continuous intravenous drip of pentobarbital at 12-17 mg/kg/h. The heart was exposed after left thoracotomy supported by a pericardial cradle. The animals are ventilated by respiratory assistance (air/oxygen). The analysis of blood gases (pO₂; pCO₂) was carried out at regular intervals in order to check the oxygen supply provided by the respirator and to maintain a pO₂>100 mmHg and a pCO₂<35 mmHg.

To record the hemodynamic parameters, electronic catheters of Millar PC 350 type are implanted in the left femoral artery (BPs/d: abbreviation for blood pressure systolic/diastolic), the pulmonary artery and in the left ventricular via the right carotid artery (LVP, LVEDP and HR: abbreviations for left ventricular pressure, left ventricular end-diastolic pressure and heart rate, respectively).

The bipolar surface ECGs (electrocardiograms) were recorded by means of lead II or III needle electrodes implanted subcutaneously.

A monophasic action potential electrode is placed in the right atrium via a venous approach, and another on the epicardium of the left atrium in order to measure the atrial refraction.

The electrophysiological data are continuously recorded and stored on the hard disk of a computer via an online acquisition and analysis system (Hem Notocord Evolution, Croissy-sur-Seine, France).

The left and right atrial refractions are measured according to the S1-S2 incrementation protocol with base cycle lengths of 240, 300 and 400 ms before and after administration of the vehicle or of the test compound at regular intervals (15, 30, 60, 90, 120 min).

The episodes of brief atrial fibrillations which frequently follow the premature beat S2 are noted and compared with the base recording (left atrial vulnerability: maximum 45 min before and after the injection of the test compound).

The evaluation of the QT interval was carried out during right atrial pacing, the rate of which was increased by 10 beats per minute compared with the sinus rate for the first 15 minutes after the administration so as to avoid having to correct the duration of the QT interval and the monophasic action potential (MAP) relative to the heart rate. For this purpose, a stimulating electrode is placed on the proximal part of the left atrium. This procedure makes it possible to distinguish the compounds which affect the ventricular repolarization (prolonged QT interval is an undesirable effect, since it promotes ventricular arrhythmias).

The electrophysiological recording (ECG and MAP) makes it possible to identify the standard side effects which are often related to the blockage of potassium, sodium and calcium channels in the heart (prolonged QT, atrioventicular block, delayed conduction). Hemodynamic monitoring makes it possible to distinguish the adverse effects relating to inappropriate blockage of potassium channels [increase in arterial pressure (AP) and in pulmonary pressure (PP)], and of sodium and calcium channels (negative inotropic effects, drop in arterial pressure).

### Results:

The compound of the invention is evaluated on 2 to 4 pigs at 3 mg/kg i.v., bolus or as a drip for 15 min, and with 3 pacing rates (150, 200 and 250 bpm). The results obtained are expressed as % increase in right and left atrial refractory periods (LAERP and RAERP, respectively), and as % decrease in left atrial vulnerability (episodes of atrial fibrillation induced by S2, LAV) relative to the base line, and the duration of action is expressed in hours (h). Compounds No. 2 to 81 of the invention, which prolong the LAERP by at least 20%, inhibit the LAV by at least 60%, prolong the QTc by a maximum of 5 ms and induce a negative inotropic effect of a maximum of 20% or an increase in AP or in PP of a maximum of 5 mmHg.

It therefore appears that the compounds according to the invention have an advantageous pharmacological activity, in particular antiarrhythmic properties.

The compounds according to the invention can therefore be used for preparing medicaments, in particular antiarrhythmic medicaments.

Thus, according to another of its aspects, a subject of the invention is medicaments which comprise a compound of formula (I), or an addition salt thereof with a pharmaceutically acceptable acid of the compound of formula (I).

These medicaments are of therapeutic use in particular in the treatment and prevention of atrial and ventricular arrhythmias: atrial tachyarrhythmia, atrial fibrillation, atrial flutter, atrial tachycardia, ventricular tachyarrhythmia, ventricular extrasystoles, ventricular tachycardia, ventricular flutter and fibrillation; of angina pectoris, of hypertension, of cerebral circulatory insufficiency, of heart failure, of myocardium infarction which may or may not be complicated by heart failure, or the prevention of post-infarction mortality, or of stroke.

Thus, according to another of its aspects, a subject of the invention is the use for a compound of formula (I) for preparing a medicament intended for the treatment of pathological syndromes of the cardiovascular system.

According to another of its aspects, the present invention relates to pharmaceutical compositions comprising, as active ingredient, a compound according to the invention. These pharmaceutical compositions contain an effective dose of at least one compound according to the invention, or a pharmaceutically acceptable salt of said compound, and also at least one pharmaceutically acceptable excipient.

Said excipients are chosen, according to the pharmaceutical form and the method of administration desired, from the usual excipients which are known to those skilled in the art.

In the pharmaceutical compositions of the present invention for oral, sublingual, subcutaneous, intramuscular, intravenous, topical, local, intratracheal, intranasal, transdermal or rectal administration, the active ingredient of formula (I) above, or salt thereof, can be administered in a unit administration form, as a mixture with conventional pharmaceutical excipients, to animals and to human beings for the treatment of the above disorders or diseases.

The suitable unit administration forms include oral administration forms, such as tablets, soft or hard capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms, and implants. For topical application, the compounds according to the invention can be used in creams, gels, ointments or lotions.

By way of example, a unit administration form of a compound according to the invention in tablet form can comprise the following constituents:

| | | |
|---|---|---|
| Compound according to the invention | 50.0 | mg |
| Mannitol | 223.75 | mg |
| Sodium croscaramellose | 6.0 | mg |
| Corn starch | 15.0 | mg |
| Hydroxypropylmethylcellulose | 2.25 | mg |
| Magnesium stearate | 3.0 | mg |

There may be specific cases where higher or lower dosages are appropriate; such dosages do not part from the context of the invention. According to the usual practice, the dosage appropriate for each patient is determined by the physician according to the method of administration and the weight and response of said patient.

According to another of its aspects, the present invention also relates to a method for treating the pathological conditions indicated above, which comprises the administration, to a patient, of an effective dose of a compound according to the invention, or a pharmaceutically acceptable salt thereof.

1. A compound corresponding to formula (I) 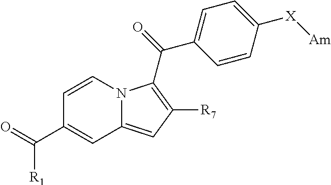
wherein
R1 represents:
either 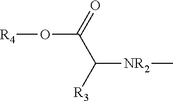
or 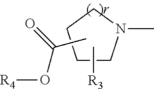
or 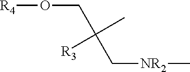
or 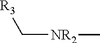
or 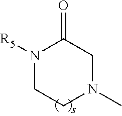
or 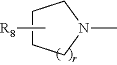
or 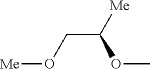
or 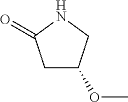
or 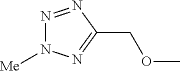
R2 represents a hydrogen atom, a (C₁-C₆) alkyl group, a benzyl group or a CH₂—CF₃ group;
R3 represents a hydrogen atom, a (C₁-C₆) alkyl group or a benzyl group;
R4 represents a hydrogen atom or a (C₁-C₄) alkyl group;
R5 represents a hydrogen atom or a (C₁-C₅) alkyl group;
R6 represents a nitrile group or a heteroaryl group comprising from 1 to 4 heteroatoms chosen from a nitrogen atom and an oxygen atom, this heteroaryl group being optionally substituted with a (C₁-C₆) alkyl group;
R7 represents a hydrogen atom or a linear, branched or cyclic (C₁-C₆) alkyl group;
R8 represents a hydroxyl group or a cyano group;
X represents a bond or an oxygen atom;
Am represents:
either 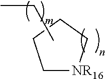
or
(CH₂)ₜ—CR₁₉R₂₀NR₁₇—R₁₈
R16 represents a hydrogen atom or a (C₁-C₆) alkyl group;
R17 represents a hydrogen atom or a (C₁-C₆) alkyl group;
R18 represents a branched or cyclic (C₁-C₆) alkyl group;
R19 and R20 represent a hydrogen atom or a (C₁-C₆) alkyl group, or form a (C₃-C₄) spiroalkyl group;
m represents an integer equal to 0 or 1;
n represents an integer equal to 1 or 2;
r represents an integer equal to 1 or 2;
s represents an integer equal to 1 or 2;
t represents an integer between 2 and 4;
in the form of a base or an addition salt with an acid.

2. The compound of formula (I) as claimed in claim 1, chosen from:
compound No. 2: (S)-1-[2-Butyl-3-[4-(3-dibutylamino-propyl)benzoyl]indolizine-7-carbonyl]-pyrrolidine-2-carboxylic acid methyl ester;
compound No. 3: (R)-1-{2-Butyl-3-[4-(3-dibutylamino-propyl)benzoyl]indolizine-7-carbonyl}pyrrolidine-2-carboxylic acid methyl ester;
compound No. 4: 2-Butyl-3-[4-(3-dibutylaminopropyl)benzoyl]indolizine-7-carboxylic acid ethyl (2-methoxyethyl)amide;
compound No. 5: ({2-Butyl-3-[4-(3-dibutyl-aminopropyl)benzoyl]indolizine-7-carbonyl}ethylamino)acetic acid methyl ester;
compound No. 6: ({2-Butyl-3-[4-(3-dibutyl-aminopropyl)benzoyl]indolizine-7-carbonyl}ethylamino)acetic acid;
compound No. 7: 3-({2-Butyl-3-[4-(3-dibutyl-aminopropyl)benzoyl]indolizine-7-carbonyl}ethylamino)propionic acid;
compound No. 8: ({3-[4-(3-Cyclopentylaminopropyl)benzoyl]-2-ethylindolizine-7-carbonyl}isopropylamino)acetic acid methyl ester;
compound No. 9: 2-Butyl-3-[4-(3-dibutylamino-propyl)benzoyl]indolizine-7-carboxylic acid ethyl(2-methyl-2H-tetrazol-5-ylmethyl)amide;
compound No. 10: 2-Butyl-3-[4-(3-dibutylamino-propyl)benzoyl]indolizine-7-carboxylic acid ethyl(3-methyl-[1,2,4]oxadiazol-5-ylmethyl)amide;
compound No. 11: 2-Butyl-3-[4-(3-dibutylamino-propyl)benzoyl]indolizine-7-carboxylic acid ethyl(1H-tetrazole-5-ylmethyl)amide;
compound No. 12: {[2-Butyl-3-(4-piperidin-4-ylbenzoyl)indolizine-7-carbonyl]isopropylamino}acetic acid methyl ester;
compound No. 13: 4-{2-Butyl-3-[4-(3-dibutylamino-propyl)benzoyl]indolizine-7-carbonyl}piperazine-2-one;
compound No. 14: ({3-[4-(4-Cyclopentylamino-butyl)benzoyl]-2-ethylindolizine-7-carbonyl}isopropylamino)acetic acid methyl ester;
compound No. 15: [(3-{4-[3-(1-Aminocyclopentyl)propyl]benzoyl}-2-ethylindolizine-7-carbonyl)isopropylamino]acetic acid methyl ester;
compound No. 16: [(2-Ethyl-3-{4-[3-(1-methyl-aminocyclopentyl)propyl]benzoyl}indolizine-7-carbonyl)isopropylamino]acetic acid methyl ester;
compound No. 17: 3-[4-(3-tert-Butylaminopropyl)benzoyl]-2-ethylindolizine-7-carboxylic acid ethyl(2-methyl-2H-tetrazol-5-ylmethyl)amide;
compound No. 18: 3-[4-(3-tert-Butylamino-3-methyl-butyl)benzoyl]-2-ethylindolizine-7-carboxylic acid ethyl(2-methyl-2H-tetrazol-5-ylmethyl)amide;
compound No. 19: ({3-[4-(3-Cyclopentylamino-3-methyl-butyl)benzoyl]-2-ethylindolizine-7-carbonyl}isopropylamino)acetic acid methyl ester;
compound No. 20: 2-Ethyl-3-[4-((S)-3-ethylamino-4-methylpentyl)benzoyl]indolizine-7-carboxylic acid ethyl(2-methyl-2H-tetrazol-5-ylmethyl)amide;
compound No. 21: 2-Butyl-3-[4-(3-dibutylamino-propyl)benzoyl]indolizine-7-carboxylic acid benzyl(2-methoxyethyl)amide;
compound No. 22: 2-Butyl-3-[4-(3-dibutylamino-propyl)benzoyl]indolizine-7-carboxylic acid isopropyl(2-methoxyethyl)amide;
compound No. 23: 2-Butyl-3-[4-(3-dibutylamino-propyl)benzoyl]indolizine-7-carboxylic acid ethyl(2-isopropoxyethyl)amide;
compound No. 24: 2-Butyl-3-[4-(3-dibutylamino-propyl)benzoyl]indolizine-7-carboxylic acid (2-ethoxyethyl)isopropylamide;
compound No. 25: 2-Butyl-3-[4-(3-dibutylaminopropyl)benzoyl]indolizine-7-carboxylic acid (2-methoxyethyl)(2,2,2-trifluoroethyl)amide;
compound No. 26: ({2-Butyl-3-[4-(3-dibutylamino-propyl)benzoyl]indolizine-7-carbonyl}ethylamino)acetic acid ethyl ester;
compound No. 27: ({2-Butyl-3-[4-(3-dibutylamino-propyl)benzoyl]indolizine-7-carbonyl}ethylamino)acetic acid isopropyl ester;
compound No. 28: ({2-Butyl-3-[4-(3-dibutylamino-propyl)benzoyl]indolizine-7-carbonyl}isopropylamino)acetic acid methyl ester;
compound No. 29: ({3-[4-(3-dibutylaminopropyl)benzoyl]-2-ethylindolizine-7-carbonyl}isopropylamino)acetic acid methyl ester;
compound No. 30: ({3-[4-(3-Butylaminopropyl)benzoyl]-2-ethylindolizine-7-carbonyl}isopropylamino)acetic acid methyl ester;
compound No. 31: ({3-[4-(3-tert-Butylamino-propyl)benzoyl]-2-ethylindolizine-7-carbonyl}isopropylamino)acetic acid methyl ester;
compound No. 32: ({2-Ethyl-3-[4-(3-isopropylamino-propyl)benzoyl]indolizine-7-carbonyl}isopropylamino)acetic acid methyl ester;
compound No. 33: ({3-[4-(3-Cyclopentylamino-propyl)benzoyl]-2-ethylindolizine-7-carbonyl}ethylamino)acetic acid ethyl ester;
compound No. 34: ({3-[4-(3-Cyclopentylamino-propyl)benzoyl]-2-isopropylindolizine-7-carbonyl}isopropylamino)acetic acid methyl ester;
compound No. 35: ({3-[4-(3-Cyclohexylamino-propyl)benzoyl]-2-ethylindolizine-7-carbonyl}isopropylamino)acetic acid methyl ester;
compound No. 36: [(3-{4-[3(2,2-Dimethylpropyl-amino)propyl]benzoyl}-2-ethylindolizine-7-carbonyl)isopropylamino]acetic acid methyl ester;
compound No. 37: 3-[4-(3-Cyclopentylaminopropyl)benzoyl]-2-ethylindolizine-7-carboxylic acid (2-ethoxyethyl)ethylamide;
compound No. 38: 4-{3-[4-(3-tert-Butylamino-propyl)benzoyl]-2-ethylindolizine-7-carbonyl}piperazin-2-one;
compound No. 39: 2-Ethyl-3-{4-[3-(1-methyl-cyclopentylamino)propyl]benzoyl}indolizine-7-carboxylic acid ethyl(2-methyl-2H-tetrazol-5-ylmethyl)amide;
compound No. 40: 3-[4-(3-tert-Butylaminopropyl)benzoyl]-2-ethylindolizine-7-carboxylic acid ethyl(2-ethyl-2H-tetrazol-5-ylmethyl)amide;
compound No. 41: 3-[4-(3-tert-Butylaminopropyl)benzoyl]indolizine-7-carboxylic acid ethyl(2-methyl-2H-tetrazol-5-ylmethyl)amide;
compound No. 42: 3-[4-(3-tert-Butylaminopropyl)benzoyl]-2-cyclobutylindolizine-7-carboxylic acid ethyl(2-methyl-2H-tetrazol-5-ylmethyl)amide;
compound No. 43: 2-Ethyl-3-[4-(3-ethylamino-4,4-dimethylpentyl)benzoyl]indolizine-7-carboxylic acid ethyl(2-methyl-2H-tetrazol-5-ylmethyl)amide;
compound No. 44: 3-[4-(3-tert-Butylaminopropyl)benzoyl]-2-ethylindolizine-7-carboxylic acid ethyl(1-methyl-1H-pyrazol-3-ylmethyl)amide;
compound No. 45: 3-[4-(3-tert-Butylaminopropyl)benzoyl]-2-ethylindolizine-7-carboxylic acid ethyl(1-methyl-1H-pyrazol-4-ylmethyl)amide;
compound No. 46: 3-[4-(3-tert-Butylaminopropyl)benzoyl]-2-ethylindolizine-7-carboxylic acid ethyl(5-methylisoxazol-3-ylmethyl)amide;
compound No. 47: (R)-1-{3-[4-(3-tert-Butylamino-propyl)benzoyl]-2-ethylindolizine-7-carbonyl}pyrrolidine-3-carbonitrile;
compound No. 48: {3-[4-(3-tert-Butylaminopropyl)benzoyl]-2-ethylindolizin-7-yl}-((S)-3-hydroxypyrrolidin-1-yl)methanone;
compound No. 49: 3-[4-(3-tert-Butylaminopropyl)benzoyl]-2-ethylindolizine-7-carboxylic acid 2-methyl-2H-tetrazol-5-ylmethyl ester;
compound No. 50: (S)-1-[3-[4-(3-tert-Butylamino-propyl)benzoyl]-2-ethylindolizine-7-carbonyl]-2-methylpyrrolidine-2-carboxylic acid methyl ester;
compound No. 51: 3-[4-(3-tert-Butylaminopropyl)benzoyl]-2-ethylindolizine-7-carboxylic acid (R)-2-methoxy-1-methylethyl ester;
compound No. 52: 3-[4-(3-tert-Butylaminopropyl)benzoyl]-2-ethylindolizine-7-carboxylic acid (R)-5-oxopyrrolidin-3-yl ester;
compound No. 53: 2-{3-[4-(3-Cyclopentylamino-propyl)benzoyl]-2-ethylindolizine-7-carbonyl}[1,4]diazepam-5-one;
compound No. 54: [(3-{4-[3-(tert-Butylmethyl-amino)propyl]benzoyl}-2-ethylindolizine-7-carbonyl)iso-propylamino]acetic acid methyl ester;
compound No. 55: [(2-Ethyl-3-{4-[3-(ethylisopropyl-amino)propyl]benzoyl}indolizine-7-carbonyl)isopropylamino]acetic acid methyl ester;
compound No. 56: ({3-[4-(3-Dibutylamino-propyl)benzoyl]indolizine-7-carbonyl}isopropylamino)acetic acid methyl ester;
compound No. 57: ({3-[4-(3-Dibutylamino-propyl)benzoyl]indolizine-7-carbonyl}isopropylamino)acetic acid;
compound No. 58: ({2-Butyl-3-[4-(3-dibutyl-aminopropyl)benzoyl]indolizine-7-carbonyl}isopropylamino)acetic acid isopropyl ester;
compound No. 59: 2-Butyl-3-(4-piperidin-4-ylbenzoyl)indolizine-7-carboxylic acid diethylamide;
compound No. 60: ({2-Butyl-3-[4-(3-dibutylamino-propyl)benzoyl]indolizine-7-carbonyl}isopropylamino)acetic cid ethyl ester;
compound No. 61: ({3-[4-(3-Dipropylaminopropyl)benzoyl]-2-ethylindolizine-7-carbonyl}isopropylamino)acetic acid methyl ester;
compound No. 62: [(2-Butyl-3-{4-[3-(butylethyl-amino)propyl]benzoyl}indolizine-7-carbonyl)isopropylamino]acetic acid methyl ester;
compound No. 63: ({2-Butyl-3-[4-(3-dibutylamino-propyl)benzoyl]indolizine-7-carbonyl}isobutylamino)acetic acid methyl ester;
compound No. 64: ({2-Butyl-3-[4-(1-methylpiperidin-4-yl)benzoyl]indolizine-7-carbonyl}isopropylamino)acetic acid;
compound No. 65: {[2-Ethyl-3-(4-piperidin-4-yl-benzoyl)indolizine-7-carbonyl]isopropylamino}acetic acid methyl ester;
compound No. 66: 2-Butyl-3-(4-piperidin-4-yl-benzoyl)indolizine-7-carboxylic acid diethylamide;
compound No. 67: ({2-Butyl-3-[4-(piperidin-4-yloxy)benzoyl]indolizine-7-carbonyl}isopropylamino)acetic acid methyl ester;
compound No. 68: ({2-Butyl-3-[4-((S)-piperidin-3-yloxy)benzoyl]indolizine-7-carbonyl}isopropylamino)acetic acid methyl ester;
compound No. 69: (S)-2-({2-Butyl-3-[4-(3-dibutylaminopropyl)benzoyl]indolizine-7-carbonyl}ethyl-amino)propionic acid methyl ester;
compound No. 70: 2-Butyl-3-[4-(3-dibutyl-aminopropyl)benzoyl]indolizine-7-carboxylic acid benzylethylamide;
compound No. 71: 2-Butyl-3-[4-(3-butylamino-propyl)benzoyl]indolizine-7-carboxylic acid ethyl(2-methoxyethyl)amide;
compound No. 72: 2-Butyl-3-[4-(3-dibutylamino-propyl)benzoyl]indolizine-7-carboxylic acid (2-isopropoxyethyl)isopropylamide;
compound No. 73: [(2-Butyl-3-{4-[3-((3R,5S)-3,5-dimethylpiperidin-1-yl)propyl]benzoyl}indolizine-7-carbonyl)isopropylamino]acetic acid methyl ester;
compound No. 74: (Benzyl-{2-butyl-3-[4-(3-dibutyl-aminopropyl)benzoyl]indolizine-7-carbonyl}amino)acetic acid methyl ester;
compound No. 75: ([2-Butyl-3-[4-(3-diethylamino-propyl)benzoyl]indolizine-7-carbonyl]isopropylamino)acetic acid;
compound No. 76: ({3-[4-(3-tert-Butylamino-propyl)benzoyl]-2-ethylindolizine-7-carbonyl}iso-propylamino)acetic acid;
compound No. 77: 3-[4-(3-Cyclopentylaminopropyl)benzoyl]-2-ethylindolizine-7-carboxylic acid ethyl(2-methyl-2H-tetrazol-5-ylmethyl)amide;
compound No. 78: 3-[4-(3-tert-Butylaminopropyl)benzoyl]-2-methylindolizine-7-carboxylic acid ethyl(2-methyl-2H-tetrazol-5-ylmethyl)amide;
compound No. 79: [(2-Ethyl-3-{4-[3-(1-isopropylamino-cyclopentyl)propyl]benzoyl}indolizine-7-carbonyl)isopropylamino)acetic acid methyl ester;
compound No. 80: 2-Butyl-3-(4-piperidin-4-ylbenzoyl)indolizine-7-carboxylic acid ethyl(3-methyl-[1,2,4]oxadiazol-5-ylmethyl)amide;
compound No. 81: ({2-Butyl-3-[4-((R)-piperidin-3-yloxy)benzoyl]indolizine-7-carbonyl}isopropylamino)acetic acid methyl ester;
in the form of a base or of an addition salt with an acid.

3. The compound of formula (I) as claimed in claim 1, chosen from:
compound No. 3: (R)-1-{2-Butyl-3-[4-(3-dibutylamino-propyl)benzoyl]indolizine-7-carbonyl}pyrrolidine-2-carboxylic acid methyl ester;
compound No. 4: 2-Butyl-3-[4-(3-dibutylamino-propyl)benzoyl]indolizine-7-carboxylic acid ethyl(2-methoxyethyl)amide;
compound No. 5: ({2-Butyl-3-[4-(3-dibutylamino-propyl)benzoyl]indolizine-7-carbonyl}ethylamino)acetic acid methyl ester;
compound No. 8: ({3-[4-(3-Cyclopentylamino-propyl)benzoyl]2-ethylindolizine-7-carbonyl}isopropylamino)acetic acid methyl ester;
compound No. 9: 2-Butyl-3-[4-(3-dibutylamino-propyl)benzoyl]indolizine-7-carboxylic acid ethyl(2-methyl-2H-tetrazol-5-ylmethyl)amide;
compound No. 10: 2-Butyl-3-[4-(3-dibutylamino-propyl)benzoyl]indolizine-7-carboxylic acid ethyl(3-methyl[1,2,4]oxadiazol-5-ylmethyl)amide;
compound No. 13: 4-{2-Butyl-3-[4-(3-dibutylamino-propyl)benzoyl]indolizine-7-carbonyl}piperazin-2-one;
compound No. 16: [(2-Ethyl-3-{4-[3-(1-methylaminocyclopentyl)propyl]benzoyl}indolizine-7-carbonyl)isopropylamino]acetic acid methyl ester;
compound No. 17: 3-[4-(3-tert-Butylaminopropyl)benzoyl]-2-ethylindolizine-7-carboxylic acid ethyl(2-methyl-2H-tetrazol-5-ylmethyl)amide;
compound No. 18: 3-[4-(3-tert-Butylamino-3-methyl-butyl)benzoyl]-2-ethylindolizine-7-carboxylic acid ethyl(2-methyl-2H-tetrazol-5-ylmethyl)amide;
compound No. 22: 2-Butyl-3-[4-(3-dibutylamino-propyl)benzoyl]indolizine-7-carboxylic acid isopropyl(2-methoxyethyl)amide;
compound No. 23: 2-Butyl-3-[4-(3-dibutylamino-propyl)benzoyl]indolizine-7-carboxylic acid ethyl(2-isopropoxyethyl)amide;
compound No. 24: 2-Butyl-3-[4-(3-dibutylaminopropyl)benzoyl]indolizine-7-carboxylic acid (2-ethoxy-ethyl)isopropylamide;
compound No. 28: ({2-Butyl-3-[4-(3-dibutylamino-propyl)benzoyl]indolizine-7-carbonyl}isopropylamino)acetic acid methyl ester;
compound No. 29: ({3-[4-(3-Dibutylaminopropyl)benzoyl]-2-ethylindolizine-7-carbonyl}isopropylamino)acetic acid methyl ester;
compound No. 30: ({3-[4-(3-Butylaminopropyl)benzoyl]-2-ethylindolizine-7-carbonyl}isopropylamino)acetic acid methyl ester;
compound No. 31: ({3-[4-(3-tert-Butylamino-propyl)benzoyl]-2-ethylindolizine-7-carbonyl}iso-propylamino)acetic acid methyl ester;
compound No. 35: ({3-[4-(3-Cyclohexylamino-propyl)benzoyl]-2-ethylindolizine-7-carbonyl}isopropylamino)acetic acid methyl ester;
compound No. 40: 3-[4-(3-tert-Butylaminopropyl)benzoyl]-2-ethylindolizine-7-carboxylic acid ethyl(2-ethyl-2H-tetrazol-5-ylmethyl)amide;
compound No. 42: 3-[4-(3-tert-Butylaminopropyl)benzoyl-2-cyclobutylindolizine-7-carboxylic acid ethyl(2-methyl-2H-tetrazol-5-ylmethyl)amide;
compound No. 43: 2-Ethyl-3-[4-(3-ethylamino-4,4-dimethyl-pentyl)benzoyl]indolizine-7-carboxylic acid ethyl(2-methyl-2H-tetrazol-5-ylmethyl)amide;
compound No. 53: 1-{3-[4-(3-Cyclopentylaminopropyl)benzoyl]-2-ethylindolizine-7-carbonyl}[1,4]diazepam-5-one;
compound No. 55: [(2-Ethyl-3-{4-[3-(ethylisopropyl-amino)propyl]benzoyl}indolizine-7-carbonyl)isopropylamino]acetic acid methyl ester;
compound No. 58: ({3-[4-(3-Dibutylaminopropyl)benzoyl]-2-ethylindolizine-7-carbonyl}ethylamino)acetic acid isopropyl ester;
compound No. 62: [(2-Butyl-3-{4-[3-(butyl-ethylamino)propyl]benzoyl}indolizine-7-carbonyl)isopropylamino]acetic acid methyl ester;
compound No. 63: ({2-Butyl-3-[4-(3-dibutylamino-propyl)benzoyl]indolizine-7-carbonyl}isobutylamino)acetic acid methyl ester;
compound No. 64: ({2-Butyl-3-[4-(1-methylpiperidin-4-yl)benzoyl]indolizine-7-carbonyl}isopropylamino)acetic acid;
compound No. 65: {[2-Ethyl-3-(4-piperidin-4-ylbenzoyl)indolizine-7-carbonyl]isopropylamino}acetic acid methyl ester;
compound No. 69: (S)-2-({2-Butyl-3-[4-(3-dibutylamino-propyl)benzoyl]indolizine-7-carbonyl}ethylamino)propionic acid methyl ester;
compound No. 75: ({2-Butyl-3-[4-(3-diethylamino-propyl)benzoyl]indolizine-7-carbonyl}isopropylamino)acetic acid;
compound No. 77: 3-[4-(3-Cyclopentylaminopropyl)benzoyl]-2-ethylindolizine-7-carboxylic acid ethyl(2-methyl-2H-tetrazol-5-ylmethyl)amide;
compound No. 78: 3-[4-(3-tert-Butylaminopropyl)benzoyl]-2-methylindolizine-7-carboxylic acid ethyl(2-methyl-2H-tetrazol-5-ylmethyl)amide;
in the form of a base or of an addition salt with an acid.

4. A compound of formula (VI): 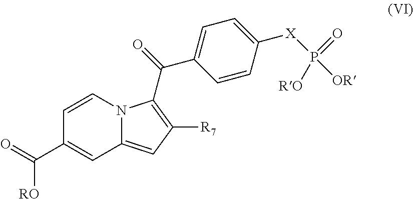
wherein:
R7 is as defined in claim 1;
R represents a (C₁-C₄) alkyl group;
R′ represents a (C₁-C₄) alkyl group;
P represents a phosphorus atom;
in the form of a base or of an addition salt with an acid.

5. A pharmaceutical composition comprising the compound of claim 1, or an addition salt of said compound with a pharmaceutically acceptable acid.

6. (canceled)

7. A method of preventing or treating atrial and ventricular arrhythmias: atrial tachyarrhythmia, atrial fibrillation, atrial flutter, atrial tachycardia, ventricular tachyarrhythmia, ventricular extrasystoles, ventricular tachycardia, ventricular flutter and fibrillation; angina pectoris, hypertension, cerebral circulatory insufficiency, heart failure, myocardium infarction which may or may not be complicated by heart failure, or preventing post-infarction mortality, or stroke in a patient in need thereof comprising administering to said patient a therapeutically effective amount of the pharmaceutical composition of claim 5.

8. A pharmaceutical composition, comprising a compound of claim 1, or a pharmaceutically acceptable salt, and also at least one pharmaceutically acceptable excipient.

9. A method of treating pathological syndromes of the cardiovascular system in a patient in need thereof comprising administering to said patient a therapeutically effective amount of the pharmaceutical composition of claim 8.

10. A method of preventing or treating atrial and ventricular arrhythmias: atrial tachyarrhythmia, atrial fibrillation, atrial flutter, atrial tachycardia, ventricular tachyarrhythmia, ventricular extrasystoles, ventricular tachycardia, ventricular flutter and fibrillation; angina pectoris, hypertension, cerebral circulatory insufficiency, heart failure, myocardium infarction which may or may not be complicated by heart failure, or preventing post-infarction mortality, or stroke in a patient in need thereof comprising administering to said patient a therapeutically effective amount of the pharmaceutical composition of claim 8.
